(19) United States
(12) Patent Application Publication
(10) Pub. No.: US 2026/0327193 A1
(43) Pub. Date: Oct. 22, 2026

(54) INHIBITORS OF PLASMA KALLIKREIN

(71) Applicant: Rezolute, Inc., Redwood City, CA (US)

(72) Inventors: Ian William David Yates, Bend, OR (US); Jeffrey Francis Breit, Bend, OR (US)

(21) Appl. No.: 19/099,427

(22) PCT Filed: Jul. 25, 2023
(86) PCT No.: PCT/US2023/028622

§ 371 Date: Jan. 28, 2025

Related U.S. Application Data
(60) Provisional application No. 63/393,427, filed on Jul. 29, 2022.
(60) Provisional application No. 63/460,204, filed on Apr. 18, 2023.

(51) Int. Cl. C07D 261/18; A61K 31/40; A61K 31/4155; A61K 31/4164; A61K 31/4192; A61K 31/42; A61K 31/425; C07D 207/34; C07D 231/14; C07D 233/90; C07D 249/04; C07D 275/03; C07D 403/10

(52) U.S. Cl. CPC C07D 261/18; A61K 31/40; A61K 31/4155; A61K 31/4164; A61K 31/4192; A61K 31/42; A61K 31/425; C07D 207/34; C07D 231/14; C07D 233/90; C07D 249/04; C07D 275/03; C07D 403/10

(57) ABSTRACT

The present disclosure provides compounds of Formula (IA) and their pharmaceutical compositions: The compounds and compositions are useful for inhibiting the activity of plasma kallikrein, and they are useful in therapy and in methods of treating diseases and conditions, such as ocular disorders.

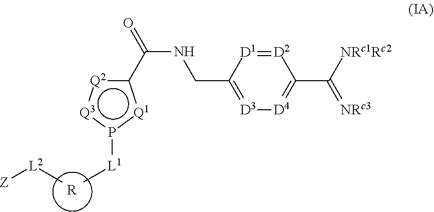

This application claims the benefit of priority to U.S. Provisional Patent Applications No. 63/393,427 filed on Jul. 29, 2022 and No. 63/460,204 filed on Apr. 18, 2023, which applications are incorporated in their entireties as if fully set forth herein.

### BACKGROUND

Plasma kallikrein is a serine protease that circulates in blood as prekallikrein, an inactive precursor, and participates in the surface-mediated defense system via activation of factor XII and high molecular weight kininogen (HK) involved signaling. Elements of the kallikrein-kinin system (KKS) are involved in activities such as surface-mediated defense reactions, regulation of blood flow, fibrin deposition, blood pressure, smooth muscle contractility, nociception, electrolyte transport, and mediator release. See Donald H. Miller, Harry S. Margolius, Chapter 19 The kallikrein-kinin-kininogen system, Editor(s): E. Edward Bittar, Neville Bittar, Principles of Medical Biology, Elsevier, Volume 8, 1997, Pages 363-384.

### SUMMARY

The present disclosure provides small molecule inhibitors of plasma kallikrein and methods of using the inhibitors to treat disease. Thus, in various embodiments, the present disclosure provides a compound of formula (IA) or a pharmaceutically acceptable salt thereof:

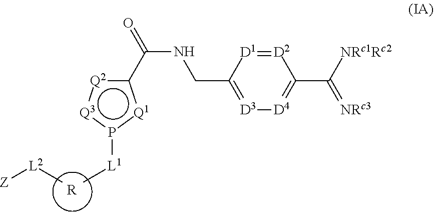

In Formula (IA), D¹ is N or CR¹, D² is N or CR², D³ is N or CR³, and D⁴ is N or CR⁴. Further, no more than three of D¹, D², D³, and D⁴ are simultaneously N.

Substituents R¹, R², R³, and R⁴ are independently selected from the group consisting of H, C₂-C₆-alkenyl, C₁-C₆-haloalkyl, halo, NR^{a}R^{b}, OR^{a}, —NR^{a} C(O)R^{b}, —C(O)R^{a}, —C(O)halo, —OC(O)R^{a}, —OC(O)OR^{a}, —C(O)OR^{a}, C₆-C₁₀-aryl, CN, —S(O)₀₋₂R^{a}, —S(O)₂OR^{a}, and NO₂.

Each R^{a} and R^{b} is independently selected from the group consisting of H, C₁-C₆-alkyl, and C₁-C₆-haloalkyl.

Substituents R^{c1}, R^{c2}, and R^{c3} are independently selected from group consisting of H, C₁-C₆-alkyl, and C₁-C₆-haloalkyl.

Q₁, Q₂, and Q₃ are independently selected from the group consisting of CR⁵, N, O and S.

R⁵ is selected from the group consisting of H, C₁-C₆-alkyl, OR^{a}, —(C₁-C₆-alkyl)OR^{a}, and C₃-C₁₀-cycloalkyl.

Ring member P is C or N.

L¹ is —SO₂— or —C₁-C₈-alkylene-.

The moiety

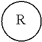

is a bivalent monocyclic or bicyclic moiety selected from the group consisting of C₃-C₁₀-cycloalkyl, C₆-C₁₀-aryl, 3- to 10-membered heterocycloalkyl (wherein 1-4 ring members are independently selected from N, O, and S), 5- to 10-membered heteroaryl (wherein 1-4 heteroaryl members are independently selected from N, O, and S), and fused combinations thereof.

L² is selected from the group consisting of a bond, —C₁-C₈-alkylene, —C₂-C₈-alkenylene, —C₂-C₈-alkynylene, and a bivalent moiety selected from the group consisting of C₃-C₁₀-cycloalkyl, C₃-C₁₀-cycloalkenyl, C₆-C₁₀-aryl, 5- to 10-membered heteroaryl (wherein 1-4 heteroaryl members are independently selected from N, O, and S), (C₁-C₈-alkyl) C₃-C₁₀-cycloalkyl, (C₁-C₈-alkyl) C₃-C₁₀-cycloalkenyl, (C₁-C₈-alkyl) C₆-C₁₀-aryl, and (C₁-C₈-alkyl)₅- to 10-membered heteroaryl (wherein 1-4 heteroaryl members are independently selected from N, O, and S). In L₂, any cycloalkyl, cycloalkenyl, aryl, and heteroaryl is monocyclic or bicyclic. L₂ is optionally substituted by 1 to 3 substituents selected from the group consisting of C₁-C₆-alkyl, C₁-C₆-haloalkyl, and CN.

Z is selected from the group consisting of —OR^{c}, —OC(O)R^{c}, —OC(O)NR^{c}R^{d}, —S(O)₀₋₂R^{c}, —CN, —C(O)R^{c}, —C(O)OR^{c}, —C(O)NR^{c}R⁴, —C(S)R^{c}, —NR^{c}R⁴, ═NR^{c}, —R^{c}C(O)NR^{c}R⁴, —R^{c}CO₂R⁴, —R^{c}—NO, —NO₂, —R^{c}—OR₄, —N═C═O, —N═C═S, and —NR^{c}—NR^{c}R^{d}, and

Each instance of R^{c} and R^{d} is independently selected from H, C₁-C₆-alkyl, C₁-C₆-haloalkyl, and C₆-C₁₀-aryl.

In another embodiment, the compound is one of Formula I:

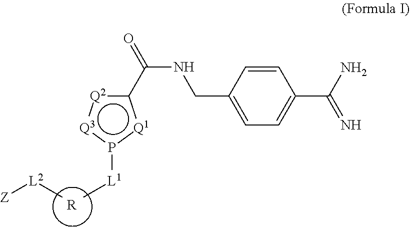

In Formula I, the moiety

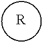

is i) a cyclic hydrocarbon, bicyclic hydrocarbon, or heterocycle containing up to 10 atoms consisting of C or N, or R is ii) selected from the group consisting of cyclopentyl, cyclohexyl, phenyl, naphthyl, pyridine, pyrimidine, indene, 2,3-dihydro-1H-indene or any saturated and unsaturated cyclic hydrocarbon or heterocycle thereof.

Z is selected from the group consisting of —OH, —OR′, —OC(O) H, —OC(O)R′, —OC(O)NH₂, —OC(O)NHR′, —OC(O)NH(R′)₂, —SH, —SR′, —S(O)R′, —S(O)₂R′, —CN, —C(O) H, —C(O)R′, —C(O)OH, —C(O)OR′, —C(O)NH₂, —C(O)NHR′, —C(O)NH(R′)₂, —C(S)R′, —NH₂, —NH₂R′, —R′2, ═NH, ═NR′, —NHC(O)NH₂, —NR′C(O)NH₂, —R′C(O)NHR′, —R′C(O)N(R′)₂, —NHCO₂H, —NHCO₂R′, —NR′CO₂R′, —NH—NO, —R′—NO, —NO₂, —NH—OH, —OH, —R′—OR′, —N═C—O, —N═C═S, —NH—NH₂, and —NH—NHR′, wherein each R′ is an independent alkyl or alkyl halide. In some embodiments, Z is not a halogen or hydrogen.

Q₁, Q₂ and Q₃ are selected from the group consisting of C, N, O or S.

P is selected from the group consisting of C or N.

L₁ is a linking group selected from the group consisting of a C₁-C₈ alkyl linker or SO₂.

L₂ is selected from i) a hydrocarbon that does not contain a double bond to O or S, ii) a hydrocarbon and does not contain a heteroatom such as O, N, or S; or iii) a linking group selected from the group consisting of a C₁-C₈ alkyl linker comprising a saturated hydrocarbon, an unsaturated hydrocarbon, a branched hydrocarbon, a cyclic hydrocarbon and combinations thereof; a cyclic hydrocarbon selected from the group consisting of cyclopentyl, cyclohexyl, phenyl, naphthyl, indene, 2,3-dihydro-1H-indene or any saturated or unsaturated cyclic hydrocarbon thereof. In some embodiments, L₂ is a hydrocarbon that contains a heteroatom such as O, N, or S

In some embodiments, the compositions comprise the compound of Formula I and pharmaceutically acceptable salts thereof.

### DETAILED DESCRIPTION

The present disclosure provides compounds, compositions, and methods for inhibiting plasma kallikrein (“PKal”). The compounds are useful, in exemplary embodiments, for the treatment of inflammatory and ocular disorders.

### Definitions

Unless defined otherwise, all technical and scientific terms used herein have the same meaning as commonly understood by one of ordinary skill in the art to which this disclosure pertains.

As used herein and in the appended claims, the singular forms “a,” “and,” and “the” include plural referents unless the context clearly dictates otherwise. Thus, for example, reference to “an agent” includes a plurality of such agents, and reference to “the cell” includes reference to one or more cells (or to a plurality of cells) and equivalents thereof known to those skilled in the art, and so forth.

When ranges are used herein for physical properties, such as molecular weight, or chemical properties, such as chemical formulae, all combinations and subcombinations of ranges and specific embodiments therein are intended to be included. The term “about” when referring to a number or a numerical range means that the number or numerical range referred to is an approximation within experimental variability (or within statistical experimental error), and thus the number or numerical range, in some instances, will vary between 1% and 15% of the stated number or numerical range.

In the present disclosure, the number of atoms of a particular element in a substituent group is generally given as a range, e.g., an alkyl group containing from 1 to 4 carbon atoms or C₁-4 alkyl. Reference to such a range is intended to include specific references to groups having each of the integer number of atoms within the specified range. For example, an alkyl group from 1 to 4 carbon atoms includes each of C₁, C₂, C₃, and C₄. A C₁-12 heteroalkyl, for example, includes from 1 to 12 carbon atoms in addition to one or more heteroatoms. Other numbers of atoms and other types of atoms may be indicated in a similar manner.

The term “comprising” (and related terms such as “comprise” or “comprises” or “having” or “including”) is not intended to exclude that in other certain embodiments, for example, an embodiment of any composition of matter, composition, method, or process, or the like, described herein, “consist of” or “consist essentially of” the described features.

“Alkyl” refers to straight or branched chain hydrocarbyl including from 1 to about 20 carbon atoms. For instance, an alkyl can have from 1 to 10 carbon atoms or 1 to 6 carbon atoms. Exemplary alkyl includes straight chain alkyl groups such as methyl, ethyl, propyl, butyl, pentyl, hexyl, heptyl, octyl, nonyl, decyl, undecyl, dodecyl, and the like, and also includes branched chain isomers of straight chain alkyl groups, for example without limitation, —CH(CH₃)₂, —CH(CH₃)(CH₂CH₃), —CH(CH₂CH₃)₂, —C(CH₃)₃, —C(CH₂ CH₃)₃, —CH₂CH(CH₃)₂, —CH₂CH(CH₃)(CH₂CH₃), —CH₂CH(CH₂CH₃)₂, —CH₂C(C H₃)₃, —CH₂C(CH₂CH₃)₃, —CH(CH₃)CH(CH₃)(CH₂CH₃), —CH₂CH₂CH(CH₃)₂, —CH 2CH₂CH(CH₃)(CH₂CH₃), —CH₂CH₂CH(CH₂CH₃)₂, —CH₂CH₂C(CH₃)₃, —CH₂CH₂C(CH₂CH₃)₃, —CH(CH₃) CH₂CH(CH₃)₂, —CH(CH₃)CH(CH₃)CH(CH₃)₂, and the like. Thus, alkyl groups include primary alkyl groups, secondary alkyl groups, and tertiary alkyl groups. An alkyl group can be unsubstituted or optionally substituted with one or more substituents as described herein, such as halogen(s), for example.

Each of the terms “halogen,” “halide,” and “halo” refers to —F or fluoro, —Cl or chloro, —Br or bromo, or —I or iodo.

The term “alkenyl” refers to straight or branched chain hydrocarbyl groups including from 2 to about 20 carbon atoms having 1-3, 1-2, or at least one carbon to carbon double bond. An alkenyl group can be unsubstituted or optionally substituted with one or more substituents as described herein.

“Alkyne or “alkynyl” refers to a straight or branched chain unsaturated hydrocarbon having the indicated number of carbon atoms and at least one triple bond. Examples of a (C₂-C₈)alkynyl group include, but are not limited to, acetylene, propyne, 1-butyne, 2-butyne, 1-pentyne, 2-pentyne, 1-hexyne, 2-hexyne, 3-hexyne, 1-heptyne, 2-heptyne, 3-heptyne, 1-octyne, 2-octyne, 3-octyne and 4-octyne. An alkynyl group can be unsubstituted or optionally substituted with one or more substituents as described herein.

The term “cycloalkyl” refers to a saturated monocyclic, bicyclic, tricyclic, or polycyclic, 3- to 14-membered ring system, such as a C₃-C₈-cycloalkyl. The cycloalkyl may be attached via any atom. Representative examples of cycloalkyl include, but are not limited to cyclopropyl, cyclobutyl, cyclopentyl, and cyclohexyl. A cycloalkyl group can be unsubstituted or optionally substituted with one or more substituents as described herein.

Embodiments of inhibitors of plasma kallikrein include compounds comprising a C₁-C₈ alkyl linker. In embodiments, a “C₁₋₈ alkyl” may be characterized by a branched or unbranched hydrocarbon group having from 1 to 8 carbon atoms. C₁-8 alkyls include, without limitation, methyl, ethyl, n-propyl, isopropyl, cyclopropyl, cyclopropylmethyl, n-butyl, iso-butyl, sec-butyl, tert-butyl, cyclobutyl, pentyl, and cyclopentyl.

“Aryl” (Ar) when used alone or as part of another term means a carbocyclic aromatic group whether or not fused having the number of carbon atoms designated or if no number is designated, up to 14 carbon atoms, such as a C₆-C₁₀-aryl or C₆-C₁₄-aryl. In embodiments, an Ar may be characterized by an aromatic group having a ring system comprised of carbon atoms with conjugated π electrons (e.g., phenyl). The term includes aryl groups having from 6 to 12 carbon atoms. Aryl groups may optionally include monocyclic, bicyclic, or tricyclic rings, in which each ring has five or six members. Examples of aryl groups include phenyl, naphthyl, biphenyl, phenanthrenyl, naphthacenyl, and the like (see e.g. Lang's Handbook of Chemistry (Dean, J. A., ed)₁₃^{th} ed. Table 7-2 [1985]). “Aryl” also contemplates an aryl ring that is part of a fused polycyclic system, such as aryl fused to cycloalkyl as defined herein. An exemplary aryl is phenyl. An aryl group can be unsubstituted or optionally substituted with one or more substituents as described herein.

The term “heteroatom” refers to N, O, and S. Compounds of the present disclosure that contain N or S atoms can be optionally oxidized to the corresponding N-oxide, sulfoxide, or sulfone compounds.

“Heteroaryl,” alone or in combination with any other moiety described herein, is a monocyclic aromatic ring structure containing 5 to 10, such as 5 or 6 ring atoms, or a bicyclic aromatic group having 8 to 10 atoms, containing one or more, such as 1-4, 1-3, or 1-2, heteroatoms independently selected from the group consisting of O, S, and N. Heteroaryl is also intended to include oxidized S or N, such as sulfinyl, sulfonyl and N-oxide of a tertiary ring nitrogen. A carbon or heteroatom is the point of attachment of the heteroaryl ring structure such that a stable compound is produced. Examples of heteroaryl groups include, but are not limited to, pyridinyl, pyridazinyl, pyrazinyl, quinaoxalyl, indolizinyl, benzo[b]thienyl, quinazolinyl, purinyl, indolyl, quinolinyl, pyrimidinyl, pyrrolyl, pyrazolyl, oxazolyl, thiazolyl, thienyl, isoxazolyl, oxathiadiazolyl, isothiazolyl, tetrazolyl, imidazolyl, triazolyl, furanyl, benzofuryl, and indolyl. A heteroaryl group can be unsubstituted or optionally substituted with one or more substituents as described herein.

“Heterocycloalkyl” is a saturated or partially unsaturated non-aromatic monocyclic, bicyclic, tricyclic or polycyclic ring system that has from 3 to 14, such as 3 to 6, atoms in which 1 to 3 carbon atoms in the ring are replaced by heteroatoms of O, S or N. The ring heteroatoms can also include oxidized S or N, such as sulfinyl, sulfonyl, and N-oxides of a tertiary ring nitrogen. A heterocycloalkyl can be fused to another ring system, such as with an aryl or heteroaryl of 5-6 ring members. The point of attachment of the heterocycloalkyl ring is at a carbon or heteroatom such that a stable ring is retained. Examples of heterocycloalkyl groups include without limitation morpholino, tetrahydrofuranyl, dihydropyridinyl, piperidinyl, pyrrolidinyl, piperazinyl, dihydrobenzofuryl, and dihydroindolyl. A heterocycloalkyl group can be unsubstituted or optionally substituted with one or more substituents as described herein.

The term “nitrile” or “cyano” can be used interchangeably and refers to a —CN group.

Compounds described herein can exist in various isomeric forms, including configurational, geometric, and conformational isomers, including, for example, cis- or trans-conformations. The compounds may also exist in one or more tautomeric forms, including both single tautomers and mixtures of tautomers. The term “isomer” is intended to encompass all isomeric forms of a compound of this disclosure, including tautomeric forms of the compound. The compounds of the present disclosure may also exist in open-chain or cyclized forms. In some cases, one or more of the cyclized forms may result from the loss of water. The specific composition of the open-chain and cyclized forms may be dependent on how the compound is isolated, stored or administered. For example, the compound may exist primarily in an open-chained form under acidic conditions but cyclize under neutral conditions. All forms are included in the disclosure.

Some compounds described herein can have asymmetric centers and therefore exist in different enantiomeric and diastereomeric forms. A compound as described herein can be in the form of an optical isomer or a diastereomer. Accordingly, the disclosure encompasses compounds and their uses as described herein in the form of their optical isomers, diastereoisomers and mixtures thereof, including a racemic mixture. Optical isomers of the compounds of the disclosure can be obtained by known techniques such as asymmetric synthesis, chiral chromatography, simulated moving bed technology or via chemical separation of stereoisomers through the employment of optically active resolving agents.

Unless otherwise indicated, the term “stereoisomer” means one stereoisomer of a compound that is substantially free of other stereoisomers of that compound. Thus, a stereomerically pure compound having one chiral center will be substantially free of the opposite enantiomer of the compound. A stereomerically pure compound having two chiral centers will be substantially free of other diastereomers of the compound. A typical stereomerically pure compound comprises greater than about 80% by weight of one stereoisomer of the compound and less than about 20% by weight of other stereoisomers of the compound, for example greater than about 90% by weight of one stereoisomer of the compound and less than about 10% by weight of the other stereoisomers of the compound, or greater than about 95% by weight of one stereoisomer of the compound and less than about 5% by weight of the other stereoisomers of the compound, or greater than about 97% by weight of one stereoisomer of the compound and less than about 3% by weight of the other stereoisomers of the compound, or greater than about 99% by weight of one stereoisomer of the compound and less than about 1% by weight of the other stereoisomers of the compound. The stereoisomer as described above can be viewed as composition comprising two stereoisomers that are present in their respective weight percentages described herein.

If there is a discrepancy between a depicted structure and a name given to that structure, then the depicted structure controls. Additionally, if the stereochemistry of a structure or a portion of a structure is not indicated with, for example, bold or dashed lines, the structure or portion of the structure is to be interpreted as encompassing all stereoisomers of it. In some cases, however, where more than one chiral center exists, the structures and names may be represented as single enantiomers to help describe the relative stereochemistry. Those skilled in the art of organic synthesis will know if the compounds are prepared as single enantiomers from the methods used to prepare them.

As used herein, and unless otherwise specified to the contrary, the term “compound” is inclusive in that it encompasses a compound or a pharmaceutically acceptable salt, stereoisomer, isotopologue, and/or tautomer thereof. Thus, for instance, a compound includes a pharmaceutically acceptable salt of a tautomer of the compound. Similarly, a compound of includes a pharmaceutically acceptable salt of an isotopologue of the compound.

In this disclosure, a “pharmaceutically acceptable salt” is a pharmaceutically acceptable, organic or inorganic acid or base salt of a compound described herein. Representative pharmaceutically acceptable salts include, e.g., alkali metal salts, alkali earth salts, ammonium salts, water-soluble and water-insoluble salts, such as the acetate, amsonate (4,4-diaminostilbene-2,2-disulfonate), benzenesulfonate, benzonate, bicarbonate, bisulfate, bitartrate, borate, bromide, butyrate, calcium, calcium edetate, camsylate, carbonate, chloride, citrate, clavulariate, dihydrochloride, edetate, edisylate, estolate, esylate, fiunarate, gluceptate, gluconate, glutamate, glycollylarsanilate, hexafluorophosphate, hexylresorcinate, hydrabamine, hydrobromide, hydrochloride, hydroxynaphthoate, iodide, isothionate, lactate, lactobionate, laurate, malate, maleate, mandelate, mesylate, methylbromide, methylnitrate, methylsulfate, mucate, napsylate, nitrate, N-methylglucamine ammonium salt, 3-hydroxy-2-naphthoate, oleate, oxalate, palmitate, pamoate (1,1-methene-bis-2-hydroxy-3-naphthoate, einbonate), pantothenate, phosphate/diphosphate, picrate, polygalacturonate, propionate, p-toluenesulfonate, salicylate, stearate, subacetate, succinate, sulfate, sulfosaliculate, suramate, tannate, tartrate, teoclate, tosylate, triethiodide, and valerate salts. A pharmaceutically acceptable salt can have more than one charged atom in its structure. In this instance the pharmaceutically acceptable salt can have multiple counterions. Thus, a pharmaceutically acceptable salt can have one or more charged atoms and/or one or more counterions.

The terms “treat”, “treating” and “treatment” refer to the amelioration or eradication of a disease or symptoms associated with a disease. In various embodiments, the terms refer to minimizing or slowing the spread, progression, or worsening of the disease resulting from the administration of one or more prophylactic or therapeutic compounds described herein to a patient with such a disease.

The terms “prevent,” “preventing,” and “prevention” refer to the prevention of the onset, recurrence, or spread of the disease in a patient resulting from the administration of a compound described herein.

The term “effective amount” refers to an amount of a compound as described herein or other active ingredient sufficient to provide a therapeutic or prophylactic benefit in the treatment or prevention of a disease or to delay or minimize symptoms associated with a disease. Further, a therapeutically effective amount with respect to a compound as described herein means that amount of therapeutic agent alone, or in combination with other therapies, that provides a therapeutic benefit in the treatment or prevention of a disease. Used in connection with a compound as described herein, the term can encompass an amount that improves overall therapy, reduces or avoids symptoms or causes of disease, or enhances the therapeutic efficacy of or is synergistic with another therapeutic agent.

A “patient” or subject” includes an animal, such as a human, cow, horse, sheep, lamb, pig, chicken, turkey, quail, cat, dog, mouse, rat, rabbit or guinea pig. In accordance with some embodiments, the animal is a mammal such as a non-primate and a primate (e.g., monkey and human). In one embodiment, a patient is a human, such as a human infant, child, adolescent or adult. In the present disclosure, the terms “patient” and “subject” are used interchangeably.

### Inhibitors of Plasma Kallikrein

### Compound of Formula IA

In some embodiments, the inhibitor is a compound of formula (IA) or a pharmaceutically acceptable salt thereof:

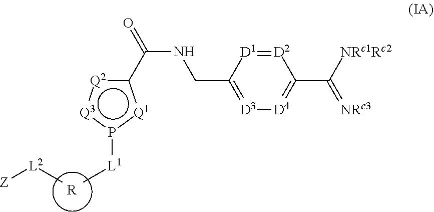

In Formula (IA), D¹ is N or CR¹, D² is N or CR², D³ is N or CR³, and D⁴ is N or CR⁴. Further, no more than three of D¹, D², D³, and D⁴ are simultaneously N.

Substituents R¹, R², R³, and R⁴ are independently selected from the group consisting of H, C₂-C₆-alkenyl, C₁-C₆-haloalkyl, halo, NR^{a}R^{b}, OR^{a}, —NR^{a} C(O)R^{b}, —C(O)R^{a}, —C(O)halo, —OC(O)R^{a}, —OC(O)OR^{a}, —C(O)OR^{a}, C₆-C₁₀-aryl, CN, —S(O)₀₋₂R^{a}, —S(O)₂OR^{a}, and NO₂.

Each R^{a} and R^{b} is independently selected from the group consisting of H, C₁-C₆-alkyl, and C₁-C₆-haloalkyl.

Substituents R^{c1}, R^{c2}, and R^{c3} are independently selected from group consisting of H, C₁-C₆-alkyl, and C₁-C₆-haloalkyl.

Q₁, Q₂, and Q₃ are independently selected from the group consisting of CR⁵, N, O and S.

R⁵ is selected from the group consisting of H, C₁-C₆-alkyl, OR^{a}, —(C₁-C₆-alkyl) OR^{a}, and C₃-C₁₀-cycloalkyl.

Ring member P is C or N.

L¹ is —SO₂— or —C₁-C₈-alkylene-.

The moiety

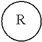

is a bivalent monocyclic or bicyclic moiety selected from the group consisting of C₃-C₁₀-cycloalkyl, C₆-C₁₀-aryl, 3- to 10-membered heterocycloalkyl (wherein 1-4 ring members are independently selected from N, O, and S), 5- to 10-membered heteroaryl (wherein 1-4 heteroaryl members are independently selected from N, O, and S), and fused combinations thereof.

L² is selected from the group consisting of a bond, —C₁-C₈-alkylene, —C₂-C₈-alkenylene, —C₂-C₈-alkynylene, and a bivalent moiety selected from the group consisting of C₃-C₁₀-cycloalkyl, C₃-C₁₀-cycloalkenyl, C₆-C₁₀-aryl, 5- to 10-membered heteroaryl (wherein 1-4 heteroaryl members are independently selected from N, O, and S), (C₁-C₈-alkyl) C₃-C₁₀-cycloalkyl, (C₁-C₈-alkyl) C₃-C₁₀-cycloalkenyl, (C₁-C₈-alkyl) C₆-C₁₀-aryl, and (C₁-C₈-alkyl)₅- to 10-membered heteroaryl (wherein 1-4 heteroaryl members are independently selected from N, O, and S). In L₂, any cycloalkyl, cycloalkenyl, aryl, and heteroaryl is monocyclic or bicyclic. L₂ is optionally substituted by 1 to 3 substituents selected from the group consisting of C₁-C₆-alkyl, C₁-C₆-haloalkyl, and CN.

Z is selected from the group consisting of —OR^{c}, —OC(O)R^{c}, —OC(O)NR^{c}R⁴, —S(O)₀₋₂R^{c}, —CN, —C(O)R^{e}, —C(O)OR^{c}, —C(O)NR^{c}R⁴, —C(S)R^{c}, —NR^{c}R⁴, ═NR^{c}, —R^{c}C(O)NR^{c}R⁴, —R^{c}CO₂R^{d}, —R^{c}—NO, —NO₂, —R^{c}—OR₄, —N═C═O, —N═C═S, and —NR^{c}—NR^{c}R^{d}, and

Each instance of R^{c} and R^{d} is independently selected from H, C₁-C₆-alkyl, C₁-C₆-haloalkyl, and C₆-C₁₀-aryl.

In some embodiments, three of D¹, D², D³, and D⁴ are N. In other embodiments, two of D¹, D², D³, and D⁴ are N. In still other embodiments, one of D¹, D², D³, and D⁴ is N. In still additional embodiments, no more than one of D¹, D², D³, and D⁴ is N. In illustrative embodiments, the ring containing D¹, D², D³, and D⁴ is selected from the group consisting of:

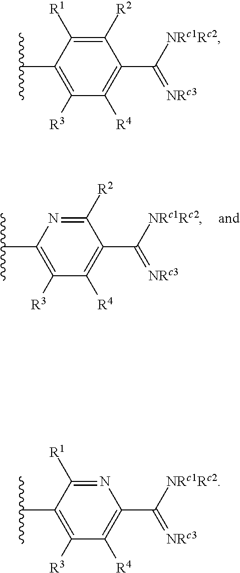

In a particular embodiment, the ring containing D¹, D², D³, and D⁴ is:

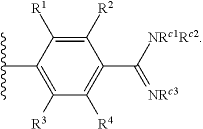

In various embodiments, R¹, R², R³, and R⁴ are independently selected from the group consisting of H, OR^{a}, halo, and CN. In some embodiments, R¹, R², R³, and R⁴ are independently selected from the group consisting of H and halo. For example, in an embodiment, at least one of R¹, R², R³, and R⁴ is halo. In another embodiment, each of R¹, R², R³, and R⁴ is H.

Some compounds of Formula (IA), in accordance with various embodiments, are those in which each of R^{c1}, R^{c2}, and R^{c3} is H. Thus, for example, some embodiments provide for compounds in which the ring containing D¹, D², D³, and D⁴ is:

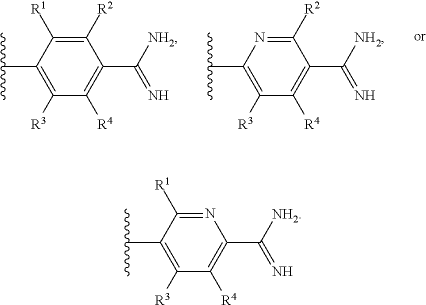

In various embodiments, Q₁ is C; each of Q₂ and Q₃ is independently selected from the group consisting of CR⁵, N, O and S; and P is selected from the group consisting of C and N. In one embodiment, P is N. In additional embodiments, Q₂ is CR⁵ and Q₃ is N, or Q₂ is N and Q₃ is CR⁵. Illustrative embodiments include compounds in which the ring containing P, Q₁, Q′, and Q₃ is:

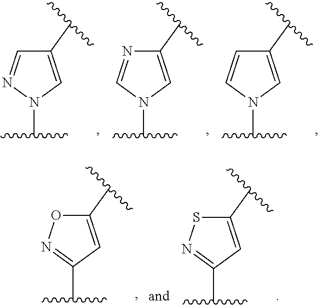 [SMILES: *c1cnn(*)c1 ; *c1cn(*)cn1 ; *c1ccn(*)c1 ; *c1cc(*)on1 ; *c1cc(*)sn1]

In additional embodiments, L¹ is-C₁-C₈-alkylene-, such as —C₁-C₃-alkylene-. In an illustrative embodiment, L¹ is methylene.

In an exemplary embodiment,

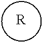

is a bivalent monocyclic C₆-C₁₀-aryl. In an exemplary embodiment,

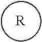

is

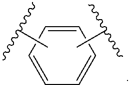

The present disclosure provides, in additional embodiments, compounds of Formula (IA) wherein L₂ is selected from −C₁-C₈-alkylene, C₆-C₁₀-aryl, and —(C₁-C₈-alkyl) C₆-C₁₀-aryl, any of which is optionally substituted as described herein. In some embodiments, L₂ is-C₁-C₈-alkylene, such as —C₁-C₃-alkylene. As generally described herein, the alkyl (or alkylene) moieties can be straight or branched. In other embodiments L₂ is C₆-C₁₀-aryl, including phenyl.

The moiety Z, in some embodiments, is selected from the group consisting of —OR^{c}, CN, —C(O)OR^{c}, and —C(O)NR^{c}R^{d}. In various embodiments, each R^{c} and R^{d} is independently H or C₁-C₆-alkyl. Thus, for example, Z can be selected from the group consisting of OH, OCH₃, —COOH, —C(O)NH₂, and —C(O)NHCH₃. In another embodiment, Z is CN.

### Compound of Formula I

In some embodiments, the present disclosure provides an inhibitor of PKal of Formula I.

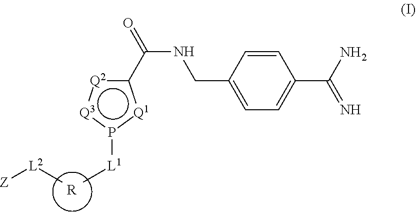

wherein R is i) a cyclic hydrocarbon, bicyclic hydrocarbon, or heterocycle containing up to 10 atoms consisting of C or N, or R is ii) selected from the group consisting of cyclopentyl, cyclohexyl, phenyl, naphthyl, pyridine, pyrimidine, indene, 2,3-dihydro-1H-indene or any saturated and unsaturated cyclic hydrocarbon or heterocycle thereof.

In some embodiments, Z is selected from the group consisting of —OH, —OR′, —OC(O) H, —OC(O)R′, —OC(O)NH₂, —OC(O)NHR′, —OC(O)NH(R′)₂, —SH, —SR′, —S(O)R′, —S(O)₂R′, —CN, —C(O) H, —C(O)R′, —C(O)OH, —C(O)OR′, —C(O)NH₂, —C(O)NHR′, —C(O)NH(R′)₂, —C(S)R′, —NH₂, —NH₂R′, —R′2, ═NH, ═NR′, —NHC(O)NH₂, —R′C(O)NH₂, —R′C(O)NHR′, —R′C(O)N(R′)₂, —NHCO₂H, —NHCO₂R′, —R′CO₂R′, —NH—NO, —R′—NO, —NO₂, —NH—OH, —OH, —NR′—OR′, —N═C═O, —N═C═S, —NH—NH₂, and —NH—NHR′, wherein each R′ is an independent alkyl or alkyl halide. In some embodiments, Z is not a halogen or hydrogen.

In some embodiments, Q₁, Q₂ and Q₃ are selected from the group consisting of C, N, O or S.

In some embodiments, P is selected from the group consisting of C or N.

In some embodiments, L is a linking group selected from the group consisting of a C₁-C₈ alkyl linker or SO₂.

In some embodiments, L₂ is selected from i) a hydrocarbon that does not contain a double bond to O or S, ii) a hydrocarbon and does not contain a heteroatom such as O, N, or S; or iii) a linking group selected from the group consisting of a C₁-C₈ alkyl linker comprising a saturated hydrocarbon, an unsaturated hydrocarbon, a branched hydrocarbon, a cyclic hydrocarbon and combinations thereof; a cyclic hydrocarbon selected from the group consisting of cyclopentyl, cyclohexyl, phenyl, naphthyl, indene, 2,3-dihydro-1H-indene or any saturated or unsaturated cyclic hydrocarbon thereof. In some embodiments, L₂ is a hydrocarbon that contains a heteroatom such as O, N, or S.

In some embodiments, the compositions comprise the compound of Formula I and pharmaceutically acceptable salts thereof.

In some embodiments, the compound comprises a subgenus wherein Q₁, Q₂ and Q₃ are selected from the group consisting of C and N, P is N, L₁ is CH₂, R is para-substituted benzene, L₂ is a methylene or ethylene alkyl linker in the para-position of the R group, and Z is selected from the group consisting of CN and OH.

Additional embodiments include those listed below:
Q₁ is C, Q₂ is C, Q₃ is N, L₂ is a methylene linker, and Z is CN;
Q₁ is C, Q₂ is C, Q₃ is N, L₂ is a methylene linker and Z is OH.
Q₁ is C, Q₂ is C, Q₃ is N, L₂ is an ethylene linker and Z is CN;
Q₁ is C, Q₂ is N, Q₃ is C, L₂ is an ethylene linker and Z is CN;
Q₁, Q₂, and Q₃ are selected from the group consisting of C or N, P is N, L¹ is CH₂, R is benzene, L₂ is selected from the group consisting of a branched ethyl linker and an isopropyl linker in the para-position of the R group and Z is a nitrile group (CN);
Q₁ is C, Q₂ is C, Q₃ is N, and L₂ is a branched ethyl linker;
Q₁ is C, Q₂ is N, Q₃ is C, and L₂ is a branched ethyl linker;
Q₁ is C, Q₂ is C, Q₃ is N, and L₂ is an isopropyl linker;
Q₁ is C, Q₂ is N, Q₃ is C, and L₂ is an isopropyl linker;
Q₁ is C, Q₂ is selected from the group consisting of N, O or S, Q₃ is selected from the group consisting of C or N, L₁ is a methylene or ethylene linker, R is benzene, L₂ is a methylene linker in the para-position of the R group, and Z is CN;
Q₂ is N, Q₃ is C, P is N, and L₁ is a methylene linker;
Q₂ is O, Q₃ is N, P is C, and L₁ is a methylene linker;
Q₂ is S, Q₃ is N, P is C, and L₁ is a methylene linker;
Q₂ is C, Q₃ is N, P is N, and L₁ is an ethylene linker;
Q₁ is C, Q₂ is C, Q₃ is N, P is N, L₁ is CH₂, R is benzene, L₂ is a phenyl linker in the para-position of the R group, and Z is CN;
Q₁ is C, Q₂ is C, Q₃ is N, P is N, L₁ is CH₂, R is benzene, L₂ is a benzyl linker in the para-position of the R group, and Z is CN in the para position;
Q₁ is C, Q₂ is C, Q₃ is N, P is N, L₁ is CH₂, R is benzene, L₂ is a methylene or ethylene linker in the para-position of the R group, and Z is selected from the group consisting of a methoxy group, carboxylic acid, an amide, and an amide substituted with a C₁ alkyl (methyl) group bonded to the N;
Q₁ is C, Q₂ is C, Q₃ is N, P is N, L₁ is CH₂, R is benzene, L₂ is an ethylene linker in the para-position of the R group, and Z is a methoxy group;
Q₁ is C, Q₂ is C, Q₃ is N, P is N, L₁ is CH₂, R is benzene, L₂ is a methylene linker in the para-position of the R group, and Z is carboxylic acid;
Q₁ is C, Q₂ is C, Q₃ is N, P is N, L₁ is CH₂, R is benzene, L₂ is a methylene linker in the para-position of the R group, and Z is an amide;
Q₁ is C, Q₂ is C, Q₃ is N, P is N, L₁ is CH₂, R is benzene, L₂ is a methylene linker in the para-position of the R group, and Z is an amide substituted with a C₁ alkyl (methyl) group bonded to the N;
Q₁ is C, Q₂ is C, Q₃ is N, P is N, L₁ is CH₂, R is benzene, L₂ is cyclopentadiene or comprises a methylene linker at position 1 of cyclopentadiene bonded to the para-position of the R group, and Z is CN bonded to position 3 of cyclopentadiene;
Q₁ is C, Q₂ is C, Q₃ is N, P is N, L₁ is CH₂, R is benzene, L₂ is cyclopentadiene bonded to the para-position of the R group, and Z is CN bonded to position 3 of cyclopentadiene; and.
Q₁ is C, Q₂ is C, Q₃ is N, P is N, L₁ is CH₂, R is benzene, L₂ is cyclopentadiene with a methylene linker at position 1 bonded to the para-position of the R group, and Z is CN bonded to position 3 of cyclopentadiene.

Additional embodiments include those listed below:
Q₁ is C, Q₂ is N, Q₃ is C, P is N, L₁ is CH₂, B is 1H-indene, and Z is CN; and
Q₁ is C, Q₂ is C, Q₃ is N, P is N, L₁ is CH₂, B is 2,3-dihydro-1H-indene, and Z is CN.

### Compound of Formula IB

In additional embodiments, optionally in combination with any other embodiment described herein, the present disclosure provides compound of formula (IB) or a pharmaceutically acceptable salt thereof:

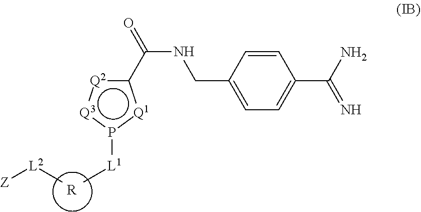

Q₁, Q₂, and Q₃ are independently selected from the group consisting of CR⁵, N, O and S.

R⁵ is selected from the group consisting of H, C₁-C₆-alkyl, OR^{a}, —(C₁-C₆-alkyl) OR^{a}, and C₃-C₁₀-cycloalkyl, wherein each R^{a} is independently selected from the group consisting of H, C₁-C₆-alkyl, and C₁-C₆-haloalkyl.

P is C or N.

L¹ is —SO₂— or —C₁-C₈-alkylene-.

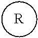

is a bivalent monocyclic or bicyclic moiety selected from the group consisting of C₃-C₁₀-cycloalkyl, C₆-C₁₀-aryl, 3- to 10-membered heterocycloalkyl (wherein 1-4 ring members are independently selected from N, O, and S), 5- to 10-membered heteroaryl (wherein 1-4 heteroaryl members are independently selected from N, O, and S), and fused combinations thereof.

L² is selected from the group consisting of a bond, —C₁-C₈-alkylene, —C₂-C₈-alkenylene, —C₂-C₈-alkynylene, and a bivalent moiety selected from the group consisting of C₃-C₁₀-cycloalkyl, C₃-C₁₀-cycloalkenyl, C₆-C₁₀-aryl, 5- to 10-membered heteroaryl (wherein 1-4 heteroaryl members are independently selected from N, O, and S), (C₁-C₈-alkyl) C₃-C₁₀-cycloalkyl, (C₁-C₈-alkyl) C₃-C₁₀-cycloalkenyl, (C₁-C₈-alkyl) C₆-C₁₀-aryl, and (C₁-C₈-alkyl)₅- to 10-membered heteroaryl (wherein 1-4 heteroaryl members are independently selected from N, O, and S), wherein cycloalkyl, cycloalkenyl, aryl, and heteroaryl are monocyclic or bicyclic.

L² is optionally substituted by 1 to 3 substituents selected from the group consisting of C₁-C₆-alkyl, C₁-C₆-haloalkyl, and CN.

Z is selected from the group consisting of —OR^{c}, —OC(O)R^{c}, —OC(O)NR^{c}R^{d}, —S(O)₀₋₂R^{c}, —CN, —C(O)R^{e}, —C(O)OR^{c}, —C(O)NR^{c}R^{d}, —C(S)R^{e}, —NR^{c}R⁴, ═NR^{c}, —R^{c}C(O)NR^{c}R^{d}, —R^{c}CO₂R⁴, —R^{c}—NO, —NO₂, —R^{c}—OR^{d}, —N═C═O, —N═C—S, and —NR^{c}—NR^{c}R^{d}.

Each instance of R^{c} and R^{d} is independently selected from H, C₁-C₆-alkyl, C₁-C₆-haloalkyl, and C₆-C₁₀-aryl.

### Compound of Formula IB-1

In additional embodiments, optionally in combination with any other embodiment described herein, the present disclosure provides compound of formula (IB-1) or a pharmaceutically acceptable salt thereof:

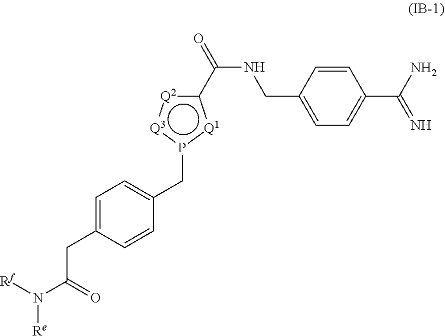

Q₁, Q₂, and Q₃ are independently selected from the group consisting of CR⁵, N, O and S.

R⁵ is selected from the group consisting of H, C₁-C₆-alkyl, OR^{a}, —(C₁-C₆-alkyl) OR^{a}, and C₃-C₁₀-cycloalkyl, wherein each R^{a} is independently selected from the group consisting of H, C₁-C₆-alkyl, and C₁-C₆-haloalkyl.

P is C or N.

R^{e} and R^{f} are each independently selected from the group consisting of H, C₁-C₆-alkyl, and C₁-C₆-haloalkyl.

### Compound of Formula IC

In additional embodiments, optionally in combination with any other embodiment described herein, the present disclosure provides compound of formula (IC) or a pharmaceutically acceptable salt thereof:

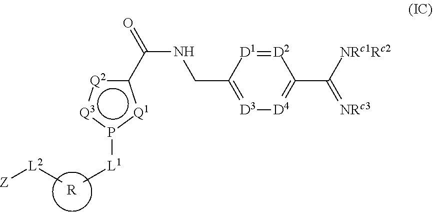

D¹ is N or CR¹, D² is N or CR², D³ is N or CR³, and D⁴ is N or CR⁴, wherein no more than three of D¹, D², D³, and D⁴ are simultaneously N.

R¹, R², R³, and R⁴ are independently selected from the group consisting of H, C₂-C₆-alkenyl, C₁-C₆-haloalkyl, halo, NR^{a}R^{b}, OR^{a}, —NR^{a} C(O)R^{b}, —C(O)R^{a}, —C(O)halo, —OC(O)R^{a}, —OC(O)OR^{a}, —C(O)OR^{a}, C₆-C₁₀-aryl, CN, —S(O)₀₋₂R^{a}, —S(O)₂OR^{a}, and NO₂.

Each R^{a} and R^{b} is independently selected from the group consisting of H, C₁-C₆-alkyl, and C₁-C₆-haloalkyl.

R^{c1}, R^{c2}, and R^{c3} are independently selected from group consisting of H, C₁-C₆-alkyl, and C₁-C₆-haloalkyl.

Q₁, Q₂, and Q₃ are independently selected from the group consisting of CR⁵, N, O and S.

R⁵ is selected from the group consisting of H, C₁-C₆-alkyl, OR^{a}, —(C₁-C₆-alkyl) OR^{a}, and C₃-C₁₀-cycloalkyl.

P is C or N.

L¹ is —SO— or —C₁-C₈-alkylene-.

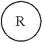

is a bivalent monocyclic or bicyclic moiety selected from the group consisting of C₃-C₁₀-cycloalkyl, C₆-C₁₀-aryl, 3- to 10-membered heterocycloalkyl (wherein 1-4 ring members are independently selected from N, O, and S), 5- to 10-membered heteroaryl (wherein 1-4 heteroaryl members are independently selected from N, O, and S), and fused combinations thereof.

L² is selected from the group consisting of a bond, —C₁-C₈-alkylene, —C₂-C₈-alkenylene, —C₂-C₈-alkynylene, and a bivalent moiety selected from the group consisting of C₃-C₁₀-cycloalkyl, C₃-C₁₀-cycloalkenyl, C₆-C₁₀-aryl, 5- to 10-membered heteroaryl (wherein 1-4 heteroaryl members are independently selected from N, O, and S), (C₁-C₈-alkyl) C₃-C₁₀-cycloalkyl, (C₁-C₈-alkyl) C₃-C₁₀-cycloalkenyl, (C₁-C₈-alkyl) C₆-C₁₀-aryl, and (C₁-C₈-alkyl)₅- to 10-membered heteroaryl (wherein 1-4 heteroaryl members are independently selected from N, O, and S), wherein cycloalkyl, cycloalkenyl, aryl, and heteroaryl are monocyclic or bicyclic.

L² is optionally substituted by 1 to 3 substituents selected from the group consisting of C₁-C₆-alkyl, C₁-C₆-haloalkyl, and CN.

Z is selected from the group consisting of —OR^{c}, —OC(O)R^{c}, —OC(O)NR^{c}R^{d}, —S(O)₀₋₂R^{c}, —CN, —C(O)R^{c}, —C(O)OR^{c}, —C(O)NR^{c}R⁴, —C(S)R^{c}, —NR^{c}R^{d}, ═NR^{c}, —R^{c}C(O)NR^{c}R^{d}, —R^{c}CO₂R^{d}, —R^{c}—NO, —NO₂, —R^{c}—OR₄, —N═C—O, —N═C═S, and —NR^{c}—NR^{c}R^{d}.

Each instance of R^{c} and R^{d} is independently selected from H, C₁-C₆-alkyl, C₁-C₆-haloalkyl, and C₆-C₁₀-aryl.

At least one of D¹, D², D³, or D⁴ is other than CH or at least one of R^{c1}, R^{c2}, or R^{c3} is other than H.

Illustrative embodiments of the present disclosure reside in specific compounds presented in the tables and the examples as described herein.

### Pharmaceutical Composition

The disclosure also provides a pharmaceutical composition comprising a therapeutically effective amount of one or more compounds as described herein, or a pharmaceutically acceptable salt, stereoisomer, and/or tautomer thereof in admixture with a pharmaceutically acceptable carrier. In some embodiments, the composition further contains, in accordance with accepted practices of pharmaceutical compounding, one or more additional therapeutic agents, pharmaceutically acceptable excipients, diluents, adjuvants, stabilizers, emulsifiers, preservatives, colorants, buffers, flavor imparting agents. The pharmaceutical composition can be administered by any suitable means that results in a concentration of the compound in a subject that is effective at treating a disease or condition suitable for treatment with the compounds of the disclosure.

In some embodiments, the compound is present in an amount of 1-95% by weight of the total weight of the composition. The “therapeutically effective amount” of a compound or a pharmaceutically acceptable salt, stereoisomer, and/or tautomer thereof that is administered is governed by such considerations, and is the minimum amount necessary to elicit inhibition of PKal. Such amount may be below the amount that is toxic to normal cells, or the subject as a whole. Generally, the initial therapeutically effective amount of a compound (or a pharmaceutically acceptable salt, stereoisomer, or tautomer thereof) of the present disclosure that is administered is in the range of about 0.001 to about 200 mg/kg or about 0.1 to about 20 mg/kg of patient body weight per day, with the typical initial range being about 0.3 to about 15 mg/kg/day. Oral unit dosage forms, such as tablets and capsules, may contain from about 0.1 mg to about 1000 mg of a compound (or a pharmaceutically acceptable salt, stereoisomer, or tautomer thereof) of the present disclosure. In another embodiment, such dosage forms contain from about 50 mg to about 500 mg of a compound (or a pharmaceutically acceptable salt, stereoisomer, or tautomer thereof) of the present disclosure. In yet another embodiment, such dosage forms contain from about 25 mg to about 200 mg of a compound (or a pharmaceutically acceptable salt, stereoisomer, or tautomer thereof) of the present disclosure. In still another embodiment, such dosage forms contain from about 10 mg to about 100 mg of a compound (or a pharmaceutically acceptable salt, stereoisomer, or tautomer thereof) of the present disclosure. In a further embodiment, such dosage forms contain from about 5 mg to about 50 mg of a compound (or a pharmaceutically acceptable salt, stereoisomer, or tautomer thereof) of the present disclosure. In other embodiments, the compound is administered into a dosage form for administration into or around the eye, as described herein, for which the therapeutically effective amount of the compound can range from about 0.0005 mg/kg to about 0.005 mg/kg, about 0.0007 mg/kg to about 0.004 mg/kg, or about 0.001 mg/kg to about 0.003 mg/kg of patient body weight. In any of the foregoing embodiments the dosage form can be administered once a day or twice per day.

While the attending physician ultimately will decide the appropriate amount and dosage regimen, in addition embodiments, the therapeutically effective amount of a compound described herein can be, for example, in the range of 0.0035 μg to 20 μg/kg body weight/day or 0.010 μg to 140 μg/kg body weight/week. In some embodiments, a therapeutically effective amount is in the range of 0.025 μg to 10 μg/kg, for example, at least 0.025, 0.035, 0.05, 0.075, 0.1, 0.25, 0.5, 1.0, 1.5, 2.0, 2.5, 3.0, 3.5, 4.0, 5.0, 6.0, 7.0, 8.0, or 9.0 μg/kg body weight administered daily, every other day, or twice a week. In some embodiments, a therapeutically effective amount may be in the range of 0.05 μg to 20 μg/kg, for example, at least 0.05, 0.7, 0.15, 0.2, 1.0, 2.0, 3.0, 4.0, 5.0, 6.0, 7.0, 8.0, 10.0, 12.0, 14.0, 16.0, or 18.0 μg/kg body weight administered weekly, every other week, or once a month. In some embodiments, a therapeutically effective amount of a compound may be, for example, in the range of 100 μg/m² to 100,000 μg/m² (of subject body surface area) administered daily, every other day, once weekly, or every other week. In some embodiments, the therapeutically effective amount is in the range of 1000 g/m² to 20,000 μg/m², for example, at least 1000, 1500, 4000, or 14,000 μg/m² of the compound administered daily, every other day, twice weekly, weekly, or every other week.

In some embodiments, a compounds of the disclosure is administered at a dose of about 0.01 mg to 1000 mg (e.g., 0.01, 0.05, 0.1, 0.2, 0.3, 0.4, 0.5, 0.6, 0.7, 0.8, 0.9, 1.0, 2.0, 3.0, 4.0, 5.0, 6.0, 7.0, 8.0, 9.0, 10, 11, 12, 13, 14, 15, 16, 17, 18, 19, 20, 25, 30, 35, 40, 45, 50, 55, 60, 65, 70, 75, 80, 85, 90, 95, 100, 110, 120, 130, 140, 150, 200, 250, 300, 350, 400, 450, 500, 550, 600, 650, 700, 750, 800, 850, 900, 950, or 1000 mg) per day for an adult human.

In certain embodiments, the compound as described herein or a pharmaceutically acceptable salt or solvate thereof, is substantially pure, in that it contains less than about 5%, or less than about 2%, or less than about 1%, or less than about 0.5%, or less than about 0.1%, of other organic small molecules, such as unreacted intermediates or synthesis by-products that are created, for example, in one or more of the steps of a synthesis method.

In various embodiments, the composition is provided in a dosage form that is suitable for oral, parenteral (e.g., intravenously, intramuscularly, subcutaneous, intraarterial), buccal, sublingual, rectal, cutaneous, nasal, vaginal, intranasal, inhalation, transdermal, ocular, intraosseous, otic, or intracranial administration route. Thus, in some embodiments, the composition dosage form is chosen from tablets, capsules, pills, powders, granulates, suspensions, emulsions, solutions, gels including hydrogels, pastes, patches, ointments, creams, plasters, drenches, osmotic delivery devices, suppositories, enemas, injectables, implants, sprays, and aerosols. The pharmaceutical compositions are formulated according to conventional pharmaceutical practice (see, e.g., Remington: The Science and Practice of Pharmacy, 20th edition, 2000, ed. A. R. Gennaro, Lippincott Williams & Wilkins, Philadelphia, and Encyclopedia of Pharmaceutical Technology, eds. J. Swarbrick and J. C. Boylan, 1988-1999, Marcel Dekker, New York).

Pharmaceutical compositions can be formulated to release the active compound immediately upon administration or at any predetermined time or time period after administration (e.g., controlled release formulations). Examples of controlled release formulations include (i) formulations that create substantially constant concentrations of the agent(s) of the disclosure within the body over an extended period of time; (ii) formulations that after a predetermined lag time create substantially constant concentrations of the agents of the disclosure within the body over an extended period of time; (iii) formulations that sustain the agent(s) action during a predetermined time period by maintaining a relatively constant, effective level of the agent(s) in the body with concomitant minimization of undesirable side effects associated with fluctuations in the plasma level of the agent(s)(sawtooth kinetic pattern); (iv) formulations that localize action of agent(s), e.g., spatial placement of a controlled release composition adjacent to or in the diseased tissue or organ; (v) formulations that achieve convenience of dosing, e.g., administering the composition once per week or once every two weeks; and (vi) formulations that target the action of the agent(s) by using carriers or chemical derivatives to deliver the compound to a particular target cell type. Administration of the compound in the form of a controlled release formulation is desirable, in some embodiments, for compounds having a narrow absorption window in the gastrointestinal tract or a relatively short biological half-life.

In some embodiments, controlled release is obtained by appropriate selection of various formulation parameters and ingredients, including, e.g., various types of controlled release compositions and coatings. In some embodiments, the compound is formulated with appropriate excipients into a pharmaceutical composition that, upon administration, releases the compound in a controlled manner. Examples include single or multiple unit tablet or capsule compositions, oil solutions, suspensions, emulsions, microcapsules, molecular complexes, microspheres, nanoparticles, patches, and liposomes.

A pharmaceutical composition comprising a compound as described herein can be administered parenterally by injection, infusion, or implantation (e.g., intraocular, subcutaneous, intravenous, intramuscular, intraperitoneal) via dosage forms, formulations, or by suitable delivery devices or implants containing conventional, non-toxic pharmaceutically acceptable carriers and adjuvants. The formulation and preparation of such compositions are well known to those skilled in the art of pharmaceutical formulation.

Suitable oral compositions as described herein include without limitation tablets, troches, lozenges, aqueous or oily suspensions, dispersible powders or granules, emulsion, hard or soft capsules, syrups or elixirs.

In another embodiment, also encompassed are pharmaceutical compositions suitable for single unit dosages that comprise a compound of the disclosure or its pharmaceutically acceptable stereoisomer, salt, or tautomer and a pharmaceutically acceptable carrier.

The compositions of the present disclosure that are suitable for oral use may be prepared according to any method known to the art for the manufacture of pharmaceutical compositions. For instance, liquid formulations of the compounds of the present disclosure contain one or more agents selected from the group consisting of sweetening agents, flavoring agents, coloring agents and preserving agents in order to provide pharmaceutically palatable preparations of a compound of the present disclosure.

For tablet compositions, a compound of the present disclosure in admixture with non-toxic pharmaceutically acceptable excipients is used for the manufacture of tablets. Examples of such excipients include without limitation inert diluents, such as calcium carbonate, sodium carbonate, lactose, calcium phosphate or sodium phosphate; granulating and disintegrating agents, for example, corn starch, or alginic acid; binding agents, for example starch, gelatin or acacia, and lubricating agents, for example magnesium stearate, stearic acid or talc. The tablets may be uncoated or they may be coated by known coating techniques to delay disintegration and absorption in the gastrointestinal tract and thereby to provide a sustained therapeutic action over a desired time period. For example, a time delay material such as glyceryl monostearate or glyceryl distearate may be employed.

Formulations for oral use may also be presented as hard gelatin capsules wherein the active ingredient is mixed with an inert solid diluent, for example, calcium carbonate, calcium phosphate or kaolin, or as soft gelatin capsules wherein the active ingredient is mixed with water or an oil medium, for example peanut oil, liquid paraffin or olive oil.

For aqueous suspensions, a compound of the present disclosure is admixed with excipients suitable for maintaining a stable suspension. Examples of such excipients include without limitation are sodium carboxymethylcellulose, methylcellulose, hydroxpropylmethylcellulose, sodium alginate, polyvinylpyrrolidone, gum tragacanth and gum acacia.

Oral suspensions can also contain dispersing or wetting agents, such as naturally-occurring phosphatide, for example, lecithin, or condensation products of an alkylene oxide with fatty acids, for example polyoxyethylene stearate, or condensation products of ethylene oxide with long chain aliphatic alcohols, for example, heptadecaethyleneoxycetanol, or condensation products of ethylene oxide with partial esters derived from fatty acids and a hexitol such as polyoxyethylene sorbitol monooleate, or condensation products of ethylene oxide with partial esters derived from fatty acids and hexitol anhydrides, for example polyethylene sorbitan monooleate. The aqueous suspensions may also contain one or more preservatives, for example ethyl, or n-propyl p-hydroxybenzoate, one or more coloring agents, one or more flavoring agents, and one or more sweetening agents, such as sucrose or saccharin.

Oily suspensions may be formulated by suspending a compound of the present disclosure in a vegetable oil, for example arachis oil, olive oil, sesame oil or coconut oil, or in a mineral oil such as liquid paraffin. The oily suspensions may contain a thickening agent, for example beeswax, hard paraffin or cetyl alcohol.

Sweetening agents such as those set forth above, and flavoring agents may be added to provide palatable oral preparations. These compositions may be preserved by the addition of an anti-oxidant such as ascorbic acid.

Dispersible powders and granules suitable for preparation of an aqueous suspension by the addition of water provide a compound of the present disclosure in admixture with a dispersing or wetting agent, suspending agent and one or more preservatives. Suitable dispersing or wetting agents and suspending agents are exemplified by those already mentioned above. Additional excipients, for example sweetening, flavoring and coloring agents, may also be present.

Pharmaceutical compositions of the present disclosure may also be in the form of oil-in-water emulsions. The oily phase may be a vegetable oil, for example olive oil or arachis oil, or a mineral oil, for example liquid paraffin or mixtures of these. Suitable emulsifying agents may be naturally-occurring gums, for example gum acacia or gum tragacanth, naturally-occurring phosphatides, for example soy bean, lecithin, and esters or partial esters derived from fatty acids and hexitol, anhydrides, for example sorbitan monoleate, and condensation reaction products of the said partial esters with ethylene oxide, for example polyoxyethylene sorbitan monoleate. The emulsions may also contain sweetening and flavoring agents.

Syrups and elixirs may be formulated with sweetening agents, for example glycerol, propylene glycol, sorbitol or sucrose. Such formulations may also contain a demulcent, a preservative, and flavoring and coloring agents. The pharmaceutical compositions may be in the form of a sterile injectable, an aqueous suspension or an oleaginous suspension. This suspension may be formulated according to the known art using those suitable dispersing or wetting agents and suspending agents which have been mentioned above. The sterile injectable preparation may also be sterile injectable solution or suspension in a non-toxic parentally acceptable diluent or solvent, for example as a solution in 1,3-butanediol. Among the acceptable vehicles and solvents that may be employed are water, Ringer's solution and isotonic sodium chloride solution. In addition, sterile, fixed oils are conventionally employed as a solvent or suspending medium. For this purpose, any bland fixed oil may be employed including synthetic mono- or diglycerides. In addition, fatty acids such as oleic acid find use in the preparation of injectables.

A compound of the present disclosure can be administered in the form of suppositories for rectal administration of the drug. These compositions can be prepared by mixing the drug with a suitable non-irritating excipient which is solid at ordinary temperatures but liquid at the rectal temperature and will therefore melt in the rectum to release the drug. Such materials are cocoa butter and polyethylene glycols.

Compositions for parenteral administrations are administered in a sterile medium. Depending on the vehicle used and concentration the concentration of the drug in the formulation, the parenteral formulation can either be a suspension or a solution containing dissolved drug. Adjuvants such as local anesthetics, preservatives and buffering agents can also be added to parenteral compositions.

In some embodiments, the composition is especially adapted for administration into or around the eye. For example, a composition can be adapted to be used as eye drops, or injected into the eye, e.g., using peribulbar or intravitreal injection. Such compositions should be sterile and substantially endotoxin-free, and within an acceptable range of pH. In some embodiments a formulation without preservatives is used. Formulation of eye medications is known in the art, see, e.g., Ocular Therapeutics and Drug Delivery: A Multi-Disciplinary Approach, Reddy, Ed. (CRC Press 1995); Kaur and Kanwar, Drug Dev Ind Pharm. 2002 May; 28 (5): 473-93; Clinical Ocular Pharmacology, Bartlett et al. (Butterworth-Heinemann; 4th edition (Mar. 15, 2001)); and Ophthalmic Drug Delivery Systems (Drugs and the Pharmaceutical Sciences: a Series of Textbooks and Monographs), Mitra (Marcel Dekker; 2nd Rev&Ex edition (Mar. 1, 2003)).

Compositions for parenteral use may be provided in unit dosage forms (e.g., in single-dose ampoules), or in vials containing several doses and in which a suitable preservative may be added (see below). The composition may be in form of a solution, a suspension, an emulsion, an infusion device, or a delivery device for implantation, or it may be presented as a dry powder to be reconstituted with water or another suitable vehicle before use. Apart from the active agent(s), the composition may include suitable parenterally acceptable carriers and/or excipients. The active agent(s) may be incorporated into microspheres, microcapsules, nanoparticles, liposomes, or the like for controlled release. Furthermore, the composition may include suspending, solubilizing, stabilizing, pH-adjusting agents, tonicity adjusting agents, and/or dispersing agents.

In some embodiments, the pharmaceutical compositions of the disclosure are in a form suitable for sterile injection. To prepare such a composition, the active agent(s) are dissolved or suspended in a parenterally acceptable liquid vehicle. Among acceptable vehicles and solvents that may be employed are water, water adjusted to a suitable pH by addition of an appropriate amount of hydrochloric acid, sodium hydroxide or a suitable buffer, 1,3-butanediol, Ringer's solution, dextrose solution, and isotonic sodium chloride solution. The aqueous formulation may also contain one or more preservatives (e.g., methyl, ethyl, or n-propyl p-hydroxybenzoate). In cases where the compound has limited solubility in water, a dissolution enhancing or solubilizing agent can be added, or the solvent may include 10-60% w/w of propylene glycol.

The pharmaceutical compositions can be administered to a subject in a single dose or in multiple doses. For example, a compound described herein can be administered once a week or for 2, 3, 4, 5, 6, 7, 8, 10, 15, 20, or more weeks. It is to be understood that, for any particular subject, specific dosage regimes should be adjusted over time according to the individual need and the professional judgment of a health care provider administering or supervising the administration of the compound. For example, the dosage of a compound can be increased if the lower dose does not provide sufficient biological activity (e.g., in the treatment of a disease or condition described herein). Conversely, the dosage of the compound can be decreased, for example, if the disease or condition is reduced or eliminated, or to reduce undesirable side-effects.

### Methods of Use and Treatment

As one advantage of the present disclosure, the compounds described herein are potent inhibitors of plasma kallikrein, that is, the compounds can reduce the activity of plasma kallikrein. In various embodiments, a compound described herein can be characterized by an inhibitory constant, IC₅₀ (half maximal inhibitory concentration), no higher than 500 nM (e.g., less than 500, 450, 400, 350, 300, 250, 200, 150, 100, 50, 10, 1, or 0.1 nM).

In an embodiment, the present disclosure provides a method for inhibiting plasma kallikrein are provided. In some embodiments, the method comprises contacting PKal with a compound of the present disclosure in an amount effective to inhibit the activity of PKal. In some embodiments, the compounds of the disclosure inhibit PKal activity with an IC₅₀ value in the range of 0.1 to 500 nM (e.g., 0.1, 0.2, 0.3, 0.4, 0.5, 0.6, 0.7, 0.8, 0.9, 1.0, 2.0, 3.0, 4.0, 5.0, 6.0, 7.0, 8.0, 9.0, 10, 11, 12, 13, 14, 15, 16, 17, 18, 19, 20, 25, 30, 35, 40, 45, 50, 55, 60, 65, 70, 75, 80, 85, 90, 95, 100, 150, 200, 250, 300, 350, 400, 450 or 500 nM). It will be understood that all ranges in the disclosure include one or both endpoints, all values in between the endpoints to one significant digit, and any subranges between the endpoints. In some embodiments, the compounds of the disclosure inhibit PKal activity with an IC₅₀ less than or equal to 500 nM, such as less than or equal to 500, 450, 400, 350, 300, 250, 200, 150, 100, 95, 90, 85, 80, 75, 70, 65, 60, 55, 50, 45, 40, 35, 30, 25, 20, 19, 18, 17, 16, 15, 14, 13, 12, 11, 10, 9, 8, 7, 6, 5, 4, 3, 2, 1, 0.9, 0.8, 0.7, 0.6, 0.5, 0.4, 0.3, 0.2, or 0.1 nM.

In some embodiments, the method comprises administering to a subject an effective amount of a compound of the disclosure or a pharmaceutically acceptable salt thereof. In some embodiments, the method inhibits PKal activity in vivo with an IC₅₀ (half maximal inhibitory concentration) value in the range of 0.1 to 500 nM (e.g., 0.1, 0.2, 0.3, 0.4, 0.5, 0.6, 0.7, 0.8, 0.9, 1.0, 2.0, 3.0, 4.0, 5.0, 6.0, 7.0, 8.0, 9.0, 10, 11, 12, 13, 14, 15, 16, 17, 18, 19, 20, 25, 30, 35, 40, 45, 50, 55, 60, 65, 70, 75, 80, 85, 90, 95, 100, 150, 200, 250, 300, 350, 400, 450 or 500 nM). In some embodiments, the method inhibits PKal activity in vivo with an IC₅₀ greater than 100 nM, such as 110, 120, 130, 140, 150, 160, 170, 180, 190, or 200 nM.

The present disclosure provides, in additional embodiments, a method for treating a subject suffering from a disease or condition in a subject. The method comprises administering an effective amount of compound or pharmaceutically acceptable salt thereof as disclosed herein, or a pharmaceutical composition thereof, to the subject. In some embodiments, the compound is selected from the group consisting of any of the compounds in Table 1, combinations thereof, and pharmaceutically acceptable salts thereof.

In various embodiments, the disease or condition is selected from the group consisting of ischemic stroke, hemorrhagic stroke, hypertension, retinopathy, diabetic retinopathy, nephropathy, cerebral edema, pulmonary hypertension, inflammation, acute myocardial infarction, deep vein thrombosis, complications from fibrinolytic treatment, stroke, angina, angioedema, sepsis, arthritis, complications of cardiopulmonary bypass, capillary leak syndrome, inflammatory bowel disease, vascular complications from diabetes, diabetic macular edema, macular degeneration, neuropathy, age related macular degeneration, retinal vein occlusions, brain edema, ischemia reperfusion injury, angiogenesis, asthma, anaphylaxis, Alzheimer's disease, Parkinson's disease, multiple sclerosis, glioblastoma multiforme, complications of fibrinolysis treatment, increased albumin excretion, macroalbuminuria, pain, amyotrophic lateral sclerosis, Creutzfeld-Jakob disease, epilepsy, brain trauma, high altitude cerebral edema, cancer, disseminated intravascular coagulation, pancreatitis, inflammation, shock, hereditary angioedema (HAE), uveitis, polyangiitis, acute respiratory distress syndrome (ARDS), thrombosis, vasculitis, Crohn's disease, ulcerative colitis, enterocolitis, arteritis, glomerulonephritis, psoriasis, endometriosis, preeclampsia, malaria, arthritis, periodic and recurrent fever, Chagas disease, Reynaud's disease, systemic sclerosis, granulomatosis with polyangiitis, small vessel vasculitis, medium vessel vasculitis, large vessel vasculitis, pan-vasculitis, systemic autoinflammatory diseases, renal insufficiency, cerebral malaria, Clarkson's disease (systemic vascular leakage syndrome), Hantavirus infection, Hantavirus renal syndrome, Hantavirus pulmonary syndrome, viral associated inflammatory disorders, retinal vasculitis, uveitis, Eales' disease, Behcet's disease, sarcoidosis, whooping cough, coronavirus infection, and non-infectious posterior uveitis.

In some embodiments, the subject is an animal, such as a human or non-human animal (e.g., a mammal), and is used interchangeably with “patient” when the subject is undergoing medical treatment by a health care provider.

### Combination Therapy

The disclosure also provides combination pharmaceutical compositions comprising: (a) at least one compound or a pharmaceutically acceptable salt thereof as disclosed herein, and (b) at least one inhibitor of inflammation, pain, or edema. In some embodiments, orally delivered pharmaceutical compositions comprising the combination pharmaceutical compositions of the disclosure are provided. The disclosure further provides tablets, capsules, orally delivered particles, injectable suspensions and solutions, and compositions for pulmonary or nasal delivery comprising the combination pharmaceutical composition.

### Kits

In another embodiment, the disclosure provides a kit comprising a compound or a pharmaceutically acceptable salt thereof as described herein. The kit also comprises instructions to a health care provider for administering the compound to a patient.

### EXAMPLES

The following examples provide further embodiments of the present disclosure. The examples are illustrative and non-limiting. Although essentially any compositions, compounds, and methods similar to those described herein can be used in the practice or testing of the present disclosure, only exemplary compositions, compounds, and methods are described.

### Synthesis of Compounds

As depicted in the Examples below, in certain exemplary embodiments, compounds are prepared according to the following general procedures. It will be appreciated that, although the general methods depict the synthesis of certain compounds of the present invention, the following general methods, and other methods known to one of ordinary skill in the art, can be applied to all compounds and subclasses and species of each of these compounds, as described herein. Additional compounds of the invention are prepared by methods substantially similar to those described herein in the Examples known to one skilled in the art.

### Preparation of Intermediate Compounds

### Example 1A: Preparation of ethyl 1-(4-(2-cyanopropan-2-yl)benzyl)-1H-pyrazole-4-carboxylate (7_Int-5)

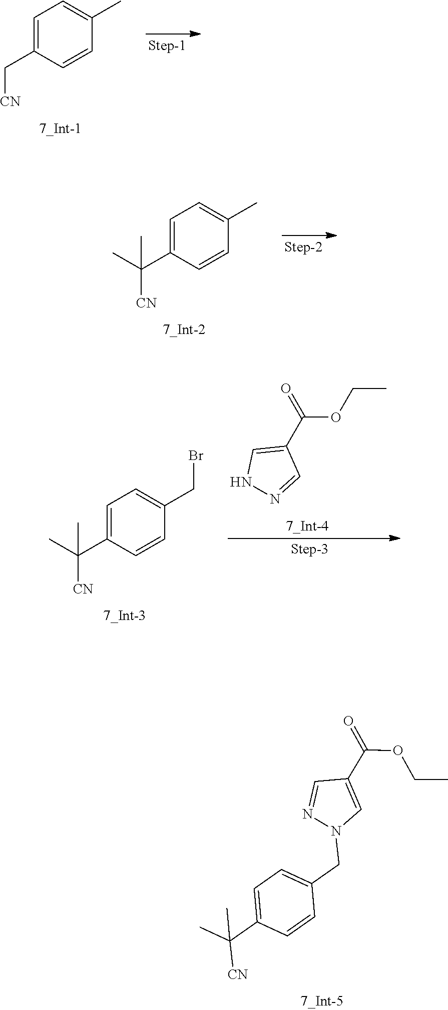

### Step-1. Synthesis of 2-methyl-2-(p-tolyl) propanenitrile (7_Int-2)

A solution of 2-(p-tolyl) acetonitrile (200 g, 1524 mmol, 1.0 eq), in N-Methyl-2-pyrrolidone (1000 mL, 5V) and Tetrahydrofuran (1000 mL, 5V) was added Sodium tert-butoxide (586.07 g, 6098 mmol, 4.0 eq) at 0° C. to 5° C. portion wise under nitrogen atmosphere. The reaction mixture (RM) was stirred at 0° C. to 5° C. for 30 min. Iodomethane (865.62 g, 6098 mmol, 4.0 eq), was added drop wise to the RM at 0° C. to 5° C. The RM was stirred at 0° C. to RT for 2.5 h. After completion of reaction, the RM was transferred into deionized water (400 mL) and product was extracted with ethyl acetate (2000 mL). The combined organic fractions were washed by cold water (4×500 mL) to remove N-Methyl-2-pyrrolidone, concentrated and then purified by column chromatography (2-5% EtOAc in Hexanes) to give compound 7_Int-2. ¹H NMR (400 MHz, DMSO-d6) δ 1.651 (s, 6H), 2.298 (s, 3H), 7.226 d, J=8 Hz, 2H), 7.392 (d, J=6.8 Hz, 2H).

### Step-2. Synthesis of 2-(4-(bromomethyl)phenyl)-2-methylpropanenitrile (7_Int-3)

A 10000 mL 4N Round bottom flask (RBF) attached to mechanical stirrer and condenser was charged with 2-methyl-2-(p-tolyl) propanenitrile (170 g, 1067 mmol, 1.0 eq) and Carbon tetrachloride (3400 mL, 20V) at RT. AIBN (17.53 g, 106 mmol, 0.1 eq), was added to the RM. N-Bromosuccinimide (209.04 g, 1174 mmol, 1.1 eq) was added portion wise to the RM at RT. Resulting RM was heated to 90° C. and stirred for 2 h. Note: After completion of reaction, the reaction mixture was cooled to RT. The RM was quenched into DM water (3400 mL) and product was extracted with DCM (2×2000 mL). The combined organic fractions were concentrated and then purified by column chromatography (7% EtOAc in Hexanes) to give compound (7_Int-3). ¹H NMR (400 MHz, DMSO-d6) δ 1.654 (s, 6H), 4.714 (s, 2H), 7.507 (s, 4H).

### Step-3. Synthesis of ethyl 1-(4-(2-cyanopropan-2-yl)benzyl)-1H-pyrazole-4-carboxylate (7_Int-5)

A 3000 mL 4N RBF attached to mechanical stirrer and condenser was charged with ethyl 1H-pyrazole-4-carboxylate (70 g, 499 mmol, 1.0 eq), 2-(4-(bromomethyl)phenyl)-2-methylpropanenitrile (130.84 g, 549 mmol, 1.1 eq) and Acetone (700 mL, 10V) at RT. Cs₂CO₃ (390.8 g, 1198 mmol, 2.4 eq) was added to RM. The RM was heated to 60-65° C. and stirred for 6 h. After completion of reaction, the reaction mixture was cooled to RT. The RM was filtered to remove Cs₂CO₃; washed with EtOAc. Filtrate was concentrated and then purified by column chromatography (15% Ethylacetate in hexane) to give compound (7_Int-5). ¹H NMR (400 MHz, DMSO-d6) δ 1.656 (s, 6H), 4.204 (q, J=6.8 Hz, 2H), 5.372 (s, 2H), 7.328 (d, J=8 Hz, 2H), 7.498 (d, J=8 Hz, 2H), 8.053 (s, 1H), 8.482 (s, 1H).

### Example 2A: Preparation of ethyl 1-(4-(cyanomethyl)benzyl)-1H-pyrazole-4-carboxylate (1_Int-4)

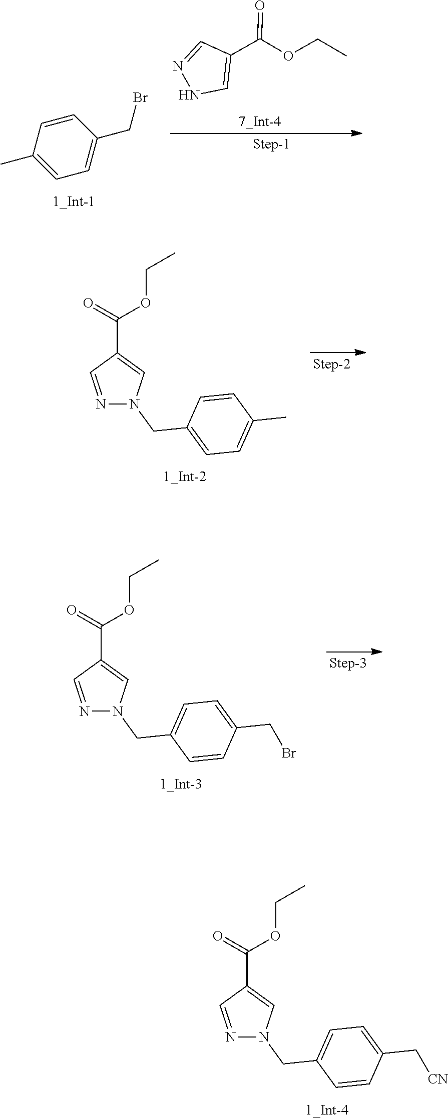

### Step-1. Synthesis of ethyl 1-(4-methylbenzyl)-1H-pyrazole-4-carboxylate (1_Int-2)

A 10000 mL 4N RBF attached to mechanical stirrer and condenser was charged with ethyl 1H-pyrazole-4-carboxylate (250 g, 1783 mmol, 1.0 eq), 1-(bromomethyl)-4-methylbenzene (363.1 g, 1962 mmol, 1.1 eq) and Acetone (6250 mL, 25V) at RT. Cs₂CO₃ (390.8 g, 1198 mmol, 2.4 eq) was added to RM. The RM was heated to 60-65° C. and stirred for 16 h. After completion of reaction, the reaction mixture was cooled to RT. The RM was filtered to remove Cs₂CO₃; washed with EtOAc; filtrate was concentrated under reduced pressure to obtain crude. The crude was purified by trituration in Hexanes Hexanes to give compound (1_Int-2). ¹H NMR (400 MHz, DMSO-d6) δ 1.227 (t, J=6.8 Hz, 3H), 2.247 (s, 3H), 4.176 (q, J=6.8 Hz, 2H), 5.282 (s, 2H), 7.144-7.136 (m, 4H), 7.828 (s, 1H), 8.390 (s, 1H).

### Step-2. Synthesis of ethyl 1-(4-(bromomethyl)benzyl)-1H-pyrazole-4-carboxylate (1_Int-3)

A 10000 mL 4N RBF attached to mechanical stirrer and condenser was charged with ethyl 1-(4-methylbenzyl)-1H-pyrazole-4-carboxylate (300 g, 1228 mmol, 1.0 eq) and 1,2 dichloro ethane (6000 mL, 20V) at RT. Benzoyl peroxide (29.71 g, 122.8 mmol, 0.1 eq) was added to the RM. N-Bromosuccinimide (240.4 g, 1350 mmol, 1.1 eq) was added portion wise to the RM at RT. Resulting RM was heated to 90° C. and stirred for 4 h. After completion of reaction, the reaction mixture was cooled to RT. RM was diluted with DCM (2000 mL). The RM was Washed with (2×4000 ml) Sat. Na₂SO₄ solution and concentrated under reduced pressure to obtain crude. The crude was purified by column chromatography (17-25% EtOAc in Hexanes) to give compound (1_Int-3). ¹H NMR (400 MHz, DMSO-d6) δ 1.270-1.235 (m, 3H), 4.207 (q, J=6.8 Hz, 2H), 4.682 (s, 2H), 5.364 (s, 2H), 7.243 (d, J=8.4 Hz, 4H), 7.421 (d, J=8.4 Hz, 2H), 7.868 (s, 1H), 8.478 (s, 1H).

### Step-3. Synthesis of ethyl 1-(4-(cyanomethyl)benzyl)-1H-pyrazole-4-carboxylate (1_Int-4)

A 5000 mL 4N RBF attached to mechanical stirrer and condenser was charged with ethyl 1-(4-(bromomethyl)benzyl)-1H-pyrazole-4-carboxylate (242 g, 499748 mmol, 1.0 eq), ACN (2420 mL, 10V) and Cs₂CO₃ (488 g, 1497 mmol, 2.0 eq) at RT followed by slowly addition of TMSCN (334 g, 3369 mmol, 4.5 eq) at RT. The RM was heated to 80-85° C. and stirred for 16 h. After completion of reaction, the reaction mixture was cooled to RT. The RM was filtered to remove Cs₂CO₃; washed with EtOAc. Solids residue was discarded and filtrate was concentrated under reduced pressure to obtain crude. The crude was purified by column chromatography (12-15% EtOAc in Hexanes) to give compound (1_Int-4). ¹H NMR (400 MHz, DMSO-d6) δ 1.282-1.234 (m, 3H), 4.016 (s, 2H), 4.20 (q, J=7.2 Hz, 2H), 5.363 (s, 2H), 7.339-7.280 (m, 4H), 8.051 (s, 1H), 8.468 (s, 1H).

### Example 3A: Preparation of ethyl 1-(4-((3-cyano-1H-pyrrol-1-yl)methyl)benzyl)-1H-pyrazole-4-carboxylate (6_Int-1)

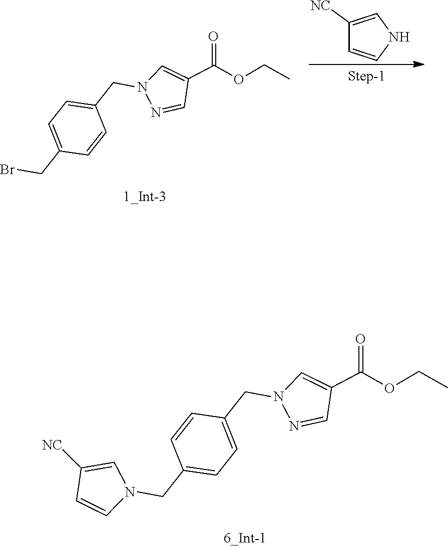 [SMILES: CCOC(=O)c1cnn(Cc2ccc(CBr)cc2)c1 ; N#Cc1cc[nH]c1 ; CCOC(=O)c1cnn(Cc2ccc(Cn3ccc(C#N)c3)cc2)c1]

### Step-1. Synthesis of ethyl 1-(4-((3-cyano-1H-pyrrol-1-yl)methyl)benzyl)-1H-pyrazole-4-carboxylate (6_Int-1)

A stirred solution of 1H-pyrrole-3-carbonitrile (0.2 g, 2.16 mmol, 1.0 eq) in DMF (2 mL, 10V) was prepared and NaH (60% in mineral oil)(0.156 g, 3.24 mmol, 1.5 eq) was added at 0° C. The RM was stirred at 0° C. for 30 min. Ethyl 1-(4-(bromomethyl)benzyl)-1H-pyrazole-4-carboxylate (1_Int-3)(0.772 g, 2.39 mmol, 1.1 eq) was added to RM at 0° C. The RM was warmed to RT and stirred for 3 h. After completion of the reaction, the RM was quenched in water and extracted by ethyl acetate. The combined organic fractions were dried over Na₂SO₄, concentrated and then purified by column chromatography (20% ethyl acetate in hexanes) to give compound (6_Int-1). MS(ES): 335.30 m/z [M+H]+, LCMS purity: 95.09%, ¹H NMR (400 MHz, DMSO-d6) δ 1.246 (t, J=8 Hz, 3H), 4.194 (q, J=7.2 Hz, 2H), 5.336 (s, 2H), 6.987 (t, J=2.4 Hz, 1H), 7.267-7.208 (m, 4H), 7.699 (t, J=2 Hz, 1H), 7.845 (s, 1H), 8.448 (s, 1H).

### Example 4A: Preparation of 4-(aminomethyl)-2-fluorobenzimidamidedihydrochloride (43_Int-5)

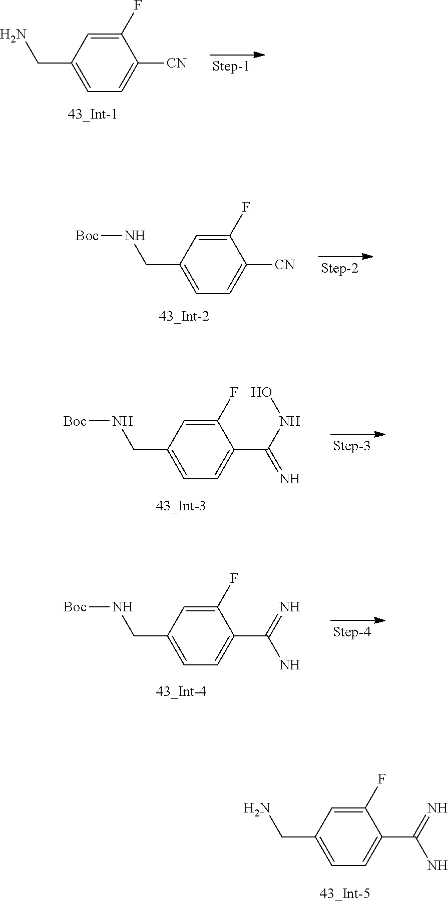 [SMILES: N#Cc1ccc(CN)cc1F ; CC(C)(C)OC(=O)NCc1ccc(C#N)c(F)c1 ; CC(C)(C)OC(=O)NCc1ccc(C(=N)NO)c(F)c1 ; CC(C)(C)OC(=O)NCc1ccc(C(=N)N)c(F)c1 ; N=C(N)c1ccc(CN)cc1F]

### Step-1. Synthesis of tert-butyl (4-cyano-2-fluorobenzyl) carbamate (43_Int-2)

A stirred solution of 4-(aminomethyl)-2-fluorobenzonitrile (43_Int-1)(3 g, 19.97 mmol, 1.0 eq) in 1,4 dioxane (36 mL, 12V) and 2N NaOH (18 mL, 6V) was prepared and Boc anhydride (4.79 g, 21.97 mmol, 1.1 eq) was added at 0° C. The reaction mixture was stirred at RT for 3 h. After completion of the reaction, the RM was quenched in water and extracted by ethyl acetate. The combined organic fractions were dried over Na₂SO₄; concentrated to give compound (43_Int-2). MS(ES): 251.00.m/z [M+H]+, LCMS purity: 100%, 1H NMR (400 MHz, DMSO-d6) δ 1.393 (s, 9H), 4.216 (d, J=5.2 Hz, 2H), 7.281 (t, J=7.5 Hz, 2H), 7.551 (s, 1H), 7.885 (t, J=8 Hz, 1H)

### Step-2. Synthesis of tert-butyl (3-fluoro-4-(N-hydroxycarbamimidoyl)benzyl) carbamate (43_Int-3)

A stirred solution of tert-butyl (4-cyano-3-fluorobenzyl) carbamate (43_Int-2)(3.4 g, 13.58 mmol, 1.0 eq) and methanol (34 mL, 10V) was prepared and hydroxylamine hydrochloride (1.604 g, 23.09 mmol, 1.7 eq) and DIPEA (3.95 g, 23.09 mmol, 1.7 eq) were added at RT. RM was stirred at 70° C. for 16 h. After completion of the reaction, the RM was evaporated, and the residue was quenched in water and extracted by 10% methanol in DCM. The combined organic fractions were dried over Na₂SO₄; concentrated to give compound (43_Int-3). MS(ES): 284.10 m/z [M+H]+LCMS purity: 81.45%, ¹H NMR (400 MHz, DMSO-d6) δ 1.393 (s, 9H), 4.133 (d, J=5.2 Hz, 2H), 7.101-70.31 (m, 2H), 7.125 (s, 1H), 7.483-7.417 (m, 2H), 7.611 (t, J=8 Hz, 1H), 9.593 (s, 1H).

### Step-3. Synthesis of tert-butyl (4-carbamimidoyl-3-fluorobenzyl) carbamate (43_Int-4)

A stirred solution of tert-butyl (3-fluoro-4-(N-hydroxycarbamimidoyl)benzyl) carbamate (43_Int-3)(3.5 g, 12.35 mmol, 1.0 eq) and methanol (35 mL, 10V) was prepared and ammonium formate (2.34 g, 37.06 mmol, 3.0 eq) and 10% Pd/C (0175 g, 0.065% w/w) was added at RT. The RM was stirred at 70° C. for 16 h under 20 kg H2 pressure in autoclave. After completion of the reaction, the RM was filtered through celite bed, filtrate was concentrated to give compound (43_Int-4). MS(ES): 267.80 m/z [M+H]+LCMS purity: 96.46%, ¹H NMR (400 MHz, DMSO-d6) δ 1.394 (s, 9H), 4.195 (d, J=5.6 Hz, 2H), 7.244 (d, J=8 Hz, 2H), 7.254-7.233 (m, 3H), 7.639-7.580 (m, 3H), 8.415 (s, 1H).

### Step-4. Synthesis of 4-(aminomethyl)-2-fluorobenzimidamide dihydrochloride (43_Int-5)

A stirred solution of tert-butyl (4-carbamimidoyl-3-fluorobenzyl) carbamate (43_Int-4)(4.8 g, 16.83 mmol, 1.0 eq) and water (52 mL, 11V) was prepared and concentrated HCl (16 mL, 3.3V) was added at RT. The RM was stirred at rt for 3 h. After completion of the reaction, the RM was concentrated and triturated by methanol to give compound (43_Int-5). MS(ES): 168.13 m/z [M+H]+, ¹H NMR (400 MHz, DMSO-d6) δ 4.141 (d, J=4 Hz, 2H), 7.658-7.155 (m, 3H), 7.737-7.686 (m, 2H), 8.772 (s, 1H), 9.564-9.440 (m, 2H).

### Example 5A: Preparation of 4-(aminomethyl)-3-fluorobenzimidamide dihydrochloride (45_Int-5)

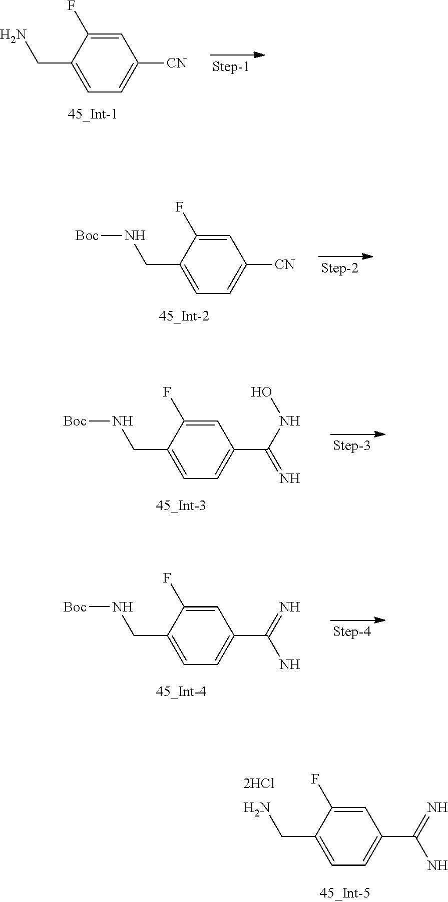 [SMILES: N#Cc1ccc(CN)c(F)c1 ; CC(C)(C)OC(=O)NCc1ccc(C#N)cc1F ; CC(C)(C)OC(=O)NCc1ccc(C(=N)NO)cc1F ; CC(C)(C)OC(=O)NCc1ccc(C(=N)N)cc1F ; N=C(N)c1ccc(CN)c(F)c1]

### Step-1. Synthesis of tert-butyl (4-cyano-2-fluorobenzyl) carbamate (45_Int-2)

A stirred solution of 4-(aminomethyl)-3-fluorobenzonitrile (45_Int-1)(1 g, 6.65 mmol, 1.0 eq) in 1,4 dioxane (12 mL, 12V) and 2N NaOH (6 mL, 6V) was prepared and Boc anhydride (1.59 g, 7.32 mmol, 1.1 eq) was added at 0° C. The reaction mixture was stirred at RT for 3 h. After completion of the reaction, the RM was quenched in water and extracted by ethyl acetate. The combined organic fractions were dried over Na₂SO₄; concentrated to give compound (45_Int-2). ¹H NMR (400 MHz, DMSO-d6) δ 1.146 (s, 9H), 4.220 (d, J=5.2 Hz, 2H), 7.442 (t, J=7.5 Hz, 2H), 7.525 (s, 1H), 7.812 (t, J=8 Hz, 2H).

### Step-2. Synthesis of tert-butyl(2-fluoro-4-(N-hydroxycarbamimidoyl)benzyl) carbamate (45_Int-3)

A stirred solution of tert-butyl (4-cyano-2-fluorobenzyl) carbamate (45_Int-2)(5.3 g, 21.17 mmol, 1.0 eq) and methanol (53 mL, 10V) was prepared and hydroxylamine hydrochloride (2.5 g, 36.0 mmol, 1.7 eq) and DIPEA (4.64 g, 36.0 mmol, 1.7 eq) were added at RT. The RM was stirred at 70° C. for 16 h. After completion of the reaction, the RM was evaporated, residue was quenched in water and extracted by 10% methanol in DCM. The combined organic fractions were dried over Na₂SO₄; concentrated to give compound (45_Int-3). MS(ES): 284.21 m/z [M+H]+

### Step-3. Synthesis of tert-butyl (4-carbamimidoyl-2-fluorobenzyl) carbamate (45_Int-4)

A stirred solution of tert-butyl(2-fluoro-4-(N-hydroxycarbamimidoyl)benzyl) carbamate (45_Int-3)(2.4 g, 8.47 mmol, 1.0 eq) and methanol (24 mL, 10V) was prepared and ammonium formate (1.6 g, 25.41 mmol, 3.0 eq) and 10% Pd/C (0.16 g, 0.065% w/w) at RT. The RM was stirred at 70° C. for 16 h under 20 kg H2 pressure in autoclave. After completion of the reaction, the RM was filtered through celite bed, filtrate was concentrated to give compound (45_Int-4). MS(ES): 268.18 m/z [M+H]+

### Step-4. Synthesis of 4-(aminomethyl)-3-fluorobenzimidamide dihydrochloride (45_Int-5)

A stirred solution of tert-butyl (4-carbamimidoyl-2-fluorobenzyl) carbamate (45_Int-4)(2.0 g, 7.48 mmol, 1.0 eq) and water (22 mL, 11V) was prepared and concentrated HCl (6.6 mL, 3.3V) was added at RT. The RM was stirred at RT for 3 h. After completion of the reaction, the RM was concentrated and triturated by methanol to give compound (45_Int-5). MS(ES): 168.13 m/z [M+H]+, ¹H NMR (400 MHz, DMSO-d6) δ 4.145 (s, 2H), 7.210 (s, 1H), 7.336 (s, 1H), 7.463 (s, 1H), 7.878-7.765 (m, 2H), 8.816 (s, 2H), 9.442 (s, 1H), 9.634 (s, 1H).

### Example 6A: Preparation of ethyl 1-(4-(3-cyanobenzyl)benzyl)-1H-pyrazole-4-carboxylate (28_Int-4)

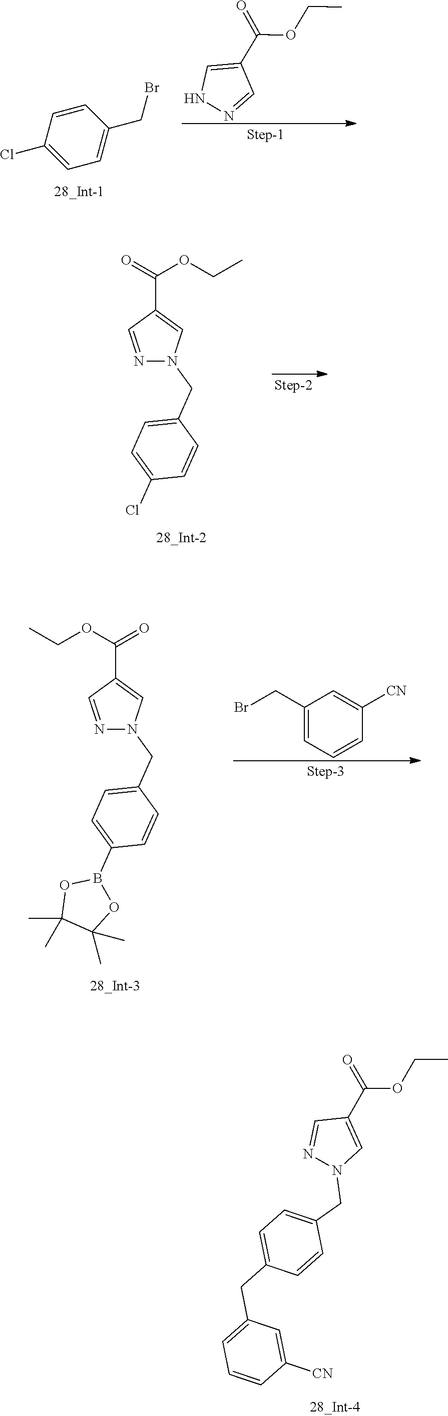

### Step-1. Synthesis of ethyl 1-(4-chlorobenzyl)-1H-pyrazole-4-carboxylate (28_Int-2)

A stirred solution of ethyl 1H-pyrazole-4-carboxylate (1 g, 7.14 mmol, 1 eq) and 1-(bromomethyl)-4-chlorobenzene (28_int-1)(1.5 g, 7.85 mmol, 1.1 eq) in Acetone (10 mL, 10V) was prepared and Cs₂CO₃ (5.53 g, 17.14 mmol, 2.4 eq) was added at RT. The RM was heated to 65° C. and stirred for 4 h. After completion of the reaction, the RM was cooled at RT, quenched in water and extracted by ethyl acetate. The combined organic fractions were dried over Na₂SO₄; concentrated and then purified by flash column chromatography (50% ethyl acetate in hexanes) to give compound (28_Int-2). MS(ES): 265.30 m/z [M+], 266.30 [M+2]+, LCMS purity: 63%, ¹H NMR (400 MHz, DMSO-d6) δ 400 MHz, DMSO-d6: δ 1.26 (t, J=6.8 Hz, 3H), 4.28-4.210 (m, 2H), 5.38 (s, 2H), 7.30 (d, J=8.40 Hz, 2H), 7.43 (d, J=8.40 Hz, 2H), 7.88 (s, 1H), 8.49 (s, 1H).

### Step-2. Synthesis of ethyl 1-(4-(4,4,5,5-tetramethyl-1,3,2-dioxaborolan-2-yl)benzyl)-1H-pyrazole-4-carboxylate (28_Int-3)

A stirred solution of ethyl 1-(4-chlorobenzyl)-1H-pyrazole-4-carboxylate (28_Int-2)(1.0 g, 3.78 mmol, 1.0 eq) in 1,4 dioxane (5 ml, 5V) was prepared and Bis(pinacolato)diboron (1.14 g, 4.54 mmol, 1.2 eq), KOAc (1.11 g, 11.36 mmol, 3 eq), XPhosPdG2 (0.350 g, 0.37 mmol, 0.1 eq) and water (0.5 ml, 0.5V) were added at RT. The RM was heated to 100° C. and stirred for 16 h. After completion of the reaction, the RM was cooled to RT, quenched in water and extracted by ethyl acetate. The combined organic fractions were dried over Na₂SO₄; concentrated and then purified by flash column chromatography (20% ethyl acetate in hexanes) to give compound (28_Int-3). MS(ES): 357.51 m/z [M+1]+, LCMS purity: 70%, ¹H NMR (400 MHz, DMSO-d6) δ 1.26 (s, 12H), 1.26 (t, 3H), 4.228-4.210 (m, 2H), 5.38 (d, J=12.80 Hz, 2H), 7.25 (d, J=8.00 Hz, 2H), 7.65 (d, J=8.00 Hz, 1H), 7.87 (s, 1H), 0.00 (s, 1H), 8.47 (s, 1H).

### Step-3. Synthesis of ethyl 1-(4-(3-cyanobenzyl)benzyl)-1H-pyrazole-4-carboxylate (28_Int-4)

A stirred solution of ethyl 1-(4-(4,4,5,5-tetramethyl-1,3,2-dioxaborolan-2-yl)benzyl)-1H-pyrazole-4-carboxylate (28_Int-3)(0.300 g, 0.84 mmol, 1.0 eq) in 1,4 dioxane (3 ml, 10V) was prepared and 3-(bromomethyl)benzonitrile (0.181 g, 0.92 mmol, 1.1 eq), K₃PO₄ (0.535 g, 3.51 mmol, 3 eq), Pd (dppf) C₁₂, DCM (0.068 g, 0.11 mol, 0.1 eq) and water (1.5 ml, 5V) were added at RT. The RM was heated to 100° C. and stirred for 6 h. After completion of the reaction, the RM was cooled to RT, quenched in water and extracted by ethyl acetate. The combined organic fractions were dried over Na₂SO₄; concentrated and then purified by flash column chromatography (30% ethyl acetate in hexanes) to give compound (28_Int-4). MS(ES): 346.39 m/z [M+1]+, LCMS purity: 100%, ¹H NMR (400 MHz, DMSO-d6) δ 1.22 (s, 3H), 3.98 (s, 2H), 4.20 (q, J=6.8 Hz, 2H), 5.31 (s, 2H), 7.254-7.193 (m, 4H), 7.49 (t, J=8.00 Hz, 1H), 7.56 (d, J=8.00 Hz, 1H), 0.00 (d, J=7.60 Hz, 1H), 7.72 (s, 1H), 7.84 (s, 1H), 8.434 (s, 1H).

### Example 7A: Preparation of 4-(aminomethyl)-2,6-difluorobenzimidamide dihydrochloride (44_Int-8)

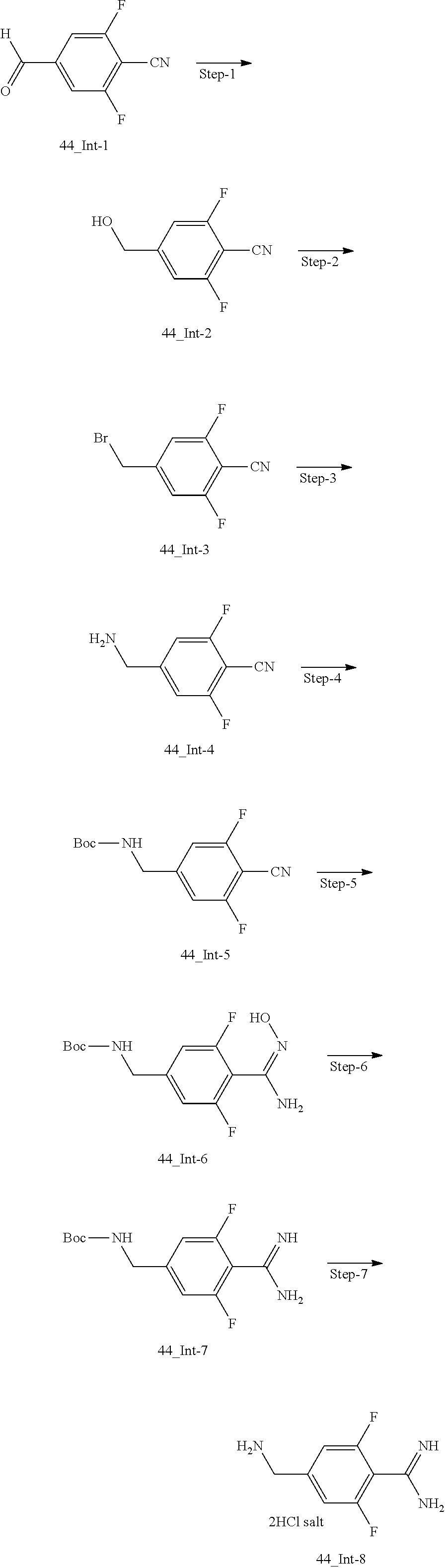

### Step-1. Synthesis of 2,6-difluoro-4-(hydroxymethyl)benzonitrile (44_Int-2)

A stirred solution of 2,6-difluoro-4-formylbenzonitrile (44_Int-1) (8 g, 0.047 mmol, 1.0 eq) in methanol (160 mL, 20V) was prepared and NaBH₄ (2.21 g, 0.047 mmol, 1 eq) was added at 0° C. The RM was stirred at RT for 1 h. After completion of the reaction, the RM was quenched in water and extracted by DCM. The combined organic fractions were dried over Na₂SO₄; concentrated to give compound (44_Int-2). ¹H NMR (400 MHz, DMSO-d6) δ 4.60 (d, J=8 Hz, 2H), 5.72 (t, J=9.1 Hz, 1H), 7.34 (d, J=8 Hz, 2H).

### Step-2. Synthesis of 2,6-difluoro-4-(hydroxymethyl)benzonitrile (44_Int-3)

A stirred solution of 2,6-difluoro-4-(hydroxymethyl)benzonitrile (44_Int-2)(7.5 g, 0.0443 mmol, 1.0 eq) in MTBE (75 mL, 10V) was prepared and PBr₃ (14.37 g, 0.0532 mmol, 1.2 eq) was added at 0° C. The RM was stirred at RT for 2 h. After completion of the reaction, the RM was quenched in cold water and extracted by ethyl acetate. The combined organic fractions were dried over Na₂SO₄; concentrated to give compound (44_Int-3). ¹H NMR (400 MHz, DMSO-d6) δ 4.721 (s, 2H), 7.564 (d, J=8.8 Hz, 2H).

### Step-3. Synthesis of 4-(aminomethyl)-2,6-difluorobenzonitrile (44_Int-4)

Ammonia gas was purged in methanol for 30 min at 0° C. A solution of 2,6-difluoro-4-(hydroxymethyl)benzonitrile (44_Int-3)(9.0 g, 0.06 mmol, 1.0 eq) in MeOH (40 mL, 10V) was added to the RM and was stirred at 0° C. for 4 h. After completion of the reaction, the RM was concentrated to give compound (44_Int-4). MS(ES): 169 m/z [M+1]+, LCMS purity: 84%.

### Step-4. Synthesis of tert-butyl (4-cyano-3,5-difluorobenzyl) carbamate (44_Int-5)

A stirred solution of 4-(aminomethyl)-2,6-difluorobenzonitrile (44_Int-4)(10 g, 59.5238 mmol, 1.0 eq) in 1,4 dioxane (120 mL, 12V) was prepared. 2N NaOH (60 ml, 6V) and Boc-anhydride (14.27 μm, 0.065 mmol, 1.1 eq) were added at RT and stirred for 5 h. After completion of the reaction, the RM was quenched in cold water and extracted by ethyl acetate. The combined organic fractions were dried over Na₂SO₄; concentrated to give compound (44_Int-5)₁H NMR (400 MHz, DMSO-d6) δ 1.39 (s, 9H), 4.52 (s, 2H), 7.27 (d, J=8.8 Hz, 2H).

### Step-5. Synthesis of tert-butyl (3,5-difluoro-4-(N-hydroxycarbamimidoyl)benzyl) carbamate (44_Int-6)

A stirred solution of tert-butyl (4-cyano-3,5-difluorobenzyl) carbamate (44_Int-5)(6 g, 22.388 mmol, 1.0 eq) in MeOH (60 mL, 10V) was prepared. Hydroxylamine hydrochloride (2.6 μm, 38.0596 mmol, 1.7 eq) and DIPEA (6.5 ml, 38.0596 mmol, 1.7 eq) were added at RT and stirred at 70° C. for 16 h. After completion of the reaction, the RM was cooled to RT. The RM was then quenched in water and extracted by DCM. The combined organic fractions were dried over Na₂SO₄; concentrated to give compound (44_Int-6). MS(ES): 302 m/z [M+1]+, LCMS purity: 65%.

### Step-6. Synthesis of tert-butyl (4-carbamimidoyl-3,5-difluorobenzyl) carbamate (44_Int-7)

A stirred solution of tert-butyl (3,5-difluoro-4-(N-hydroxycarbamimidoyl)benzyl) carbamate (44_Int-6)(6 g, 19.933 mmol, 1.0 eq) in MeOH (60 mL, 10V) was prepared. Ammonium chloride (5.38 g, 99.66 mmol, 5.0 eq) and iron (5.40 g, 99.66 mmol, 5.0 eq) were added at RT. The RM was cooled to 0° C. followed by dropwise addition of acetic acid (30 mL, 5V). The RM was then heated to 70° C. and stirred for 16 h. After completion of the reaction, the RM was cooled to RT. The RM was concentrated and quenched in cold water and slowly basified to pH ~10. The solids were isolated via filtration and extracted by DCM. The combined organic fractions were dried over Na₂SO₄; concentrated to give compound (44_Int-7). MS(ES): 286 m/z [M+1]+, LCMS purity: 65%.

### Step-7. Synthesis of 4-(aminomethyl)-2,6-difluorobenzimidamide (44_Int-8)

A stirred solution of tert-butyl (4-carbamimidoyl-3,5-difluorobenzyl) carbamate (44_Int-7)(2.2 g, 25.473 mmol, 1.0 eq) was prepared in water (24 mL, 11V) and concentrated HCl (7.26 mL, 3.3V) was added at RT and stirred for 3 h. After completion of the reaction, the RM was concentrated and triturated in methanol to give compound (44_Int-8). MS(ES): 231 m/z [M+1]+, LCMS purity: 67%.

### Example 9A: Preparation of methyl 1-(4-(2-cyanopropan-2-yl)benzyl)-5-(methoxymethyl)-1H-pyrazole-4-carboxylate (82_Int-3)

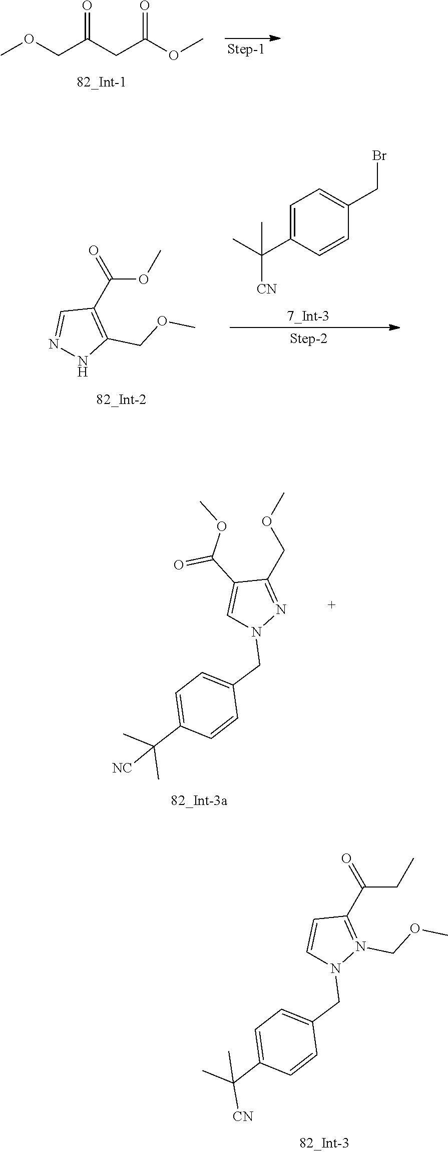

### Step-1. Synthesis of methyl 5-(methoxymethyl)-1H-pyrazole-4-carboxylate (82_Int-2)

A solution of methyl 4-methoxy-3-oxobutanoate (82_Int-1)(15 g, 102 mmol, 1.0 eq) and Dimethylformamide Dimethyl Acetal (12.23 g, 102 mmol, 1.0 eq) was prepared 110° C. and stirred for 1 h. The RM was cooled to RT followed by addition of ethanol (150 mL, 10V) and hydrazine hydrate (99%) (5.13 g, 102 mmol, 1.0 eq). The RM was heated to 70° C. and stirred for 2 h. After completion of the reaction, the RM was evaporated and then purified by column chromatography (25-30% ethyl acetate in hexanes) to give compound (82_Int-2). ¹H NMR (400 MHz, DMSO-d6) δ 3.258 (s, 3H), 3.758 (s, 3H), 4.702 (s, 2H), 7.837 (s, 1H), 8.281 (s, 1H).

### Step-2. Synthesis of methyl 1-(4-(2-cyanopropan-2-yl)benzyl)-5-(methoxymethyl)-1H-pyrazole-4-carboxylate (82_Int-3)

A stirred solution of methyl 5-(methoxymethyl)-1H-pyrazole-4-carboxylate (82_Int-2)(1.0 g, 5.87 mmol, 1.0 eq) was prepared DMF (10 mL, 10V) and NaHMDS (1M in THF)(5.8 mL, 5.87 mmol, 1.0 eq) was added at 0° C. and stirred for 30 min. 2-(4-(bromomethyl)phenyl)-2-methylpropanenitrile (7_Int-3)(1.39 g, 5.87 mmol, 1.0 eq) was then added to the RM at 0° C. and stirred for an additional 16 h. After completion of the reaction, the RM was quenched in water and extracted with ethyl acetate. The combined organic fractions were dried over Na₂SO₄; concentrated and then purified by flash column chromatography (15% ethyl acetate in hexanes)(82_Int-3). Confirmed by 2D NMR (ROE analysis). MS(ES): 328 m/z, LCMS purity: 99.11%, 1H 400 MHz, DMSO-d6: δ 1.654 (s, 6H), 3.258 (s, 3H), 3.758 (s, 3H), 4.797 (s, 2H), 5.394 (s, 2H), 7.258 (d, J=8 Hz, 2H), 7.478 (d, J=8 Hz, 2H), 7.903 (s, 1H).

### Example 10A: Preparation of methyl 1-(4-(cyanomethyl)benzyl)-5-(methoxymethyl)-1H-pyrazole-4-carboxylate (85_Int-3)

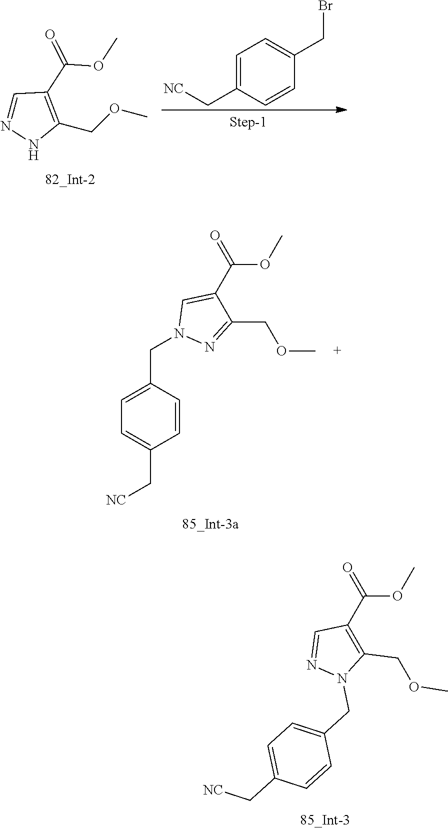

### Step-1. Synthesis of methyl 1-(4-(cyanomethyl)benzyl)-5-(methoxymethyl)-1H-pyrazole-4-carboxylate (85_Int-3)

A stirred solution of methyl 5-(methoxymethyl)-1H-pyrazole-4-carboxylate (82_Int-2)(1.0 g, 5.87 mmol, 1.0 eq) was prepared in DMF (10 mL, 10V) and NaHMDS (1M in THF)(5.8 mL, 5.87 mmol, 1.0 eq) was added at 0° C. and stirred for 30 min. 2-(4-(bromomethyl)phenyl) acetonitrile (1.4 g, 7.05 mmol, 1.2 eq) was then added to the RM at 0° C. and stirred at RT for an additional 16 h. After completion of the reaction, the RM was quenched in water and extracted with ethyl acetate. The combined organic fractions were dried over Na₂SO₄; concentrated and then purified by flash column chromatography (25-30% ethyl acetate in hexanes)(85_Int-3). Confirmed by 2D NMR (ROE analysis). MS(ES): 300 m/z, 1H 400 MHz, DMSO-d6: δ 3.233 (s, 3H), 3.745 (s, 3H), 4.029 (s, 2H), 4.514 (s, 2H), 5.352 (s, 2H), 7.356-7.301 (m, 4H), 8.046 (s, 1H).

### Example 11A: Preparation of ethyl 1-(4-(2-(dimethylamino)-2-oxoethyl)benzyl)-1H-pyrazole-4-carboxylate (33_Int-3)

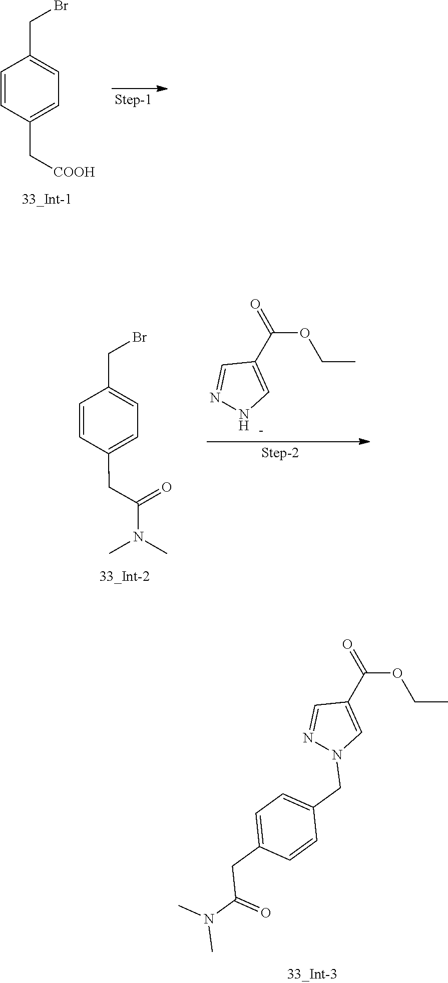

### Step-1. Synthesis of 2-(4-(bromomethyl)phenyl)-N-methylacetamide (33_Int-2)

A stirred solution of 2-(4-(bromomethyl)phenyl) acetic acid (33_Int-1)(1.0 g, 4.36 mmol, 1.0 eq) was prepared in toluene (10 mL, 10V). SOCl₂ (0.54 g, 4.53 mmol, 1.04 eq) and DMF (0.051 g, 0.69 mmol, 0.16 eq) were added at RT and stirred at 85° C. for 3 h. The RM was cooled to 0° C. followed by addition of Dimethylamine (2M in THF)(2 mL, 2V). The RM was brought to RT and stirred for 6 h. After completion of the reaction, the RM was quenched in a saturated NaHCO₃ solution and extracted with ethyl acetate. The combined organic fractions were dried over Na₂SO₄; concentrated to give compound (33_Int-2). MS(ES): 256.6 m/z [M]+258.1.m/z [M+2]+, LCMS purity: 98.96%, ¹H NMR (400 MHz, DMSO-d6) δ 2.826 (s, 3H), 2.997 (s, 3H), 3.694 (s, 2H), 4.706 (s, 3H), 7.210 (d, J=8 Hz, 2H), 7.385 (d, J=8 Hz, 2H).

### Step-2. Synthesis of ethyl 1-(4-(2-(dimethylamino)-2-oxoethyl)benzyl)-1H-pyrazole-4-carboxylate (33_Int-3)

A stirred solution of ethyl 1H-pyrazole-4-carboxylate (0.3 g, 2.14 mmol, 1.0 eq) was prepared in acetone (6 mL, 20V). 2-(4-(bromomethyl)phenyl)-N,N-dimethylacetamide (33_Int-2)(0.657 g, 2.56 mmol, 1.2 eq) and Cs₂CO₃ (1.67 g, 5.13 mmol, 2.4 eq) were added at RT. The RM was then heated to 65° C. and stirred for 16 h. After completion of the reaction, the RM was cooled to RT. The RM was filtered to remove Cs₂CO₃ and washed with ethyl acetate. The solid residue was discarded and filtrate was concentrated and then purified by column chromatography (25-30% ethyl acetate in hexanes) to give compound (33_Int-3) MS(ES): 316.3 m/z [M+1]+, LCMS purity: 100%, ¹H NMR (400 MHz, DMSO-d6) δ 1.252 (t, J=2.8 Hz, 3H), 2.802 (s, 3H), 2.974 (s, 3H), 3.396-3.295 (m, 2H), 4.188 (s, 2H), 5.324 (s, 2H), 7.189 (s, 4H), 7.852 (s, 1H), 8.466 (s, 1H).

### Preparation of Final Compounds

### Example 1: Preparation of N-(4-carbamimidoylbenzyl)-1-(4-(2-cyanopropan-2-yl)benzyl)-1H-pyrazole-4-carboxamide (7)

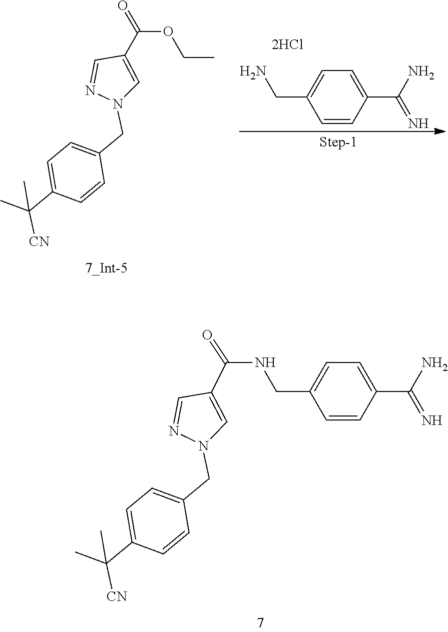 [SMILES: CCOC(=O)c1cnn(Cc2ccc(C(C)(C)C#N)cc2)c1 ; N=C(N)c1ccc(CN)cc1 ; CC(C)(C#N)c1ccc(Cn2cc(C(=O)NCc3ccc(C(=N)N)cc3)cn2)cc1]

A 3000 mL 4N RBF attached to mechanical stirrer and condenser was charged with ethyl 1-(4-(2-cyanopropan-2-yl)benzyl)-1H-pyrazole-4-carboxylate (7_Int5)(120 g, 403 mmol, 1.0 eq), 4-(aminomethyl)benzimidamide dihydrochloride (134.4 g, 605 mmol, 1.5 eq) and Toluene (1200 mL, 10V) at RT. DIPEA (130.14 g, 1008 mmol, 2.5 eq) was added to RM. The RM was cooled to 0° C. to 5° C. and stirred for 20 min. TMA (2M in toluene)(605.3 mL, 121 mmol, 3.0 eq) was added dropwise to RM. The RM was heated to 95° C. and stirred for 16 h. After completion of reaction, the reaction mixture was cooled to RT. The RM was slowly quenched with DM water (151 mL) and evaporated to obtain residue. filtrate was concentrated under reduced pressure to obtain crude. The crude was purified three times by column chromatography using 60-120 mesh size silica. Product was eluted at 10% MeOH in DCM to obtained solid, was dissolved in 4V Methanol, and stirred at 70° C. for 2 h. EtOAc was added at 60° C. until a turbid solution was observed. Solution was slowly cooled to RT and stirred for 16 h until a solid precipitate was obtained which was collected by filtration. Filtrate was evaporated and purified by column chromatography to give compound (7). MS(ES): 473.87 m/z [M+H]+, LCMS purity: 100%, HPLC purity: 95.07% ¹H NMR (400 MHz, DMSO-d6) δ 1.682 (s, 6H), 4.470 (s, 2H), 5.362 (s, 2H), 7.326 (d, J=8.4 Hz, 2H), 7.515-7.419 (m, 4H), 7.772 (d, J=8.4 Hz, 2H), 7.939 (s, 1H), 8.316 (s, 1H), 8.865-8.835 (m, 1H), 9.123 (s, 2H), 9.303 (s, 2H).

### Example 2: N-(4-carbamimidoylbenzyl)-1-(4-(2-cyanoethyl)benzyl)-1H-pyrazole-4-carboxamide (3)

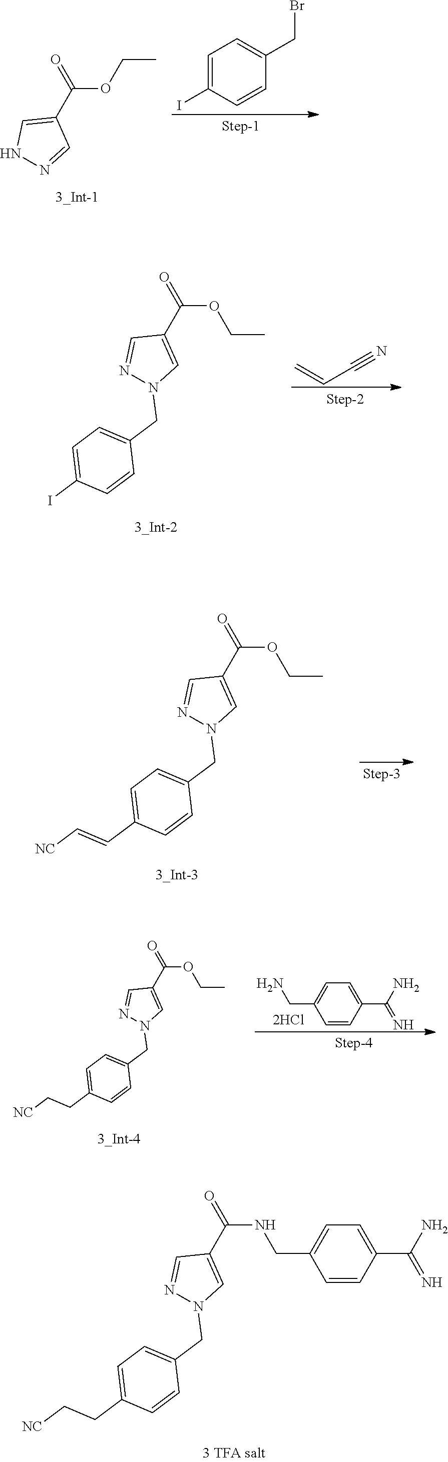

### Step-1. Synthesis of ethyl 1-(4-iodobenzyl)-1H-pyrazole-4-carboxylate (3_Int-2)

A stirred solution of 1H-pyrazole-4-carboxylate (0.5 g, 3.56 mmol, 1.0 eq) in DMF (5 mL, 10V) was prepared and NaH (60% in mineral oil)(0.213-g, 5.35 mmol, 1.5 eq) was added at 0° C. The RM was stirred at 0° C. for 30 minutes. 1-(bromomethyl)-4-iodobenzene (1.05 g, 3.56 mmol, 1.0 eq) was added at 0° C. and the RM was brought to RT and stirred for 2 h. After completion of the reaction, the RM was quenched in water and extracted by ethyl acetate. The combined organic fractions were dried over Na₂SO₄; concentrated and then purified by flash column chromatography (12-15% ethyl acetate in hexanes) to give compound (3_Int-2) MS(ES): 357.1.m/z [M+H]+, LCMS purity: 100%, 1H 400 MHz, DMSO-d6: δ 1.25 (t, J=7.20 Hz, 3H), 4.20 (d, J=7.20 Hz, 2H), 5.33 (s, 2H), 7.066 (d, J=7.60 Hz, 2H), 7.715 (d, J=8.00 Hz, 2H), 7.87 (s, 1H), 8.47 (s, 1H).

### Step-2. Synthesis of ethyl (E)-1-(4-(2-cyanovinyl)benzyl)-1H-pyrazole-4-carboxylate (3_Int-3)

A stirred solution of ethyl 1-(4-iodobenzyl)-1H-pyrazole-4-carboxylate (3_Int-2)(0.55 g, 1.54 mmol, 1.0 eq) and 1,4-Dioxane (11 mL, 20V) was prepared and Acrylonitrile (0.08 g, 1.54 mmol, 1 eq) and TEA (0.390 g, 3.86 mmol, 2.5 eq) were added at RT. N2 was purged in RM for 10 min at RT. Palladium diacetate (0.034 g, 0.15 momol, 0.1 eq) and JohnPhos (0.092 g, 0.30 momol, 0.2 eq) were added and the RM was heated to 110° C. and stirred for 3 h. After completion of the reaction, the RM was quenched in water and extracted by ethyl acetate. The combined organic fractions were dried over Na₂SO₄; concentrated and then purified by flash column chromatography (22-25% ethyl acetate in hexanes) to give compound (3_Int-3) MS(ES): 282.30.m/z [M+H]+, LCMS purity: 99.41%, 1H 400 MHz, DMSO-d6: δ 1.29 (d, J=5.6 Hz, 3H), 2.231 (d, J=5.6 Hz, 2H), 5.441 (d, J=7.2 Hz, 2H), 5.92 (d, J=12 Hz, 1H), 7.444-7.328 (m, Hz, 3H), 7.799 (d, J=5.6 Hz, 2H), 7.907 (s, Hz, 1H), 8.533 (s, 1H).

### Step-3. Synthesis of ethyl 1-(4-(2-cyanoethyl)benzyl)-1H-pyrazole-4-carboxylate (3_Int-4)

A stirred solution of (E)-1-(4-(2-cyanovinyl)benzyl)-1H-pyrazole-4-carboxylate (3_Int-3)(0.32 g, 1.13 mmol, 1.0 eq) in THF (6 mL, 20V) was prepared and 5% Pd/C was added at RT. RM was stirred under H₂ atmosphere at RT for 8 h. The RM was filtered by celite bed washed with ethyl acetate. Filtrate was concentrated and then purified by column chromatography (20-22% EtOAc in Hexanes) to give compound (3_Int-4). MS(ES): 284.30.m/z [M+H]+, LCMS purity: 65.00%, 1H 400 MHz, DMSO-d6: δ 1.32 (t, J=6.8 Hz, 3H), 2.81 (d, J=6.00 Hz, 2H), 2.86 (d, J=6.00 Hz, 2H), 4.22 (d, J=6.80 Hz, 2H), 5.36 (s, 2H), 7.27 (d, J=7.60 Hz, 2H), 7.66 (d, J=8.80 Hz, 2H), 7.87 (s, 1H), 8.50 (s, 1H).

### Step-4. Synthesis of N-(4-carbamimidoylbenzyl)-1-(4-(2-cyanoethyl)benzyl)-1H-pyrazole-4-carboxamide (3)

A stirred solution of ethyl 1-(4-(2-cyanoethyl)benzyl)-1H-pyrazole-4-carboxylate (3_Int-4)(0.2 g, 7.05 mmol, 1.0 eq) in toluene (2 mL, 10V) was prepared and 4-(aminomethyl)benzimidamide dihydrochloride (0.15 g, 10.58 mmol, 1.5 eq) and DIPEA (0.23 g, 17.64 mmol, 2.5 eq) were added at RT. After cooling to 0° C., TMA (2.0M in toluene)(1 mL, 2.11 mmol, 3.0 eq) was added and the RM was heated to 100° C. and stirred for 16 h. After completion of the reaction, the RM was quenched by 1V of water and evaporated. Residue was washed with 20% methanol in dichloromethane and filtered. Filtrate was concentrated and purified by Prep HPLC ((A)_{0.1}% TFA in water (B)₁₀₀% MeCN)) to give compound (3). MS(ES): 387.29.m/z [M+H]+, LCMS purity: 100%, HPLC purity: 100% 1H 400 MHz, DMSO-d6: δ 2.80 (d, J=6.00 Hz, 2H), 2.84 (d, J=6.00 Hz, 2H), 4.48 (d, J=5.60 Hz, 2H), 5.33 (s, 2H), 7.25 (q, J=27.20 Hz, 3H), 7.49 (d, J=8.00 Hz, 1H), 7.74 (d, J=8.00 Hz, 1H), 0.00 (s, 1H), 8.27 (s, 1H), 8.78 (d, J=6.00 Hz, 1H), 8.95 (s, 2H), 9.25 (s, 2H).

### Example 3: N-(4-carbamimidoylbenzyl)-1-(4-(2-cyanoethyl)benzyl)-1H-imidazole-4-carboxamide (4)

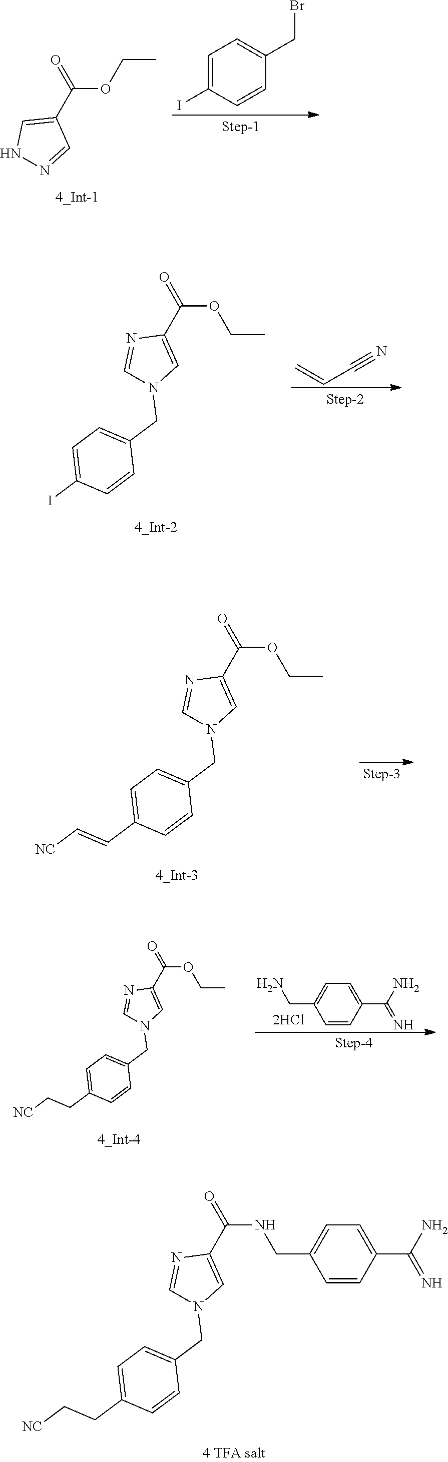

### Step-1. Synthesis of ethyl 1-(4-iodobenzyl)-1H-imidazole-4-carboxylate (4_Int-2)

A stirred solution of ethyl 1H-imidazole-4-carboxylate (0.5 g, 3.56 mmol, 1.0 eq) in DMF (5 mL, 10V) was prepared and NaHMDS (1M in THF)(3.5 mL, 3.56 mmol, 1.0 eq) was added at 0° C. and stirred for 30 min. 1-(bromomethyl)-4-iodobenzene (0.105 g, 3.56 mmol, 1.0 eq) was added to the RM at 0° C., brought to RT and stirred for 2 h. After completion of the reaction, the RM was quenched in water and extracted by ethyl acetate. The combined organic fractions were dried over Na₂SO₄; concentrated and then purified by flash column chromatography (35-38% ethyl acetate in hexanes) to give compound (4_Int-2). MS(ES): 357.20.m/z [M+H]+, LCMS purity: 99.05%, 1H 400 MHz, DMSO-d6: δ 1.24 (t, J=7.2 Hz, 3H), 4.19 (q, J=7.2 Hz, 2H), 5.20 (s, 2H), 7.12 (d, J=8.00 Hz, 2H), 7.74 (d, J=8.00 Hz, 2H), 7.88 (s, 1H), 7.94 (s, 1H).

### Step-2. Synthesis of ethyl (E)-1-(4-(2-cyanovinyl)benzyl)-1H-pyrazole-4-carboxylate (4_Int-3)

A stirred solution of ethyl 1-(4-iodobenzyl)-1H-imidazole-4-carboxylate (4_Int-2)(0.7 g, 1.96 mmol, 1.0 eq) and 1,4-Dioxane (14 mL, 20V) was prepared and Acrylonitrile (0.104 g, 1.96 mmol, 1 eq) and TEA (0.497 g, 4.91 mmol, 2.5 eq) were added at RT. N2 was purged in RM for 10 min at RT. Palladium diacetate (0.044 g, 0.19 mmol, 0.1 eq) and JohnPhos (0.117 g, 0.39 momol, 0.2 eq) were added to RM. RM was heated to 110° C. and stirred for 3 h. RM was quenched in water and extracted by ethyl acetate. The combined organic fractions were dried over Na₂SO₄; concentrated and then purified by flash column chromatography (22-25% ethyl acetate in hexanes) to give compound (3_Int-3). MS(ES): 282.28.m/z [M+H]+, LCMS purity: 89.04%, 1H 400 MHz, DMSO-d6: δ 1.23 (t, J=7.2 Hz, 3H), 4.21 (q, J=5.60 Hz, 2H), 5.22 (s, 2H), 6.49 (d, J=16.80 Hz, 1H), 7.41 (t, J=6.8 Hz, 3H), 7.66 (d, J=6 Hz, 1H), 7.82 (d, J=7.60 Hz, 2H), 7.99 (s, 1H).

### Step-3. Synthesis of ethyl 1-(4-(2-cyanoethyl)benzyl)-1H-imidazole-4-carboxylate (4_Int-4)

A stirred solution of ethyl (E)-1-(4-(2-cyanovinyl)benzyl)-1H-imidazole-4-carboxylate (4_Int-3)(0.4 g, 1.42 mmol, 1.0 eq) in THF (5 mL, 12V) and Ethanol (5 mL, 12V) was prepared and 5% Pd/C were added at RT. RM was stirred under hydrogen atmosphere at RT for 16 h. After completion of the reaction, the RM was filtered by celite bed washed with ethyl acetate. Filtrate was concentrated and then purified by column chromatography (38-40% EtOAc in Hexanes) to give compound (3_Int-4). MS(ES): 284.22.m/z [M+H]+, LCMS purity: 69.16.

Step-4. N-(4-carbamimidoylbenzyl)-1-(4-(2-cyanoethyl)benzyl)-1H-pyrazole-4-carboxamide (4) was prepared from ethyl 1-(4-(2-cyanoethyl)benzyl)-1H-imidazole-4-carboxylate (4_Int-4) and 4-(aminomethyl)benzimidamide dihydrochloride in a similar fashion to that described in Example-2 (4). MS(ES): 387.30.m/z [M+H]+, LCMS purity: 100%, HPLC purity: 100% 1H 400 MHz, DMSO-d6: δ 2.80 (d, J=6.40 Hz, 2H), 2.85 (d, J=6.00 Hz, 2H), 4.47 (d, J=5.60 Hz, 2H), 5.23 (s, 2H), 7.30 (s, 3H), 7.46 (d, J=28.00 Hz, 2H), 7.74 (t, J=8 Hz, 3H), 8.00 (s, 1H), 8.74 (s, 1H), 8.95 (s, 2H), 9.24 (s, 2H).

### Example 4: Preparation of N-(4-carbamimidoyl-3-fluorobenzyl)-1-(4-(2-cyanopropan-2-yl)benzyl)-1H-pyrazole-4-carboxamide (43)

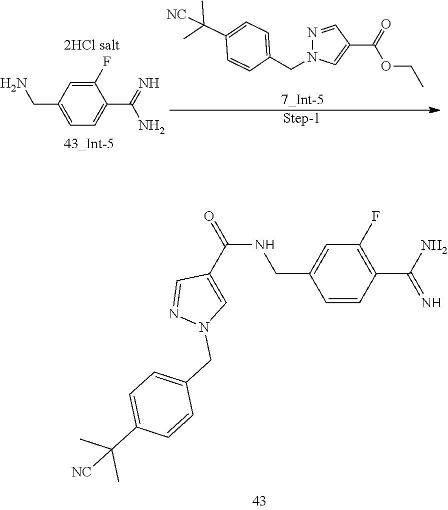 [SMILES: N=C(N)c1ccc(CN)cc1F ; CCOC(=O)c1cnn(Cc2ccc(C(C)(C)C#N)cc2)c1 ; CC(C)(C#N)c1ccc(Cn2cc(C(=O)NCc3ccc(C(=N)N)c(F)c3)cn2)cc1]

N-(4-carbamimidoyl-3-fluorobenzyl)-1-(4-(2-cyanopropan-2-yl)benzyl)-1H-pyrazole-4-carboxamide (301) was prepared from ethyl 1-(4-(2-cyanopropan-2-yl)benzyl)-1H-pyrazole-4-carboxylate (7_Int-5) and 4-(aminomethyl)-2-fluorobenzimidamide dihydrochloride (43_Int-5) in a similar fashion to that described in Example-2 (3). MS(ES): 419.20.m/z [M+H]+, LCMS purity: 100%, HPLC purity: 10% ¹H NMR (400 MHz, DMSO-d6) δ 1.667 (s, 6H), 2.504 (s, 2H), 4.771 (d, J=4.2 Hz, 2H), 5.368 (s, 2H), 7.343-7.324 (m, 4H), 7.511 (d, J=8 Hz, 2H), 7.625 (t, J=8 Hz, 1H), 7.919 (s, 1H), 8.295 (s, 1H), 8.795 (t, J=6 Hz, 1H), 9.184 (s, 2H), 9.369 (s, 2H).

### Example 5: N-(4-carbamimidoylbenzyl)-1-(4-(cyanomethyl) phenethyl)-1H-pyrazole-4-carboxamide (12)

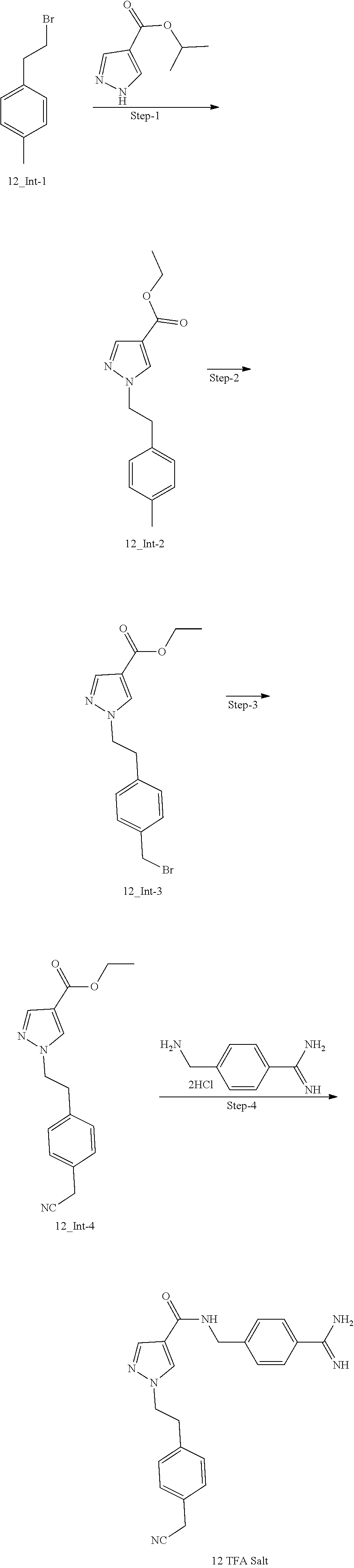

### Step-1. Synthesis of ethyl 1-(4-methylphenethyl)-1H-pyrazole-4-carboxylate (12_Int-2)

A stirred solution of ethyl 1H-pyrazole-4-carboxylate (2 g, 14.28 mmol, 1.0 eq) in acetone (20 mL, 10V) was prepared and 1-(2-bromoethyl)-4-methylbenzene (304 g, 17.14 mmol, 1.2 eq) and Cs₂CO₃ (11.14 g, 34.28 mmol, 2.4 eq) at RT. The RM was heated to 60-65° C., stirred for 16 h. and cooled to RT. The RM was filtered, washed with EtOAc, and purified by column chromatography (12-15% ethyl acetate in hexanes) to give compound (12_Int-2) MS(ES): 259.23.m/z [M+H]+.

### Step-2. Synthesis of ethyl 1-(4-(bromomethyl) phenethyl)-1H-pyrazole-4-carboxylate (12_Int-3)

A stirred solution of ethyl 1-(4-methylphenethyl)-1H-pyrazole-4-carboxylate 12_Int-2). (1.7 g, 6.58 mmol, 1.0 eq) in Carbon tetrachloride (34 mL, 20V) was prepared and Benzoyl peroxide (0.159 g, 0.65 mmol, 0.1 eq) and N-Bromosuccinimide (1.40 g, 7.89 mmol, 1.2 eq) were added at RT. The RM was stirred for 4 h at RT, quenched in water, and extracted by DCM. The combined organic fractions were dried over Na₂SO₄; concentrated and then purified by flash column chromatography (10-12% ethyl acetate in hexanes) to give compound (12_Int-3). MS(ES): 337.4.m/z [M]+339.4 [M+2]+, LCMS purity: 75.91%, ¹H NMR (400 MHz, DMSO-d6) δ 1.239 (t, J=7.2 Hz, 3H), 3.111 (t, J=7.2 Hz, 2H), 4.179 (q, J=7.2 Hz, 2H), 4.385 (t, J=7.2 Hz, 2H), 4.664 (s, 2H), 7.148-7.127 (m, 2H), 7.350-7.330 (m, 2H), 7.841 (s, 1H), 8.220 (s, 1H).

### Step-3. Synthesis of ethyl 1-(4-(cyanomethyl) phenethyl)-1H-pyrazole-4-carboxylate (12_Int-4)

A stirred solution of ethyl 1-(4-(bromomethyl) phenethyl)-1H-pyrazole-4-carboxylate (12_Int-3)(0.750 g, 2.22 mmol, 1.0 eq) in MeCN (6.mL, 20V) was prepared at RT. Tetrabutylammonium cyanide (2.38 g, 8.89 mmol, 4.0 eq) was added and the RM stirred for 16 h at RT. After completion of the reaction, the RM was quenched in water and extracted by ethyl acetate. The combined organic fractions were dried over Na₂SO₄; concentrated and then purified by flash column chromatography (7-10% ethyl acetate in hexanes) to give compound (12_Int-4). MS(ES): 284.28 m/z [M+H]+, LCMS purity: 93.34%, ¹H NMR (400 MHz, DMSO-d6) δ 1.239 (t, J=7.2 Hz, 3H), 3.112 (t, J=7.2 Hz, 2H), 3.975 (s, 2H), 4.188 (q, J=7.2 Hz, 2H), 4.383 (t, J=7.2 Hz, 2H), 7.164 (d, J=8 Hz, 2H), 7.238 (d, J=8 Hz, 2H), 7.839 (s, 1H), 8.216 (s, 1H).

### Step-4. Synthesis of N-(4-carbamimidoylbenzyl)-1-(4-(cyanomethyl) phenethyl)-1H-pyrazole-4-carboxamide (12)

The title compound was prepared from ethyl 1-(4-(cyanomethyl) phenethyl)-1H-pyrazole-4-carboxylate (12_Int-4) and 4-(aminomethyl)benzimidamide dihydrochloride in a similar fashion as described in Example 2. MS(ES): 387.37 m/z [M+H]+, LCMS purity: 100%, HPLC purity: 95.02% ¹H NMR (400 MHz, DMSO-d6) δ 3.110 (t, J=7.2 Hz, 2H), 3.979 (s, 2H), 4.367 (t, J=7.2 Hz, 2H), 4.771 (d, J=6 Hz, 2H), 7.182 (d, J=8 Hz, 2H), 7.244 (d, J=8 Hz, 2H), 7.485 (d, J=8 Hz, 2H), 7.751 (d, J=8 Hz, 2H), 7.900 (s, 1H), 8.086 (s, 1H), 8.720 (t, J=6 Hz, 1H), 8.974 (s, 2H), 9.248 (s, 2H).

### Example 6: Preparation of 2-(4-((4-((4-carbamimidoylbenzyl) carbamoyl)-1H-pyrazol-1-yl)methyl)phenyl) acetic acid (16)

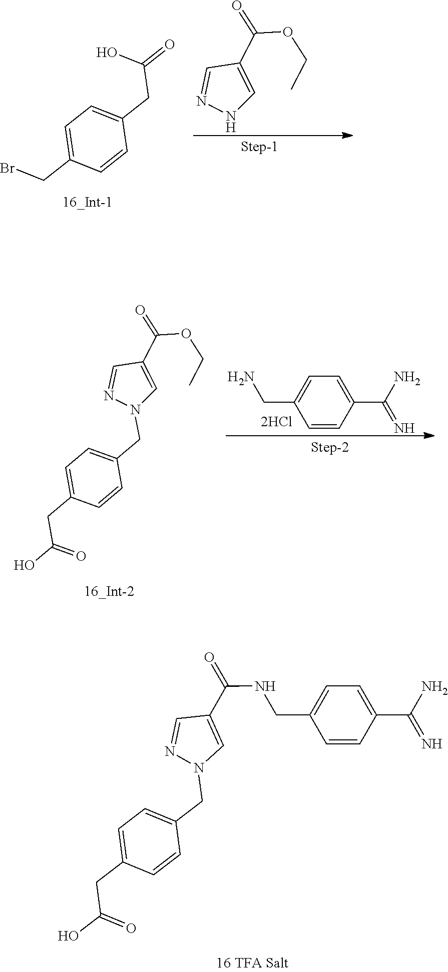

### Step-1. Synthesis of 2-(4-((4-(ethoxycarbonyl)-1H-pyrazol-1-yl)methyl)phenyl) acetic acid (16_Int-2)

A stirred solution of 1H-pyrazole-4-carboxylate (0.5 g, 3.56 mmol, 1.0 eq) in DMF (5 mL, 10V) was prepared and NaH was added (60% in mineral oil)(0.214 g, 5.35 mmol, 1.5 eq) at 0° C. The RM was stirred at 0° C. for 30 minutes. 2-(4-(bromomethyl)phenyl) acetic acid (0.980 g, 4.28 mmol, 1.2 eq) was added to RM at 0° C. and was stirred at RT for 16 h. After completion of the reaction, the RM was quenched in water and extracted by ethyl acetate. The combined organic fractions were dried over Na₂SO₄; concentrated and then purified by flash column chromatography (5-7% methanol in DCM) to give compound (16_Int-2) MS(ES): 289.3 m/z [M+H]+, LCMS purity: 47.20%, 1H NMR (400 MHz, DMSO-d6) δ 1.252 (t, J=7.2 Hz, 3H), 3.542 (s, 2H), 4.201 (q, J=7.2 Hz, 2H), 5.335 (s, 2H), 7.222 (s, 4H), 7.853 (s, 1H), 8.451 (s, 1H), 12.321 (s, 1H).

Step-2. 2-(4-((4-((4-carbamimidoylbenzyl) carbamoyl)-1H-pyrazol-1-yl)methyl)phenyl) acetic acid (16) was prepared from 2-(4-((4-(ethoxycarbonyl)-1H-pyrazol-1-yl)methyl)phenyl) acetic acid (16_Int-2), and 4-(aminomethyl)benzimidamide dihydrochloride in a similar fashion to that described in Example-2 (16). MS(ES): 392.50 m/z [M+H]+, LCMS purity: 100%, HPLC purity: 99.29% ¹H NMR (400 MHz, DMSO-d6) δ 1.841 (s, 2H), 3.503 (s, 2H), 4.785 (s, 2H), 7.073-7.051 (m, 4H), 7.297 (d, J=8 Hz, 2H), 7.504 (d, J=8 Hz, 2H), 7.794 (s, 1H), 8.065 (s, 1H).

### Example 7: Preparation of 1-(4-(2-amino-2-oxoethyl)benzyl)-N-(4-carbamimidoylbenzyl)-1H-pyrazole-4-carboxamide (18)

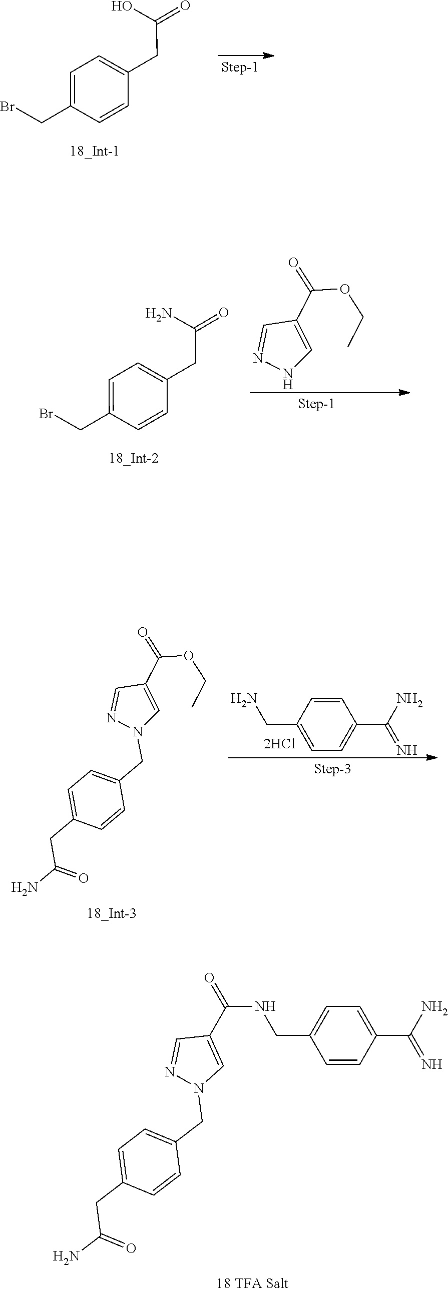

### Step-1. Synthesis of 2-(4-(bromomethyl)phenyl) acetamide (18_Int-2)

A stirred solution 2-(4-(bromomethyl)phenyl) acetic acid (0.5 g, 2.18 mmol, 1.0 eq) in toluene (5 mL, 10V) was prepared and SOCl₂ (0.270 g, 2.26 mmol, 1.04 eq) and DMF (0.025 g, 0.34 mmol, 0.16 eq) were added at RT. The RM was heated 80-85° C. for 3 h followed by an ammonia gas purge at 0° C. for 3 h. The RM was filtered and solids were washed with EtOAc to give compound (18_Int-2). MS(ES): 228.30 m/z [M]+, 230.12 m/z [M+2]+.

### Step-2. Synthesis of ethyl 1-(4-(2-amino-2-oxoethyl)benzyl)-1H-pyrazole-4-carboxylate (18_Int-3)

A stirred solution of 1H-pyrazole-4-carboxylate (18_Int-2)(0.5 g, 3.56 mmol, 1.0 eq) in DMF (5 mL, 10V) was prepared and NaH (60% in mineral oil)(0.214 g, 5.35 mmol, 1.5 eq) was added at 0° C. The RM was stirred at 0° C. for 30 minutes. 2-(4-(bromomethyl)phenyl) acetamide (0.976 g, 4.28 mmol, 1.2 eq) was added to the RM at 0° C. and stirred at RT for 16 h. After completion of the reaction, the RM was quenched in water and extracted by ethyl acetate. The combined organic fractions were dried over Na₂SO₄; concentrated and then purified by flash column chromatography (40-42% ethyl acetate in hexanes) to give compound (18_Int-3). MS(ES): 288.23.m/z [M+H]+, LCMS purity: 97.01%, ¹H NMR (400 MHz, DMSO-d6) δ 1.244 (t, J=7.2 Hz, 3H), 3.231 (s, 3H), 4.201 (q, J=7.2 Hz, 2H), 4.210 (s, 2H), 5.320 (s, 2H), 7.210 (s, 4H), 7.917-7.848 (m, 2H), 8.436 (s, 1H).

### Step-3. Synthesis of 1-(4-(2-amino-2-oxoethyl)benzyl)-N-(4-carbamimidoylbenzyl)-1H-pyrazole-4-carboxamide (18)

The title compound was prepared from ethyl 1-(4-(2-amino-2-oxoethyl)benzyl)-1H-pyrazole-4-carboxylate (18_Int-3) and 4-(aminomethyl)benzimidamide dihydrochloride in a similar fashion to that described in Example 2. MS(ES): 391.32.m/z [M+H]+, LCMS purity: 99.23%, HPLC purity: 98.73% ¹H NMR (400 MHz, DMSO-d6) δ 3.321 (d, J=3.2 Hz, 2H), 4.479 (d, J=6 Hz, 2H), 5.313 (s, 2H), 6.489 (s, 1H), 7.247-7.193 (m, 4H), 7.505-7.449 (m, 3H), 7.748 (d, J=8 Hz, 1H), 7.902 (s, 1H), 8.253 (s, 1H), 8.752 (t, J=6 Hz, 1H), 8.967 (s, 2H), 9.244 (s, 2H).

### Example 8: Preparation of N-(4-carbamimidoylbenzyl)-1-(4-(2-(methylamino)-2-oxoethyl)benzyl)-1H-pyrazole-4-carboxamide (19)

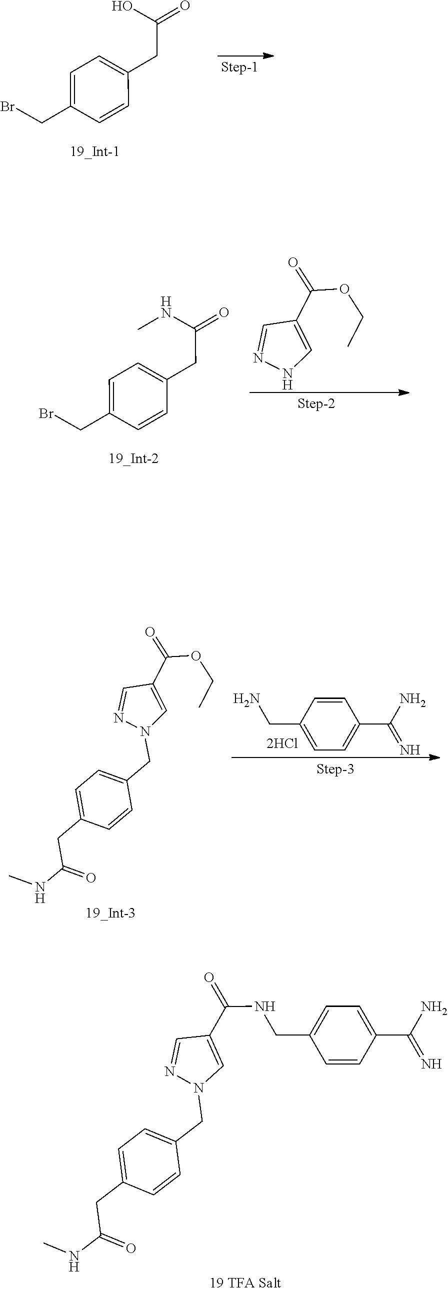

### Step-1. Synthesis of 2-(4-(bromomethyl)phenyl)-N-methylacetamide (19_Int-2)

A stirred solution of 2-(4-(bromomethyl)phenyl) acetic acid (1.0 g, 4.36 mmol, 1.0 eq) in Toluene (5 mL, 10V) was prepared at RT. SOCl₂ (0.54 g, 4.53 mmol, 1.04 eq) and DMF (0.051 g, 0.69 mmol, 0.16 eq) were added at RT and then heated to 80-85° C. for 3 h. That solution was cooled to 0° C. and methylamine (2 mL) was added to the RM and stirred at RT for 6 h. The RM was filtered and solids were washed with EtOAc to give compound (19_Int-2). MS(ES): 242.1.m/z [M]+ 244.1.m/z [M+2]+, LCMS purity: 96.6%, ¹H NMR (400 MHz, DMSO-d6) δ 3.389 (s, 2H), 4.726 (s, 2H), 7.245 (d, J=8 Hz, 2H), 7.344 (d, J=8 Hz, 2H), 8.019 (s, 1H).

### Step-2. Synthesis of ethyl 1-(4-(2-(methylamino)-2-oxoethyl)benzyl)-1H-pyrazole-4-carboxylate (19_Int-3)

A stirred solution of ethyl 1H-pyrazole-4-carboxylate (0.5 g, 3.56 mmol, 1.0 eq) in acetone (10 mL, 20V) was prepared and 2-(4-(bromomethyl)phenyl)-N-methylacetamide (19_Int-2)(1.03 g, 4.28 mmol, 1.2 eq) and s₂CO₃ (2.78 g, 8.56 mmol, 2.4 eq) were added at RT. The RM was heated to 60-65° C. and stirred for 16 h. After completion of the reaction, the RM was cooled to RT. The RM was filtered and washed with EtOAc. Solid residue was discarded and filtrate was concentrated and purified by column chromatography (12-15% ethyl acetate in hexanes) to give compound (19_Int-3) MS(ES): 302.40.m/z [M+H]+, LCMS purity: 86.48%, ¹H NMR (400 MHz, DMSO-d6) δ 1.658 (s, 6H), 4.556 (s, 2H), 5.366 (s, 2H), 7.331 (d, J=6 Hz, 2H), 7.505 (d, J=8 Hz, 4H), 7.983-7.934 (m, 2H), 8.314 (s, 1H), 8.595 (s, 1H), 8.813 (s, 1H).

Step-3. N-(4-carbamimidoylbenzyl)-1-(4-(2-(methylamino)-2-oxoethyl)benzyl)-1H-pyrazole-4-carboxamide (19) was prepared from ethyl 1-(4-(2-(methylamino)-2-oxoethyl)benzyl)-1H-pyrazole-4-carboxylate (19_Int-3) and 4-(aminomethyl)benzimidamide dihydrochloride in a similar fashion to that described in Example 2. MS(ES): 405.55.m/z [M+H]+, LCMS purity: 100%, HPLC purity: 100% ¹H NMR (400 MHz, DMSO-d6) δ 2.545 (d, J=4.8 Hz, 3H), 3.361 (s, 2H), 4.476 (d, J=6 Hz, 2H), 5.306 (s, 2H), 7.134-7.186 (m, 4H), 7.492 (d, J=8 Hz, 2H), 7.743 (d, J=8 Hz, 2H), 7.916-7.896 (m, 2H), 8.246 (s, 1H), 8.748 (t, J=6 Hz, 1H), 8.896 (s, 2H), 9.234 (s, 2H).

### Example 9: Preparation of N-(4-carbamimidoylbenzyl)-1-(4-((3-cyano-1H-pyrrol-1-yl)methyl)benzyl)-1H-pyrazole-4-carboxamide (26)

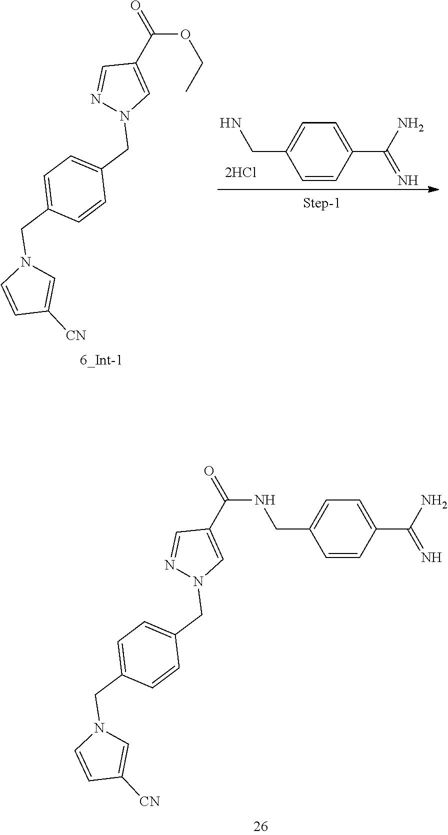 [SMILES: CCOC(=O)c1cnn(Cc2ccc(Cn3ccc(C#N)c3)cc2)c1 ; N=C(N)c1ccc(CN)cc1 ; N#Cc1ccn(Cc2ccc(Cn3cc(C(=O)NCc4ccc(C(=N)N)cc4)cn3)cc2)c1]

N-(4-carbamimidoylbenzyl)-1-(4-((3-cyano-1H-pyrrol-1-yl)methyl)benzyl)-1H-pyrazole-4-carboxamide (26) was prepared from ethyl 1-(4-((3-cyano-1H-pyrrol-1-yl)methyl)benzyl)-1H-pyrazole-4-carboxylate (6_Int-1) and 4-(aminomethyl)benzimidamide dihydrochloride in a similar fashion to that described in Example. MS(ES): 43841.m/z [M+H]+, LCMS purity: 100%, HPLC purity: 100% ¹H NMR (400 MHz, DMSO-d6) δ 4.482 (d, J=6 Hz, 2H), 5.156 (s, 2H), 5.335 (s, 2H), 6.467 (s, 2H), 7.001 (s, 1H), 7.254-7.250 (m, 4H), 7.496 (d, J=8 Hz, 2H), 7.711 (s, 1H), 7.754 (d, J=8 Hz, 2H), 7.909 (s, 1H), 8.270 (s, 1H), 8.772 (t, J=6 Hz, 1H), 9.012 (s, 2H), 9.258 (s, 2H).

### Example 10: Preparation of N-(4-carbamimidoylbenzyl)-1-(4-(1-cyanoethyl)benzyl)-1H-pyrazole-4-carboxamide (5)

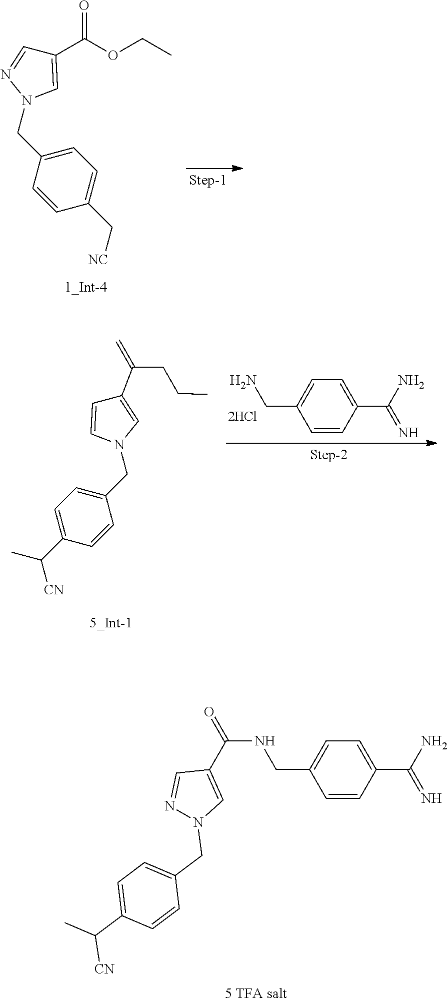

### Step-1. Synthesis of ethyl 1-(4-(1-cyanoethyl)benzyl)-1H-pyrazole-4-carboxylate (5_Int-1)

A stirred solution of ethyl 1-(4-(cyanomethyl)benzyl)-1H-pyrazole-4-carboxylate (1_int-4)(0.3 g, 1.11 mmol, 1.0 eq) in THF (6 mL, 20V), was prepared and LiHMDS was added (1M in hexane)(1.23 mL, 1.22 mmol, 1.1 eq) at −70° C. The RM was stirred at −70° C. for 30 min. Methyl iodide (0.2 g, 1.44 mmol, 1.3 eq) was added to the RM at −70° C. The RM was warmed to RT and stirred for 16 h. After completion of the reaction, the RM was quenched in water and extracted by ethyl acetate. The combined organic fractions were dried over Na₂SO₄; concentrated and then purified by flash column chromatography (12-15% ethyl acetate in hexanes) to give compound (5_Int-1). MS(ES): 284.28 m/z [M+H]+, LCMS purity: 46.39%.

Step-2. N-(4-carbamimidoylbenzyl)-1-(4-(1-cyanoethyl)benzyl)-1H-pyrazole-4-carboxamide (45) was prepared from ethyl 1-(4-(1-cyanoethyl)benzyl)-1H-pyrazole-4-carboxylate (5-Int-1) in a similar fashion to that described in Example-2. MS(ES): 387.33.m/z [M+H]+, LCMS purity: 94.00%.

### Example 11: Preparation of N-(4-carbamimidoyl-2-fluorobenzyl)-1-(4-(2-cyanopropan-2-yl)benzyl)-1H-pyrazole-4-carboxamide (45)

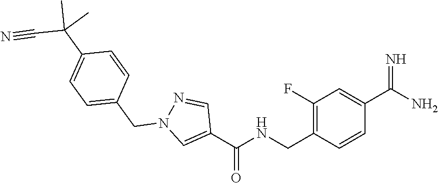 [SMILES: CC(C)(C#N)c1ccc(Cn2cc(C(=O)NCc3ccc(C(=N)N)cc3F)cn2)cc1]

N-(4-carbamimidoyl-2-fluorobenzyl)-1-(4-(2-cyanopropan-2-yl)benzyl)-1H-pyrazole-4-carboxamide (45) was prepared from ethyl 1-(4-(2-cyanopropan-2-yl)benzyl)-1H-pyrazole-4-carboxylate (4-Int-5) and 4-(aminomethyl)-3-fluorobenzimidamide dihydrochloride (45_Int-5) in a similar fashion to that described in Example-2. MS(ES): 419.4.m/z [M+H]+, LCMS purity: 100%, HPLC purity: 100% ¹H NMR (400 MHz, DMSO-d6) δ 1.664 (s, 6H), 4.499 (d, J=6.4 Hz, 2H), 5.366 (s, 2H), 7.331 (d, J=8 Hz, 2H), 7.554-7.500 (m, 3H), 7.608 (d, J=8 Hz, 1H), 7.771 (d, J=8 Hz, 1H), 7.925 (s, 1H), 8.303 (s, 1H), 8.776 (t. J=8.8 Hz, 1H), 9.115 (s, 2H), 9.334 (s, 2H), 19F NMR (400 MHz, DMSO-d6) δ −73-659 (1F), −116.871 (0.35F).

### Example 12: Preparation of N-(4-carbamimidoylbenzyl)-1-(4-(cyanomethyl)benzyl)-1H-pyrazole-4-carboxamide (1)

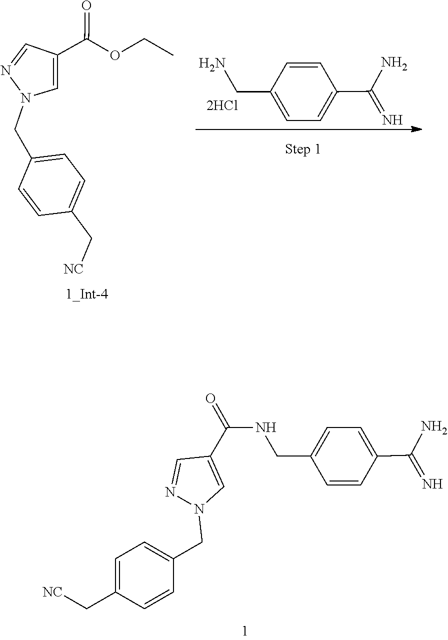 [SMILES: CCOC(=O)c1cnn(Cc2ccc(CC#N)cc2)c1 ; N=C(N)c1ccc(CN)cc1 ; N#CCc1ccc(Cn2cc(C(=O)NCc3ccc(C(=N)N)cc3)cn2)cc1]

A 3000 mL 4N RBF attached to mechanical stirrer and condenser was charged with ethyl 1-(4-(cyanomethyl)benzyl)-1H-pyrazole-4-carboxylate (1_Int4)(140 g, 517 mmol, 1.0 eq), 4-(aminomethyl)benzimidamide dihydrochloride (127 g, 571 mmol, 1.1 eq) and Toluene (1400 mL, 10V) at RT. DIPEA (266.5 mL, 1559 mmol, 3.0 eq) was added to RM. The RM was cooled to 0° C. to 5° C. and stirred for 20 min. TMA (2M in toluene)(649.8 mL, 1299 mmol, 2.5 eq) was added dropwise to RM. The RM was heated to 95° C. and stirred for 16 h. The RM was cooled to RT, slowly quenched with DM water (151 mL), and evaporated to obtain residue. The solid residue was washed with 50% methanol in DCM (3×1000 mL) and the filtrate was discarded. The solid residue washed again with 20% MeOH in DCM (2×2000 mL and then discarded. The filtrate was concentrated and purified by flash column chromatography (12-15% methanol in DCM) to give compound (1) MS(ES): 373.35 m/z [M+H]+, LCMS purity: 96.80%, HPLC purity: 95.11% ¹H NMR (400 MHz, DMSO-d6) δ 3.805 (s, 2H), 4.455 (s, 2H), 5.314 (s, 2H), 7.304-7.289 (m, 4H), 7.469 (d, J=8 Hz 2H), 7.710 (d, J=8 Hz, 2H), 7.897 (s, 1H), 8.246 (s, 1H).

### Example 13: Preparation of N-(4-carbamimidoylbenzyl)-1-(4-(hydroxymethyl)benzyl)-1H-pyrazole-4-carboxamide (2)

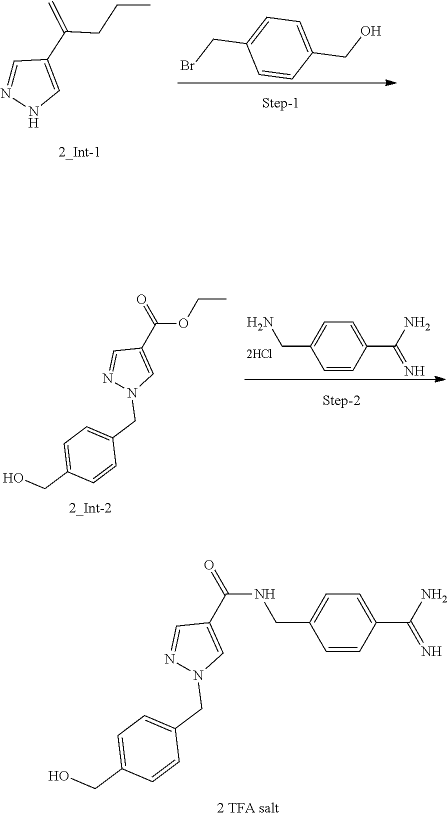

### Step-1. Synthesis of ethyl 1-(4-(hydroxymethyl)benzyl)-1H-pyrazole-4-carboxylate (2_Int-2)

A stirred solution of ethyl 1H-pyrazole-4-carboxylate (2_int-1) (0.5 g, 3.56 mmol, 1.0 eq) in acetone (5 mL, 10V) was prepared and (4-(bromomethyl)phenyl) methanol (0.86 g, 4.28 mmol, 1.2 eq) and Cs₂CO₃ (3.25 g, 9.98 mmol, 2.8 eq) were added at RT. The RM was heated to 60° C. and stirred for 3 h. After completion of the reaction, the RM was quenched in water and extracted by ethyl acetate. The combined organic fractions were dried over Na₂SO₄; concentrated and then purified by flash column chromatography (12-15% ethyl acetate in hexanes) to give compound (2_Int-2). MS(ES): 261.30 m/z [M+H]+, LCMS purity: 97.45%, ¹H NMR (400 MHz, DMSO-d6) δ 1.255 (s, 3H), 4.203 (d, J=6.4 Hz, 2H), 4.469 (s, 2H), 5.187 (s, 1H), 5.340 (s, 2H), 7.269 (m, 4H), 7.860 (s, 1H), 8.444 (s, 1H).

Step-2. N-(4-carbamimidoylbenzyl)-1-(4-(hydroxymethyl)benzyl)-1H-pyrazole-4-carboxamide (2) was prepared from ethyl 1-(4-(hydroxymethyl)benzyl)-1H-pyrazole-4-carboxylate (2_Int-2) and 4-(aminomethyl)benzimidamide dihydrochloride in a similar fashion as that described in Example-2. MS(ES): 364.28 m/z [M+H]+, LCMS purity: 100%, HPLC purity: 95.74% ¹H NMR (400 MHz, DMSO-d6) δ 4.469 (s, 1H), 5.199 (s, 1H), 5.321 (s, 2H), 7.282-7.223 (m, 4H), 7.485 (d, J=6.4 Hz, 2H), 7.741 (d, J=6.4 Hz, 2H), 7.904 (s, 1H), 8.251 (s, 1H), 8.776 (s, 1H), 8.995 (s, 2H), 9.250 (s, 2H), 19F NMR (400 MHz, DMSO-d6) δ −73.575 (1F).

### Example 14: Preparation of N-(4-carbamimidoylbenzyl)-1-(4-(2-cyanopropan-2-yl)benzyl)-1H-imidazole-4-carboxamide (8)

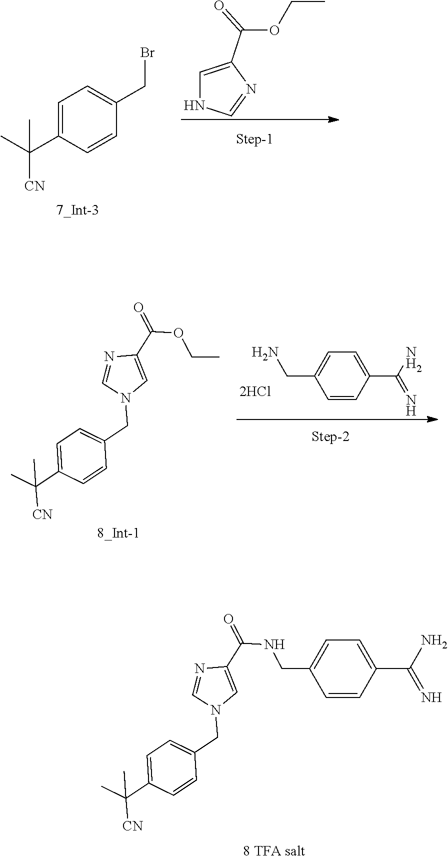

### Step-1. Synthesis of ethyl 1-(4-(2-cyanopropan-2-yl)benzyl)-1H-imidazole-4-carboxylate (8_Int-1)

A stirred solution of ethyl 1H-imidazole-4-carboxylate (0.45 g, 3.21 mmol, 1.0 eq) in DMF (4.5 mL, 10V) was prepared and LiHMDS (1M in THF)(3.2 mL, 3.21 mmol, 1.0 eq) was added at −78° C. and stirred for 30 minutes. 2-(4-(bromomethyl)phenyl)-2-methylpropanenitrile (7_int-3)(0.91 g, 3.85 mmol, 1.2 eq) was added to RM at −78° C. RM was cooled to RT and stirred at RT. After completion of the reaction, the RM was quenched in water and extracted by ethyl acetate. The combined organic fractions were dried over Na₂SO₄; concentrated and then purified by flash column chromatography (1.2-1.5% methanol in DCM) to give compound (8_Int-1). MS(ES): 297.85 m/z [M+H]+, LCMS purity: 86.41%, ¹H NMR (400 MHz, DMSO-d6) δ 1.246 (t, J=5.2 Hz, 3H), 1.649 (s, 6H), 4.220-4.185 (m, 2H), 5.256 (s, 2H), 7.381 (d, J=8 Hz, 2H), 7.524 (d, J=8 Hz, 2H), 7.895 (d, J=2 Hz, 1H), 7.952 (d, J=2 Hz, 1H).

Step-2. N-(4-carbamimidoylbenzyl)-1-(4-(2-cyanopropan-2-yl)benzyl)-1H-imidazole-4-carboxamide (8) was prepared from ethyl 1-(4-(2-cyanopropan-2-yl)benzyl)-1H-imidazole-4-carboxylate (8_Int-1) and 4-(aminomethyl)benzimidamide dihydrochloride in a similar fashion as that described in Example-2. MS(ES): 401.25 m/z [M+H]+, LCMS purity: 100%, HPLC purity: 99.63% ¹H NMR (400 MHz, DMSO-d6) δ 1.662 (s, 6H), 4.772 (d, J=6 Hz, 2H), 5.257 (s, 2H), 7.395 (d, J=8 Hz, 2H), 7.536-7.473 (m, 4H), 7.759-7.717 (m, 2H), 8.717 (s, 1H), 8.873 (s, 2H), 9.231 (s, 2H).

### Example 15: Preparation of N-(4-carbamimidoylbenzyl)-1-(4-(cyanomethyl)benzyl)-1H-imidazole-4-carboxamide (9)

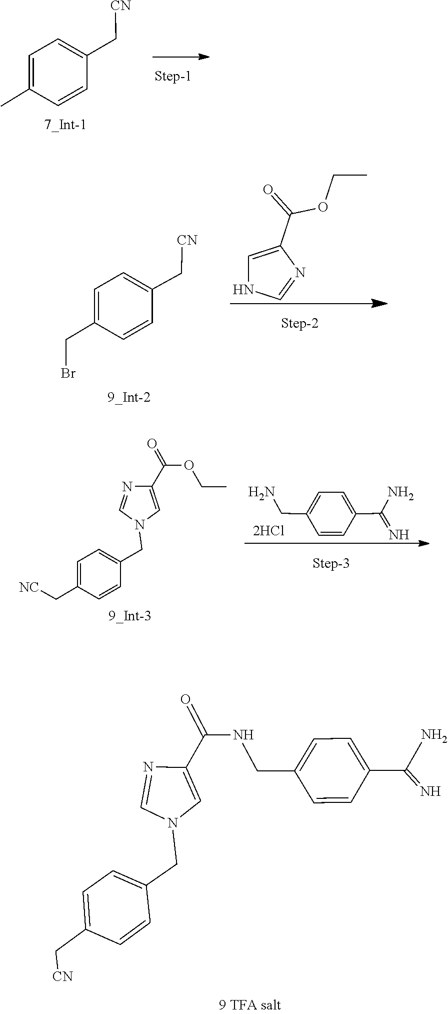

### Step-1. Synthesis of 2-(4-(bromomethyl)phenyl) acetonitrile (9_Int-2)

A stirred solution of 2-(p-tolyl) acetonitrile (2 g, 15.24 mmol, 1.0 eq) and carbon tetrachloride (40 mL, 20V) was prepared at RT. AIBN (0.12 g, 0.76 mmol, 0.1 eq) and N-Bromosuccinimide (2.24 g, 1.67 mmol, 1.1 eq) were added at RT and the RM was then heated to 90° C. and stirred for 16 h. After completion of the reaction, the RM was quenched in water and extracted by DCM. The combined organic fractions were dried over Na₂SO₄, concentrated and then purified by flash column chromatography (7-10% ethyl acetate in hexane) to give compound (9_Int-2). ¹H NMR (400 MHz, DMSO-d6) δ 4.092 (s, 2H), 4.705 (s, 2H), 7.335 (d, J=8 Hz, 2H), 7.469 (d, J=8 Hz, 2H).

### Step-2. Synthesis of ethyl 1-(4-(cyanomethyl)benzyl)-1H-imidazole-4-carboxylate (9_Int-3)

A stirred solution of ethyl 1H-imidazole-4-carboxylate (1.13 g, 8.09 mmol, 1.0 eq) in DMF (11.3 mL, 10V) was prepared and LiHMDS (1M in THF)(8.9 mL, 8.86 mmol, 1.1 eq) was added at −78° C. and stirred for 30 minutes. 2-(4-(bromomethyl)phenyl) acetonitrile (9_Int-2)(1.7 g, 8.09 mmol, 1.0 eq) was added to RM at −78° C. The RM was brought to RT and stirred for 16 h. After completion of the reaction, the RM was quenched in water and extracted by ethyl acetate. The combined organic fractions were dried over Na₂SO₄, concentrated and then purified by flash column chromatography (1.2-2.0% methanol in DCM) to give compound (9_Int-3). MS(ES): 270.23 m/z [M+H]+, LCMS purity: 77.67%, ¹H NMR (400 MHz, DMSO-d6) δ 1.239 (t, J=7.2 Hz, 3H), 4.020 (s, 2H), 4.186 (q, J=7.2 Hz, 2H), 5.240 (s, 2H), 7.348 (s, 4H), 7.877 (s, 1H), 7.929 (s, 1H).

Step-3. N-(4-carbamimidoylbenzyl)-1-(4-(cyanomethyl)benzyl)-1H-imidazole-4-carboxamide (9) was prepared from ethyl 1-(4-(cyanomethyl)benzyl)-1H-imidazole-4-carboxylate (9_Int-3) and 4-(aminomethyl)benzimidamide dihydrochloride in a similar fashion to that described in Example-2. MS(ES): 373.29 m/z [M+H]+, LCMS purity: 98.06%, HPLC purity: 98.03% ¹H NMR (400 MHz, DMSO-d6) δ 4.023 (s, 2H), 4.470 (s, 2H), 5.250 (s, 2H), 7.354 (s, 4H), 7.481 (d, J=6.0 Hz, 2H), 7.755-7.718 (m, 2H), 7.974 (s, 1H), 8.739 (s, 1H), 8.929 (s, 2H), 9.237 (s, 2H).

### Example 16: Preparation of N-(4-carbamimidoylbenzyl)-3-(4-(cyanomethyl)benzyl) isoxazole-5-carboxamide (10)

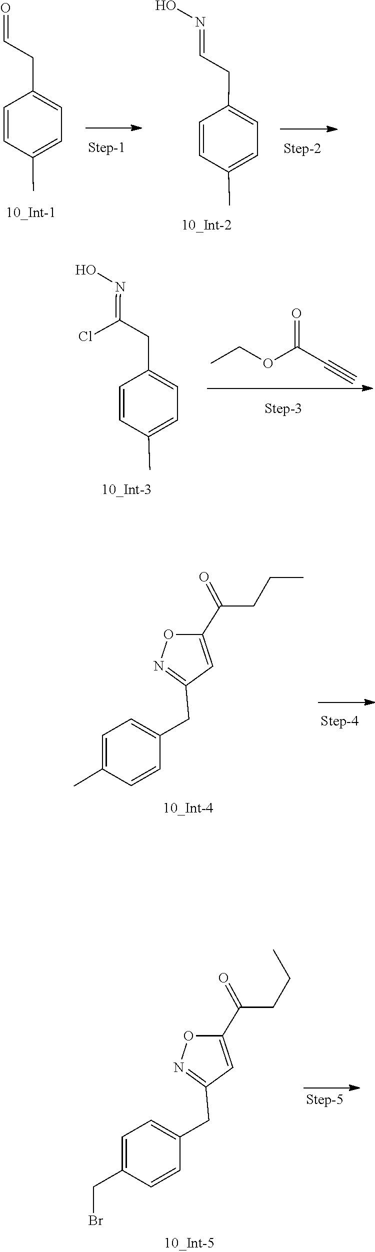

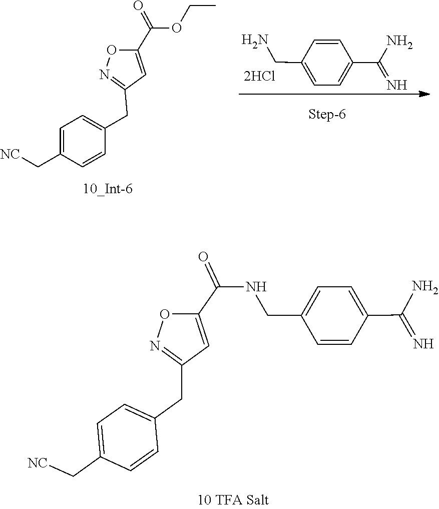 [SMILES: CCOC(=O)c1cc(Cc2ccc(CC#N)cc2)no1 ; N=C(N)c1ccc(CN)cc1 ; N#CCc1ccc(Cc2cc(C(=O)NCc3ccc(C(=N)N)cc3)on2)cc1]

### Step-1. Synthesis of (E)-2-(p-tolyl) acetaldehyde oxime (10_Int-2)

A stirred solution of 2-(p-tolyl) acetaldehyde (2.2 g, 16.39 mmol, 1.0 eq) in DCM (44 mL, 20V) was prepared at RT. Hydroxylamine hydrochloride (2.3 g, 32.79 mmol, 2.0 eq) and TEA (7 g, 68.86 mmol, 4.2 eq) were added at 0° C. The RM was then brought to RT and stirred for 16 h. After completion of the reaction, the reaction mixture was diluted in DCM and washed by 1N HCl in a saturated NaHCO₃ and brine solution. The combined organic fractions were dried over Na₂SO₄; concentrated to give compound (10_Int-2). MS(ES): 150.10 m/z [M+H]+

### Step-2. Synthesis of (Z)—N-hydroxy-2-(p-tolyl)acetimidoyl chloride (10_Int-3)

A stirred solution of (E)-2-(p-tolyl) acetaldehyde oxime (10_Int-2) (4 g, 26.84 mmol, 1.0 eq) in DMF (80 mL, 20V) was prepared and a solution of NCS (3.6 g, 26.84 mmol, 1.0 eq) was added at 0° C. The RM was heated to 50° C. and stirred for 4 h. After completion of reaction, the reaction mixture was evaporated. The remaining residue was quenched in Na₂S₂O₃ solution and extracted by ethyl acetate. The combined organic fractions were dried over Na₂SO₄; concentrated to give compound (10_Int-3). MS(ES): 184.16 m/z [M+H]+, LCMS purity: 60.43%.

### Step-3. Synthesis of ethyl 3-(4-methylbenzyl) isoxazole-5-carboxylate (10_Int-4)

A stirred solution of (Z)—N-hydroxy-2-(p-tolyl)acetimidoyl chloride (10_Int-3)(4 g, 21.78 mmol, 1.0 eq) in diethyl ether (80 mL, 20V) was prepared and TEA (3.3 g, 32.67 mmol, 1.5 eq) followed by ethyl propiolate (2.14 g, 21.78 mmol, 1.0 eq) was added at 0° C. and stirred for 4 h. After completion of the reaction, the RM was filtered. The filtrate was concentrated and then purified by flash column chromatography (1.2-2.0% ethyl acetate in hexane) to give compound (10_Int-4). MS(ES): 246.24 m/z [M+H]+, LCMS purity: 100%, ¹H NMR (400 MHz, DMSO-d6) δ 1.419-1.384 (m, 3H), 2.386 (s, 3H), 4.089 (s, 2H), 4.439-4.403 (m, 2H), 7.211 (s, 4H), 7.410 (s, 1H).

### Step-4. Synthesis of ethyl 3-(4-(bromomethyl)benzyl) isoxazole-5-carboxylate (10_Int-5)

A stirred solution of ethyl ethyl 3-(4-methylbenzyl) isoxazole-5-carboxylate (10_Int-4)(1.8 g, 7.33 mmol, 1.0 eq) was prepared and 1,2 dichloro ethane (54 mL, 30V), Benzoyl peroxide (0.18 g, 0.73 mmol, 0.1 eq), and N-Bromosuccinimide (1.3 g, 1.3 mmol, 1.0 eq) were added at RT. The RM was heated to 90° C. and stirred for 6 h. After completion of the reaction, the reaction mixture was quenched in Na2S2O3 solution and extracted by DCM. The combined organic fractions were dried over Na₂SO₄; concentrated and then purified by flash column chromatography (10-12% ethyl acetate in hexane) to give compound (10_Int-5). MS(ES): 324.2 m/z [M]+326.2 [M+2]+, LCMS purity: 89.94%, ¹H NMR (400 MHz, DMSO-d6) δ 1.286 (t, J=7.2 Hz, 3H), 4.070 (s, 2H), 4.325 (q, J=7.2 Hz, 2H), 4.685 (s, 2H), 7.155 (s, 1H), 7.290 (d, J=8 Hz, 2H), 7.401 (d, J=8 Hz, 2H).

### Step-5. Synthesis of ethyl 3-(4-(cyanomethyl)benzyl) isoxazole-5-carboxylate (10_Int-6)

A stirred solution of ethyl 3-(4-(bromomethyl)benzyl) isoxazole-5-carboxylate (10_Int-5)(0.3 g, 0.92 mmol, 1.0 eq) and acetone (6 mL, 20V) was prepared and tetrabutyl ammoniumcyanide (1 g, 3.7 mmol, 4 eq) was added under nitrogen atmosphere and stirred for 16 h at RT. After completion of the reaction, the RM was quenched in water and extracted by ethyl acetate. The combined organic fractions were dried over Na₂SO₄; concentrated and then purified by flash column chromatography (17-20% ethyl acetate in hexane) to give compound (10_Int-6). MS(ES): 242.42 m/z [M−42]+, LCMS purity: 100%, 1H NMR (400 MHz, DMSO-d6) δ 1.287 (t, J=7.2 Hz, 3H), 4.004 (s, 2H), 4.072 (s, 2H), 4.327 (q, J=7.2 Hz, 2H), 7.133 (s, 1H), 3.343-7.291 (m, 4H).

Step-6. N-(4-carbamimidoylbenzyl)-3-(4-(cyanomethyl)benzyl) isoxazole-5-carboxamide (10) was prepared by ethyl 3-(4-(cyanomethyl)benzyl) isoxazole-5-carboxylate (10_Int-6) and 4-(aminomethyl)benzimidamide dihydrochloride in a similar fashion as that described in Example-2. MS(ES): 374.41 m/z [M+H]+, LCMS purity: 100%, HPLC purity: 99.64% ¹H NMR (400 MHz, DMSO-d6) δ 3.991 (s, 2H), 4.060 (s, 2H), 4.500 (s, 2H), 6.929 (s, 1H), 7.316 (s, 4H), 7.497 (d, J=7.2 Hz, 2H), 7.739 (d, J=7.2 Hz, 2H).

### Example 17: Preparation of N-(4-carbamimidoylbenzyl)-1-((4′-cyano-[1,1′-biphenyl]-4-yl)methyl)-1H-pyrazole-4-carboxamide (13)

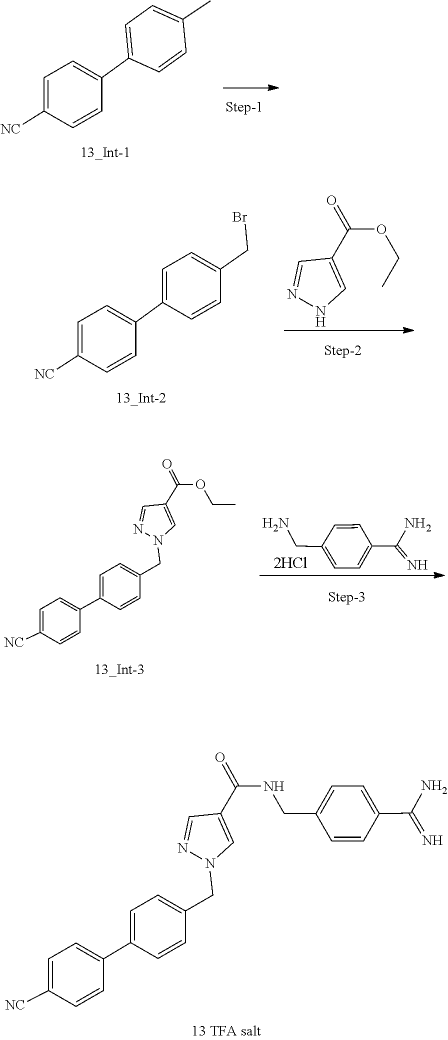

### Step-1. Synthesis of ethyl 3-(4-(bromomethyl)benzyl) isoxazole-5-carboxylate (13_Int-2)

A stirred solution of 4′-methyl-[1,1′-biphenyl]-4-carbonitrile (13_Int-1)(0.1 g, 0.51 mmol, 1.0 eq) and carbon tetrachloride (2 mL, 20V) was prepared. Benzoyl peroxide (0.013 g, 0.05 mmol, 0.1 eq) and N-Bromosuccinimide (0.1 g, 0.56 mmol, 1.1 eq) were then added at RT. The RM was heated to 90° C. and stirred for 16 h. After completion of the reaction, the RM was quenched in Na₂SO₃ solution and extracted by DCM. The combined organic fractions were dried over Na₂SO₄; concentrated and then purified by flash column chromatography (2-5% ethyl acetate in hexane) to give compound (13_Int-2). ¹H NMR (400 MHz, DMSO-d6) δ 4.776 (s, 2H), 7.585 (d, J=8 Hz, 2H), 7.845 (d, J=8 Hz, 2H), 7.947 (s, 4H).

### Step-2. Synthesis of ethyl 1-((4′-cyano-[1,1′-biphenyl]-4-yl)methyl)-1H-pyrazole-4-carboxylate (13_Int-3)

A stirred solution of ethyl 1H-imidazole-4-carboxylate (0.25 g, 1.83 mmol, 1.0 eq) in acetone (10 mL, 20V) were prepared. 4′-(bromomethyl)-[1,1′-biphenyl]-4-carbonitrile (0.5 g, 1.89 mmol, 1.0 eq) and Cs₂CO₃ (1.43 g, 1.40 mmol, 2.4 eq) were added at RT. The RM was heated to 65° C. and stirred for 16 h. After completion of the reaction, the RM was cooled to RT, quenched in water, and extracted by ethyl acetate. The combined organic fractions were dried over Na₂SO₄; concentrated and then purified by flash column chromatography (15-20% ethyl acetate in hexane) to give compound (13_Int-3). MS(ES): 332.23 m/z [M+H]+.

Step-3. N-(4-carbamimidoylbenzyl)-1-((4′-cyano-[1,1′-biphenyl]-4-yl)methyl)-1H-pyrazole-4-carboxamide (13) was prepared from ethyl 1-((4′-cyano-[1,1′-biphenyl]-4-yl)methyl)-1H-pyrazole-4-carboxylate (13_Int-3) and 4-(aminomethyl)benzimidamide dihydrochloride in a similar fashion to that described in Example-2. MS(ES): 435.37 m/z [M+H]+, LCMS purity: 100%, HPLC purity: 99.83% ¹H NMR (400 MHz, DMSO-d6) δ 4.485 (d, J=6 Hz, 2H), 5.424 (s, 2H), 7.340 (d, J=8 Hz, 2H), 7.495 (d, J=8 Hz, 2H), 7.758-7.739 (m, 4H), 7.858 (s, 1H), 7.879 (s, 1H), 7.940-7.920 (m, 3H), 8.329 (s, 1H), 8.794 (t, J=6 Hz, 1H), 8.984 (s, 1H), 9.248 (s, 2H).

### Example 18: Preparation N-(4-carbamimidoylbenzyl)-1-(4-(3-cyanobenzyl)benzyl)-1H-pyrazole-4-carboxamide (28)

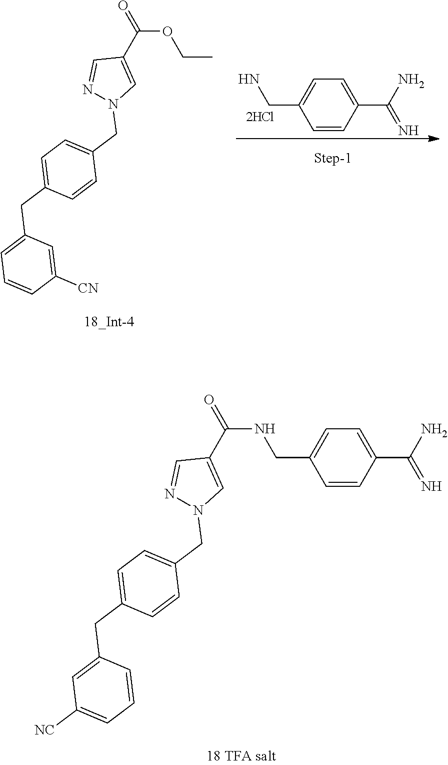 [SMILES: CCOC(=O)c1cnn(Cc2ccc(Cc3cccc(C#N)c3)cc2)c1 ; N=C(N)c1ccc(CN)cc1 ; N#Cc1cccc(Cc2ccc(Cn3cc(C(=O)NCc4ccc(C(=N)N)cc4)cn3)cc2)c1]

Step-1. N-(4-carbamimidoylbenzyl)-1-(4-(3-cyanobenzyl)benzyl)-1H-pyrazole-4-carboxamide (28) was synthesized from ethyl 1-(4-(3-cyanobenzyl)benzyl)-1H-pyrazole-4-carboxylate (28_Int-4) and 4-(aminomethyl)benzimidamide dihydrochloride in a similar fashion to that described in Example-2. MS(ES): 449.34.m/z [M+1]+, LCMS purity: 99.33%, HPLC purity: 100% ¹H NMR (400 MHz, DMSO-d6) δ 3.98 (s, 2H), 4.47 (d, J=6.40 Hz, 2H), 5.30 (s, 2H), 7.23 (q, J=8.8 Hz, 4H), 7.50-7.47 (m, 2H), 7.56 (d, J=Hz, 1H), 7.66 (d, J=7.60 Hz, 1H), 0.00 (d, J=8.4 Hz, 2H), 7.89 (s, 1H), 8.25 (s, 1H), 8.75 (s, 2H), 8.90 (s, 2H), 9.24 (s, 2H).

### Example 19: Preparation of N-(4-carbamimidoylbenzyl)-1-(4-(3-cyano-1H-pyrrol-1-yl)benzyl)-1H-pyrazole-4-carboxamide (25)

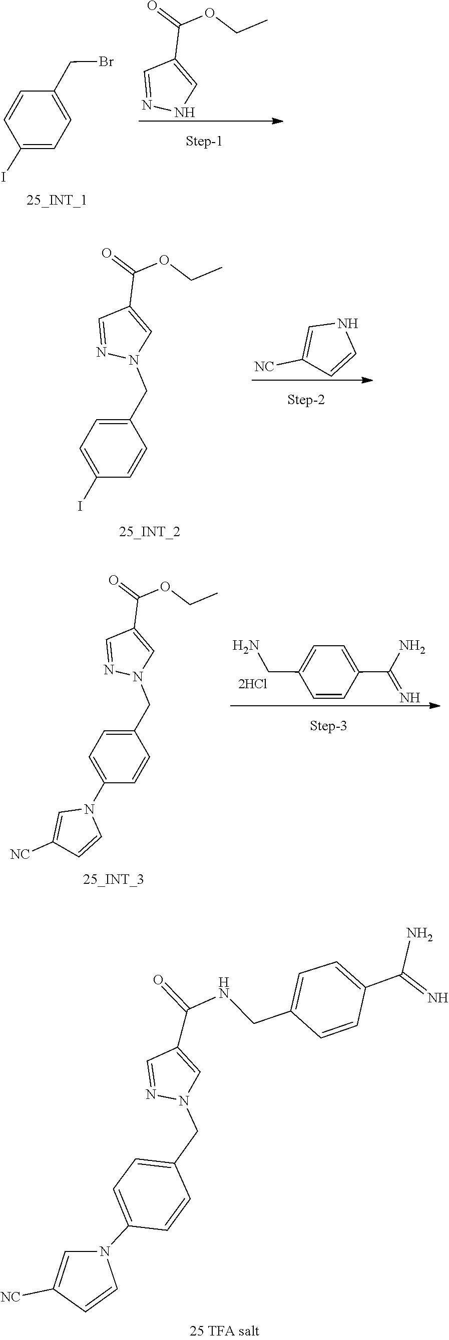

### Step-1. Synthesis of ethyl 1-(4-iodobenzyl)-1H-pyrazole-4-carboxylate (25_Int-2)

A stirred solution of ethyl 1H-pyrazole-4-carboxylate (1.3 g, 9.21 mmol, 1 eq) and 1-(bromomethyl)-4-iodobenzene (25_int-1)(3 g, 10.2 mmol, 1.1 eq) in acetone (13 mL, 10V) was prepared and Cs₂CO₃ (7.2 g, 0.20 mmol, 2.4 eq) was added at RT. The RM was heated to 65° C. and stirred for 8 h. After completion of the reaction, the RM was cooled to RT, quenched in water, and extracted by ethyl acetate. The combined organic fractions were dried over Na₂SO₄; concentrated and then purified by flash column chromatography (30% ethyl acetate in hexanes) to give compound (28_Int-2). MS(ES): 357.27 m/z [M+1]+, LCMS purity: 82%.

### Step-2. Synthesis of ethyl 1-(4-(3-cyano-1H-pyrrol-1-yl)benzyl)-1H-pyrazole-4-carboxylate (25_Int-3)

A stirred solution of ethyl 1-(4-iodobenzyl)-1H-pyrazole-4-carboxylate (28_Int-2). (0.800 g, 2.20 mmol, 1 eq) and 1H-pyrrole-3-carbonitrile (0.426 g, 8.95 mmol, 4 eq) in DMF (8 mL, 10V) was prepared and K₂CO₃ (0.530 g, 6.78 mmol, 3 eq) and CuI (0.426 g, 2.23 mmol, 1 eq) were added at RT. The RM was heated to 100° C. and stirred for 8 h. After completion of the reaction, the RM was cooled at RT, quenched in water, and extracted by ethyl acetate. The combined organic fractions were dried over Na₂SO₄; concentrated and then purified by flash column chromatography (30% ethyl acetate in hexanes) to give compound (25_Int-3). MS(ES): 319.04 m/z [M+1]+, LCMS purity: 70%.

Step-3. N-(4-carbamimidoylbenzyl)-1-(4-(3-cyano-1H-pyrrol-1-yl)benzyl)-1H-pyrazole-4-carboxamide (25) was synthesized from ethyl 1-(4-(3-cyano-1H-pyrrol-1-yl)benzyl)-1H-pyrazole-4-carboxylate (25_Int-3) and 4-(aminomethyl)benzimidamide dihydrochloride in a similar fashion to that described in Example-2. MS(ES): 424.15 m/z [M+1]+, LCMS purity: 100%, HPLC purity: 100% ¹H NMR (400 MHz, DMSO-δ 4.49 (d, J=5.20 Hz, 2H), 5.41 (s, 2H), 6.73 (s, 1H), 7.02 (s, 1H), 7.14 (s, 2H), 7.26 (s, 1H), 7.42 (d, J=8.00 Hz, 1H), 0.00 (t, J=10.8 Hz, 1H), 7.65 (d, J=8.00 Hz, 1H), 7.76 (d, J=7.60 Hz, 1H), 7.94 (s, 1H), 8.23 (s, 1H), 8.32 (s, 1H), 8.79 (s, 1H), 9.10 (s, 2H), 9.25 (s, 2H). 19F NMR 400 MHz, DMSO-d6: δ-73.478 (1F).

### Example 20: Preparation of N-(4-carbamimidoylbenzyl)-1-(4-(4-cyanobenzyl)benzyl)-1H-pyrazole-4-carboxamide (14)

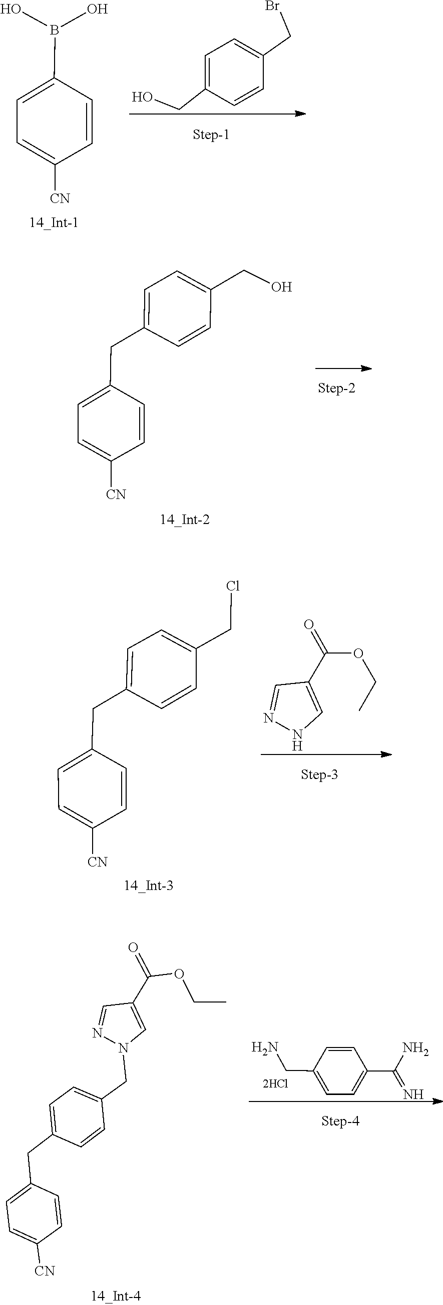

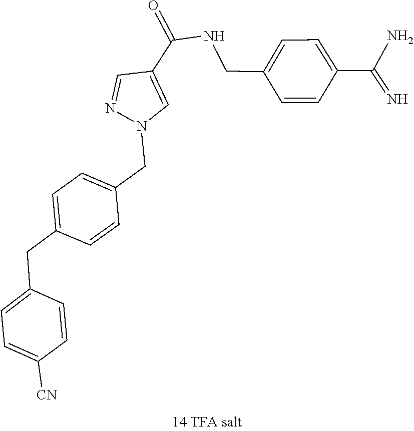 [SMILES: N#Cc1ccc(Cc2ccc(Cn3cc(C(=O)NCc4ccc(C(=N)N)cc4)cn3)cc2)cc1]

### Step-1. Synthesis of 4-(4-(hydroxymethyl)benzyl)benzonitrile (14_Int-2)

A stirred solution (4-cyanophenyl) boronic acid (1.5 g, 7.46 mmol, 1.0 eq)(14_Int-1) in 1,4 dioxane (15 ml, 10V) was prepared and (4-(bromomethyl)phenyl) methanol (1.1 g, 8.20 mmol, 1.1 eq), Cs₂CO₃ (7.27 g, 22.38 mmol, 3 eq), PdCl₂ (dppf). DCM (3.04 g, 3.73 mol, 0.5 eq) and water were added at RT. The RM was heated to 100° C. and stirred for 8 h. After completion of the reaction, the RM was cooled to RT, quenched in water and extracted by ethyl acetate. The combined organic fractions were dried over Na₂SO₄; concentrated and then purified by flash column chromatography (20% ethyl acetate in hexanes) to give compound (14_Int-2). ¹H NMR (400 MHz, DMSO-d6) δ 4.02 (s, 2H), 4.44 (s, 2H), 5.10 (t, J=5.56 Hz, 1H), 7.22 (d, J=8 Hz, 4H), 7.44 (d, J=8.40 Hz, 2H), 7.74 (d, J=8.40 Hz, 2H).

### Step-2. Synthesis of 4-(4-(chloromethyl)benzyl)benzonitrile (14_Int-3)

A stirred solution ethyl 1H-pyrazole-4-carboxylate (0.300 g, 2.14 mmol, 1 eq) in DCM (10 ml, 10V) was prepared and DIPEA (0.115 g, 8.96 mmol, 2 eq) and MsCl (0.107 g, 9.41 mmol, 2.1 eq) were added at 0° C. The RM was stirred at RT for 6 h. After completion of the reaction, the RM was quenched in cold water and extracted by ethyl acetate. The combined organic fractions were dried over Na₂SO₄; concentrated and then purified by flash column chromatography (4% ethyl acetate in hexanes) to give compound (14_Int-3). ¹H NMR (400 MHz, DMSO-d6) δ 400 MHz, DMSO-d6: δ 4.05 (s, 2H), 4.72 (s, 2H), 7.22 (d, J=8 Hz, 4H), 7.44 (d, J=8.40 Hz, 2H), 7.74 (d, J=8.40 Hz, 2H).

### Step-3. Synthesis of ethyl 1-(4-(4-cyanobenzyl)benzyl)-1H-pyrazole-4-carboxylate (25_Int-4)

A stirred solution of ethyl 1H-pyrazole-4-carboxylate (0.300 g, 2.14 mmol, 1 eq) and 4-(4-(chloromethyl)benzyl)benzonitrile (14_Int-3)(0.568 g, 2.35 mmol, 1.1 eq) in acetone (3 mL, 10V) were prepared and Cs₂CO₃ (1.67 g, 5.14 mmol, 2.4 eq) was added at RT. The RM was heated to 65° C. and stirred for 5 h. After completion of the reaction, the RM was cooled at RT, quenched in water and extracted by ethyl acetate. The combined organic fractions were dried over Na₂SO₄; concentrated and then purified by flash column chromatography (16% ethyl acetate in hexanes) to give compound (28_Int-2). MS(ES): 346.3 m/z [M+1]+, LCMS purity: 88%.

Step-4. N-(4-carbamimidoylbenzyl)-1-(4-(4-cyanobenzyl)benzyl)-1H-pyrazole-4-carboxamide (14) was synthesized from ethyl 1-(4-(4-cyanobenzyl)benzyl)-1H-pyrazole-4-carboxylate (25_Int-4) and 4-(aminomethyl)benzimidamide dihydrochloride in a similar fashion to that described in Example-2. MS(ES): 448 m/z [M+1]+, LCMS purity: 100%, HPLC purity: 100% ¹H NMR (400 MHz, DMSO-d6) δ 4.02 (s, 2H), 4.47 (d, J=6.00 Hz, 2H), 5.30 (s, 2H), 7.22 (s, 4H), 7.42 (d, J=8.00 Hz, 2H), 7.49 (d, J=8.40 Hz, 2H), 7.74 (d, J=8.4 Hz, 3H), 0.00 (s, 1H), 8.25 (s, 1H), 8.76 (s, 1H), 8.92 (s, 2H), 8.95 (s, 2H).

### Example 21: Preparation of N-(4-carbamimidoylbenzyl)-1-(4-(2-methoxyethyl)benzyl)-1H-pyrazole-4-carboxamide (15)

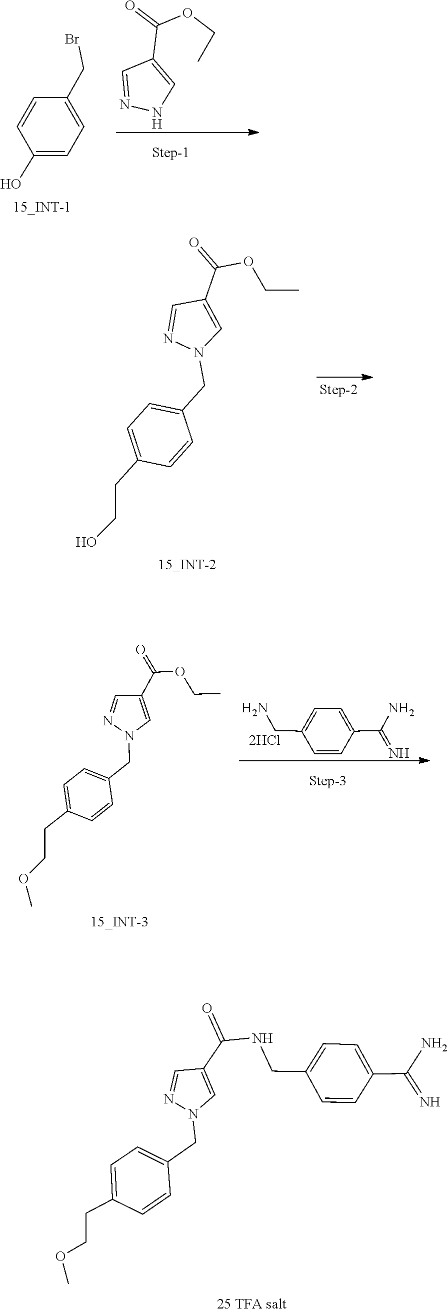

### Step-1. Synthesis of ethyl 1-(4-(2-hydroxyethyl)benzyl)-1H-pyrazole-4-carboxylate (15_Int-2)

A stirred solution ethyl 1H-pyrazole-4-carboxylate (1 g, 7.14 mmol, 1.0 eq)(14_Int-1) in Acetone (10 ml, 10V) was prepared and 2-(4-(bromomethyl)phenyl) ethan-1-ol (1.6 g, 7.85 mmol, 1.1 eq) and Cs₂CO₃ (5.58 g, 17.14 mmol, 2.4 eq) was added at RT. The RM was heated to 65° C. and stirred for 6 h. After completion of the reaction, the RM was cooled to RT, quenched in water and extracted by ethyl acetate. The combined organic fractions were dried over Na₂SO₄; concentrated and then purified by flash column chromatography (25% ethyl acetate in hexanes) to give compound (14_Int-2). MS(ES): 274.2 m/z [M+1]+, LCMS purity: 97%, HPLC purity: % ¹H NMR (400 MHz, DMSO-d6) δ δ 1.22 (t, J=7.20 Hz, 3H), 2.69 (t, J=6.80 Hz, 2H), 3.57 (t, J=6.80 Hz, 2H), 4.21 (t, J=7.20 Hz, 2H), 4.63 (t, J=5.20 Hz, 1H), 5.31 (s, 2H), 7.19 (s, 4H), 0.00 (s, 1H), 8.43 (s, 1H).

### Step-2. Synthesis of ethyl 1-(4-(2-methoxyethyl)benzyl)-1H-pyrazole-4-carboxylate (15_Int-3)

A stirred solution ethyl 1-(4-(2-hydroxyethyl)benzyl)-1H-pyrazole-4-carboxylate (15_Int-2)(0.900 g, 5.17 mmol, 1.0 eq) in DMF (10 ml, 10V) was prepared and NaH (2.85 g, 10.34 mmol, 2 eq) was added and stirred 15 min at 0° C. MeI (0.948 g, 6.724 mmol, 1.3 eq) was then added at 0° C. and the RM was stirred for 3 h. After completion of the reaction, the RM was quenched in cold water and extracted by ethyl acetate. The combined organic fractions were dried over Na₂SO₄; concentrated and then purified by flash column chromatography (12% ethyl acetate in hexanes) to give compound (14_Int-3). MS(ES): 289.26 m/z [M+1]+, 1H NMR (400 MHz, DMSO-d6) δ 400 MHz, DMSO-d6: δ 1.26 (t, J=6.80 Hz, 3H), 2.78 (t, J=6.80 Hz, 2H), 3.27 (s, 3H), 3.51 (t, J=6.80 Hz, 2H), 4.20 (q, J=6.80 Hz, 2H), 5.31 (s, 2H), 7.20 (s, 2H), 0.00 (s, 2H), 8.43 (s, 2H).

Step-3. N-(4-carbamimidoylbenzyl)-1-(4-(2-methoxyethyl)benzyl)-1H-pyrazole-4-carboxamide (15) was synthesized from ethyl 1-(4-(2-methoxyethyl)benzyl)-1H-pyrazole-4-carboxylate (15_Int-3) and 4-(aminomethyl)benzimidamide dihydrochloride in a similar fashion to that described in Example-2. MS(ES): 392.7 m/z [M+1]+, LCMS purity: 100%, HPLC purity: 100% ¹H NMR (400 MHz, DMSO-d6) δ 2.52 (t, J=181.60 Hz, 2H), 3.21 (s, 3H), 3.50 (t, J=6.8 Hz, 2H), 4.48 (d, J=5.60 Hz, 2H), 5.30 (s, 2H), 7.20 (t, J=8.40 Hz, 4H), 7.49 (d, J=8.40 Hz, 2H), 0.00 (d, J=8.00 Hz, 2H), 7.90 (s, 1H), 8.26 (s, 1H), 8.77 (s, 1H), 8.97 (s, 2H), 9.25 (s, 2H).

### Example 22: Preparation of N-(4-carbamimidoyl-3-fluorobenzyl)-1-(4-(3-cyanobenzyl)benzyl)-1H-pyrazole-4-carboxamide (49)

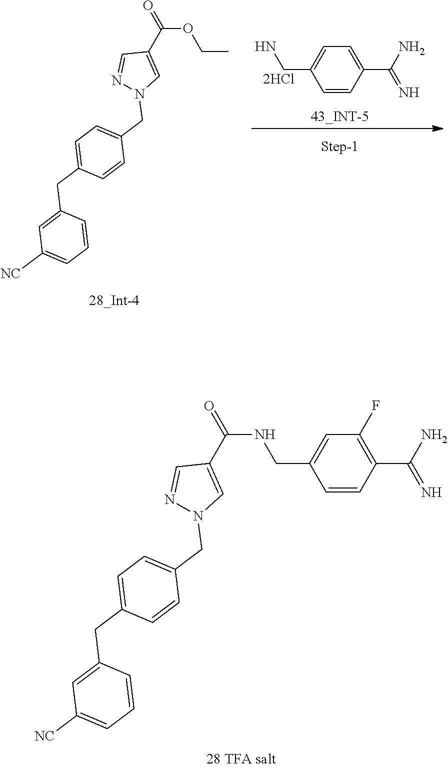

Step 1: Preparation of N-(4-carbamimidoyl-3-fluorobenzyl)-1-(4-(3-cyanobenzyl′1)benzyl)-1H-pyrazole-4-carboxamide (49) was prepared from ethyl 1-(4-(3-cyanobenzyl)benzyl)-1H-pyrazole-4-carboxylate (28_Int-4) and 4-(aminomethyl)-2-fluorobenzimidamide dihydrochloride (43_Int-5) in a similar fashion to that described in Example-2. MS(ES): 467.14 m/z [M+H]+, LCMS purity: 100%, HPLC purity: 100% ¹H NMR 400 MHz, DMSO-d6: δ 3.99 (s, 2H), 4.46 (d, J=6.00 Hz, 2H), 5.34 (s, 2H), 7.34-7.20 (m, 6H), 7.48 (t, J=7.60 Hz, 1H), 7.71-7.56 (m, 3H), 7.89 (s, 1H), 8.25 (s, 1H), 8.76 (t, J=6 Hz, 1H), 9.18 (s, 2H), 9.36 (s, 2H), 19F NMR 400 MHz, DMSO-d6: δ-73.492 (1F), —113.893 (0.35F).

### Example 23: N-(4-carbamimidoyl-2-fluorobenzyl)-1-(4-(3-cyanobenzyl)benzyl)-1H-pyrazole-4-carboxamide (50)

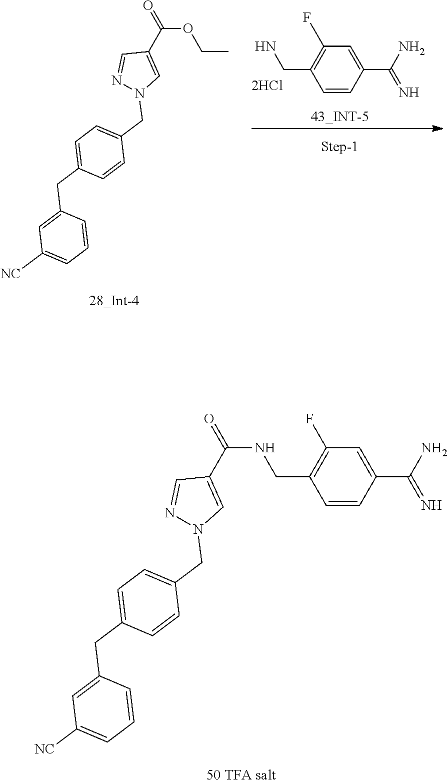

N-(4-carbamimidoyl-2-fluorobenzyl)-1-(4-(3-cyanobenzyl)benzyl)-1H-pyrazole-4-carboxamide (50) was prepared from ethyl 1-(4-(3-cyanobenzyl)benzyl)-1H-pyrazole-4-carboxylate (28_Int-4) and 4-(aminomethyl)-3-fluorobenzimidamide dihydrochloride (45_Int5) in a similar fashion to that described in Example-2. MS(ES): 466.52 m/z [M+H]+, LCMS purity: 100%, HPLC purity: 100% ¹H NMR 400 MHz, DMSO-d6: δ 3.99 (s, 2H), 3.32 (s, 1H), 4.49 (d, J=5.60 Hz, 1H), 5.30 (s, 1H), 7.23 (q, J=8.00 Hz, 5H), 7.71-7.48 (m, 8H), 7.90 (s, 1H), 8.25 (s, 1H), 8.73 (s, 1H), 9.11 (s, 1H), 9.32 (s, 1H), 19F NMR 400 MHz, DMSO-d6: δ-73.536 (1F), −117.182 (0.68F).

### Example 24: Preparation of N-(4-carbamimidoylbenzyl)-1-(3-(2-methoxyethyl)benzyl)-1H-pyrazole-4-carboxamide (69)

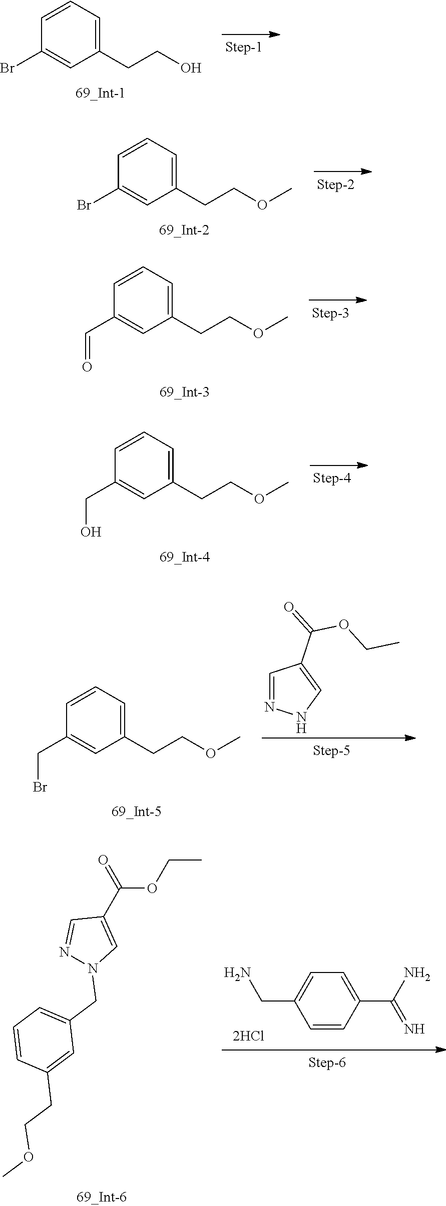

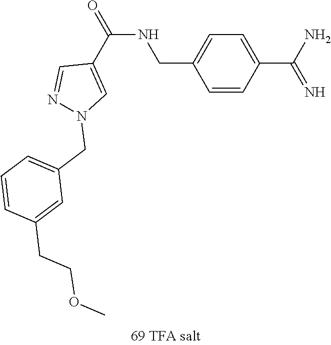 [SMILES: COCCc1cccc(Cn2cc(C(=O)NCc3ccc(C(=N)N)cc3)cn2)c1]

### Step 1. Synthesis of 1-bromo-3-(2-methoxyethyl)benzene (69_Int-2)

A stirred solution of 2-(3-bromophenyl) ethan-1-ol (69_Int-1)(3 g, 14.92 mmol, 1.0 eq) was prepared in DMF (30 mL, 10V) and NaH (60% in mineral oil)(0.72 g, 17.91 mmol, 1.2 eq) was added slowly at 0° C. and stirred for 1 h. Iodomethane (2.54 g, 17.91 mmol, 1.2 eq) was added to the RM at 0° C. and then brought to RT and stirred for 16 h. After completion of the reaction, the RM was slowly quenched with cold water and extracted by ethyl acetate. The combined organic fractions were dried over Na₂SO₄; concentrated and then purified by flash column chromatography (4-5% ethyl acetate in hexanes) to give compound (69_Int-2) MS(ES): 162.23 m/z [M−53]+, LCMS purity: 99.25%, ¹H NMR (400 MHz, DMSO-d6) δ 2.808 (t, J=6.4 Hz, 2H), 3.235 (s, 3H), 3.533 (t, J=6.4 Hz, 2H), 7.250 (d, J=5.2 Hz, 2H), 7.383-7.410 (m, 1H), 7.458 (s, 1H).

### Step 2. Synthesis of 3-(2-methoxyethyl)benzaldehyde (69_Int-3)

A stirred solution of 1-bromo-3-(2-methoxyethyl)benzene (69_Int-2)(1.5 g, 7.0 mmol, 1.0 eq) was prepared in diethylether (15 mL, 10V) and tetramethylethylenediamine (1.75 g, 1.5 mmol, 2.16 eq) was added at RT. The RM was cooled to −75° C. followed by drop wise addition of n-BuL₁ (2.5M in hexane)(5.6 mL, 14.0 mmol, 2.0 eq) under N₂. The RM was stirred at −75° C. for 1 h. The RM was then warmed to −20° C. and stirred for an additional 20 min. The RM was again cooled to −75° C. followed by drop wise addition of dry DMF (7.5 mL, 5V) and stirred for 16 h. After completion of the reaction, the RM was slowly quenched with 1N HCl and extracted by ethyl acetate. The combined organic fractions were dried over Na₂SO₄; concentrated and then purified by flash column chromatography (10-12% ethyl acetate in hexanes) to give compound (69_Int-3) MS(ES): 132.8 m/z [M−32]+, LCMS purity: 100%, 1H NMR (400 MHz, DMSO-d6) δ 2.902 (t, J=6.4 Hz, 2H), 3.325 (s, 3H), 3.581 (t, J=6.4 Hz, 2H), 7.518 (t, J=7.6 Hz, 1H), 7.588 (d, J=8.0 Hz, 1H), 7.742-7.767 (m, 2H), 9.983 (s, 1H).

### Step 3. Synthesis of (3-(2-methoxyethyl)phenyl) methanol (69_Int-4)

A stirred solution of 3-(2-methoxyethyl)benzaldehyde (69_Int-3) (0.23 g, 1.40 mmol, 1.0 eq) was prepared in methanol (4.6 mL, 20V) and sodium borohydride (0.106 g, 2.8 mmol, 2.0 eq) was added slowly at RT and stirred for 4 h. After completion of the reaction, the RM was evaporated. The residue was quenched with water, adjusted to pH ~5 with 2N HCl, and extracted by ethyl acetate. The combined organic fractions were dried over Na₂SO₄; concentrated and then purified by flash column chromatography (12-15% ethyl acetate in hexanes) to give compound (69_Int-4) MS(ES): 149.1 m/z [M−18]+, LCMS purity: 97.53%, ¹H NMR (400 MHz, DMSO-d6) δ 2.794 (t, J=6.8 Hz, 2H), 3.240 (s, 3H), 3.527 (t, J=6.8 Hz, 2H), 4.467 (d, J=5.6 Hz, 2H), 5.150 (t, J=6.0 Hz, 1H), 7.092 (d, J=7.6 Hz, 1H), 7.151 (t, J=7.6 Hz, 2H), 7.231 (t, J=7.6 Hz, 1H).

### Step 4. Synthesis of 1-(bromomethyl)-3-(2-methoxyethyl)benzene (69_Int-5)

A stirred solution of (3-(2-methoxyethyl)phenyl) methanol (69_Int-4)(0.2 g, 1.20 mmol, 1.0 eq) was prepared in DCM (4 mL, 20V). Triphenylphosphine (0.34 g, 1.32 mmol, 1.1 eq) and carbon tetrabromide (0.44 g, 1.32 mmol, 1.1 eq) were then added at RT and stirred for 16 h. After completion of the reaction, the RM was quenched in water and extracted by DCM. The combined organic fractions were dried over Na₂SO₄; concentrated and then purified by flash column chromatography (8-10% ethyl acetate in hexanes) to give compound (69_Int-5) MS(ES): 248.2 m/z [M+18]+, LCMS purity: 100%, ¹H NMR (400 MHz, DMSO-d6) δ 2.816 (t, J=6.8 Hz, 2H), 3.343 (s, 3H), 3.538 (t, J=6.8 Hz, 2H), 4.681 (s, 2H), 7.187 (s, 1H), 7.259-7.309 (m, 2H), 7.332-7.387 (m, 1H).

### Step 5. Synthesis of ethyl 1-(3-(2-methoxyethyl)benzyl)-1H-pyrazole-4-carboxylate (69_Int-6)

A stirred solution of 1-(bromomethyl)-3-(2-methoxyethyl)benzene (69_Int-5)(0.176 g, 0.76 mmol, 1.0 eq) was prepared in acetone (3.52 mL, 20V). Ethyl 1H-pyrazole-4-carboxylate (0.4 g, 0.76 mmol, 1.0 eq) and cesium carbonate (0.6 g, 1.84 mmol, 2.4 eq) were then added at RT. The RM was brought to 65° C. and stirred for 16 h. After completion of the reaction, the RM was filtered and the filtrate was concentrated and then purified by flash column chromatography (20-25% ethyl acetate in hexanes) to give compound (69_Int-6) MS(ES): 289.4 m/z [M+H]+, LCMS purity: 100%, 1H NMR (400 MHz, DMSO-d6) δ 1.25 (t, J=8.0 Hz, 3H), 2.77 (t, J=8.0 Hz, 2H), 3.22 (s, 3H), 3.50 (d, J=8.0 Hz, 2H), 4.12 (q, J=8.0 Hz, 2H), 5.33 (s, 2H), 7.08 (d, J=8.0 Hz, 1H), 7.15-7.18 (m, 2H), 7.25 (t, J=8.0 Hz, 1H), 7.87 (s, 1H), 8.45 (s, 1H).

### Step 6. Synthesis of N-(4-carbamimidoylbenzyl)-1-(3-(2-methoxyethyl)benzyl)-1H-pyrazole-4-carboxamide (69)

A stirred solution of ethyl 1-(3-(2-methoxyethyl)benzyl)-1H-pyrazole-4-carboxylate (69_Int-6)(0.1 g, 0.34 mmol, 1.0 eq) was prepared in THF (1 mL, 10V). 4-(aminomethyl)benzimidamide dihydrochloride (0.09 g, 0.41 mmol, 1.2 eq) and DIPEA (0.18 g, 1.38 mmol, 4.0 eq) were added at RT. The RM was then cooled to 0° C. followed by the addition of TMA (2M in toluene) (1.0 mL, 2.08 mmol, 6.0 eq). The RM was brought to 85° C. and stirred for 16 h. After completion of the reaction, the RM was quenched by 1V of water and evaporated. The residue was washed with 20% methanol in dichloromethane and filtered. The filtrate was concentrated and then purified by PREP HPLC ((A) 0.1% TFA in water (B)₁₀₀% MeCN). Finally, the pure fraction was lyophilized to give compound (69) MS(ES): 392.6 m/z [M+H]+, LCMS purity: 97.92%, HPLC purity: 100% ¹H NMR (400 MHz, DMSO-d6) δ 2.790 (t, J=6.8 Hz, 2H), 3.226 (s, 3H), 3.522 (d, J=6.8 Hz, 2H), 4.492 (d, J=6.4 Hz, 2H), 5.326 (s, 2H), 7.088 (d, J=8.0 Hz, 1H), 7.077-7.131 (m, 2H), 7.181 (t, J=4.4 Hz, 1H), 7.403 (d, J=4.4 Hz, 2H), 7.761 (d, J=8.4 Hz, 2H), 7.924 (s, 1H), 8.274 (s, 1H), 8.776 (t, J=6.0 Hz, 1H), 9.043 (s, 2H), 9.256 (s, 2H).

### Example 25: Preparation of N-(4-carbamimidoyl-3,5-difluorobenzyl)-1-(4-(2-cyanopropan-2-yl)benzyl)-1H-pyrazole-4-carboxamide (44)

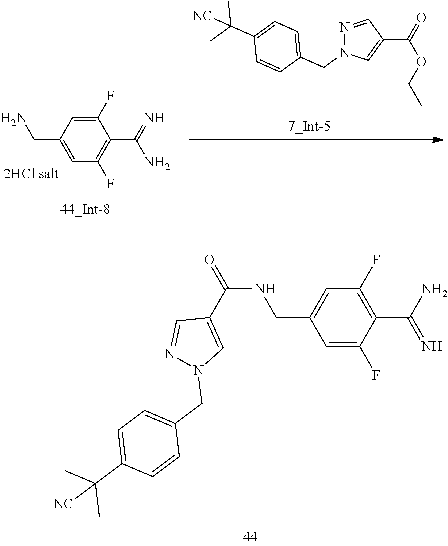 [SMILES: N=C(N)c1c(F)cc(CN)cc1F ; CCOC(=O)c1cnn(Cc2ccc(C(C)(C)C#N)cc2)c1 ; CC(C)(C#N)c1ccc(Cn2cc(C(=O)NCc3cc(F)c(C(=N)N)c(F)c3)cn2)cc1]

### Step-1. Synthesis of N-(4-carbamimidoyl-3,5-difluorobenzyl)-1-(4-(2-cyanopropan-2-yl)benzyl)-1H-pyrazole-4-carboxamide (44)

A stirred solution of ethyl 1-(4-(2-cyanopropan-2-yl)benzyl)-1H-pyrazole-4-carboxylate (7_Int-5)(0.1 g, 0.3367 mmol, 1.0 eq) in toluene (2 mL, 10V) was prepared. 4-(aminomethyl)-2,6-difluorobenzimidamide dihydrochloride (44_Int-8)(0.095 g, 0 . . . 505 mmol, 1.5 eq) and DIPEA (0.6 mL, 0.8417 mmol, 2.5 eq) were added to the RM at RT. The RM was cooled to 0° C. and TMA (2.0M in toluene, 0.6 mL, 1.0101 mmol, 3.0 eq) was added. The RM was then heated to 100° C. and stirred for 16 h. After completion of the reaction, the RM was quenched by 1V of water and evaporated. The residue was washed with 20% methanol in dichloromethane and filtered. Solid residue was discarded and the filtrate was concentrated and then purified by PREP HPLC ((A)_{0.1}% TFA in water (B)₁₀₀% MeCN). Finally, the pure fraction was lyophilized to give compound (44). MS(ES): 437.21 m/z [M+1]+, LCMS purity: 100%, HPLC purity: 94.56% 1H 400 MHz, DMSO-d6: δ 1.664 (s, 6H), 4.462 (d, J=6.40 Hz, 2H), 5.369 (s, 2H), 7.252 (d, J=8.8 Hz, 2H), 7.331 (d, J=8.0 Hz, 2H), 7.511 (d, J=8.00 Hz, 2H), 7.921 (s, 1H), 8.310 (s, 1H), 8.840 (t, J=6.4 Hz, 1H), 9.513 (s, 2H), 9.669 (s, 2H).

### Example 26: N-(4-carbamimidoylbenzyl)-1-(4-(2-cyanopropan-2-yl)benzyl)-5-cyclopropyl-1H-pyrazole-4-carboxamide (83)

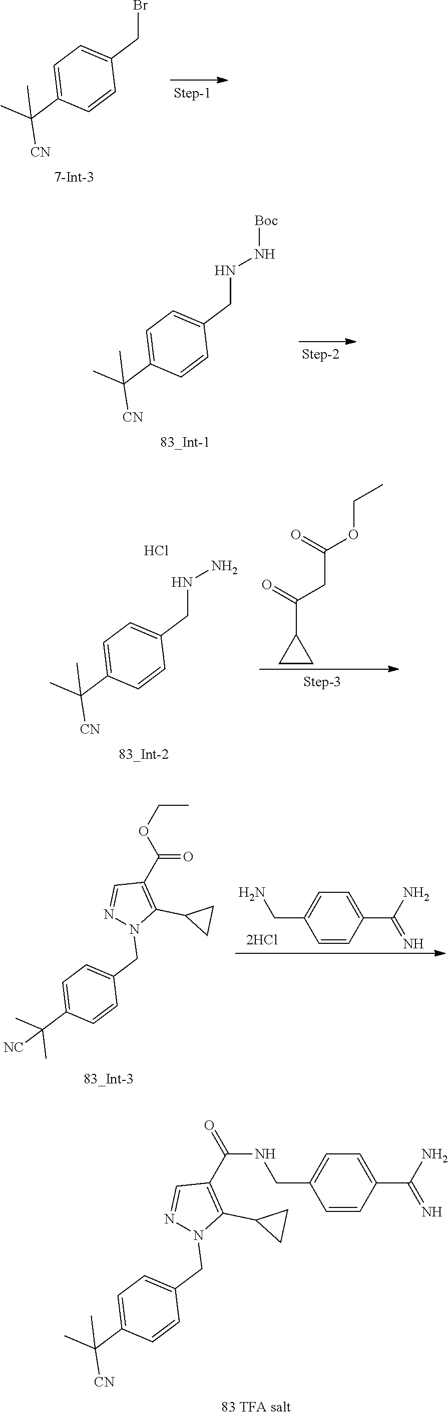

### Step-1. Synthesis of tert-butyl 2-(4-(2-cyanopropan-2-yl)benzyl) hydrazine-1-carboxylate (83_Int-1)

A stirred solution of 2-(4-(bromomethyl)phenyl)-2-methylpropanenitrile (7_Int-3)(0.5 g, 2.1 mmol, 1.0 eq) was prepared in dimethylacetamide (5 mL, 10V). DIPEA (0.54 g, 4.2 mmol, 2.0 eq) and t-butyl carbazate (0.55 g, 4.2 mmol, 2.0 eq) was added at RT and stirred at 70° C. for 16 h. After completion of the reaction, the RM was quenched in water and extracted by ethyl acetate. The combined organic fractions were washed by brine solution, dried over Na₂SO₄, concentrated, and purified by flash column chromatography (10-12% ethyl acetate in hexanes) to give compound (83_Int-1), 1H 400 MHz, DMSO-d6: δ 1.637 (s, 6H), 1.760 (s, 9H), 4.206 (s, 2H), 7.514 (s, 4H), 7.744 (d, br, J=8.00 Hz, 1H), 7.886-8.004 (d, br, J=47.2, 1H).

### Step-2. Synthesis of 2-(4-(hydrazineylmethyl)phenyl)-2-methylpropanenitrile (83_Int-2)

A stirred solution of tert-butyl 2-(4-(2-cyanopropan-2-yl)benzyl) hydrazine-1-carboxylate (83_Int-1)(2.0 g, 6.9 mmol, 1.0 eq) was prepared in methanol (10 mL, 5V) and water (22 mL, 11V) and concentrated HCl (6.6 mL, 3.3V) was added at RT. The RM was heated to 80° C. and stirred for 16 h. After completion of the reaction, the RM was evaporated and triturated in ethyl acetate to give compound (83_Int-2). MS(ES): 191.4 m/z [M+H]+, LCMS purity: 83.16%, 1H 400 MHz, DMSO-d6: δ 1.693 (s, 6H), 4.076 (s, 2H), 7.096 (s, br, 2H), 7.517 (d, J=12.8 Hz, 4H).

### Step-3. Synthesis of ethyl 1-(4-(2-cyanopropan-2-yl)benzyl)-5-cyclopropyl-1H-pyrazole-4-carboxylate (7_Int-3)

A solution of ethyl 3-cyclopropyl-3-oxopropanoate (0.22 g, 1.41 mmol, 1.0 eq) and dimethylformamide dimethyl acetal (0.185 g, 1.55 mmol, 1.1 eq) was prepared and stirred at 75° C. for 90 min. The RM was cooled to RT followed by addition of ethanol (4.4 mL, 20V), TEA (0.57 g, 5.63 mmol, 4.0 eq) and 2-(4-(hydrazinylmethyl)phenyl)-2-methylpropanenitrile (83_Int-2)(0.365 g, 1.41 mmol, 1.0 eq). The RM was heated to 80° C. and stirred for 16 h. After completion of the reaction, the RM was quenched in water and extracted by ethyl acetate. The combined organic fractions were washed by brine solution, dried over Na₂SO₄, concentrated, and purified by flash column chromatography (12-15% ethyl acetate in hexanes) to give compound (83_Int-3). MS(ES): 180.88 m/z [M+H]+, LCMS purity: 89%, 1H 400 MHz, DMSO-d6: δ 1.637 (s, 6H), 4.174-4.225 (m, 2H), 5.484 (s, 2H), 7.222 (d, J=8.0 Hz, 2H), 7.505 (d, J=8.0 Hz, 2H), 7.830 (s, 1H).

### Step-4. Synthesis of N-(4-carbamimidoylbenzyl)-1-(4-(2-cyanopropan-2-yl)benzyl)-5-cyclopropyl-1H-pyrazole-4-carboxamide (83)

The final compound was prepared from ethyl 1-(4-(2-cyanopropan-2-yl)benzyl)-5-cyclopropyl-1H-pyrazole-4-carboxylate (7_Int-3) and 4-(aminomethyl)benzimidamide dihydrochloride in a similar fashion to that described in (Example 24)(83). MS(ES): 441.4 m/z [M+H]+, LCMS purity: 100%, HPLC purity: 100% 1H 400 MHz, DMSO-d6: δ 0.734-0.757 (m, 2H), 0.919-0.967 (m, 2H), 1.658 (s, 6H), 1.753-1.796 (m, 1H), 4.472 (d, J=6.0 Hz, 2H), 5.438 (s, 2H), 7.227 (d, J=8 Hz, 2H), 7.501 (t, J=8.8 Hz, 4H), 7.762 (d, J=8.0 Hz, 2H), 7.801 (s, 1H), 8.534 (t, J=6.0 Hz, 1H), 8.977 (s, 2H), 9.258 (s, 2H).

### Example 27: N-(4-carbamimidoylbenzyl)-1-(4-(2-cyanopropan-2-yl)benzyl)-5-propyl-1H-imidazole-4-carboxamide (84)

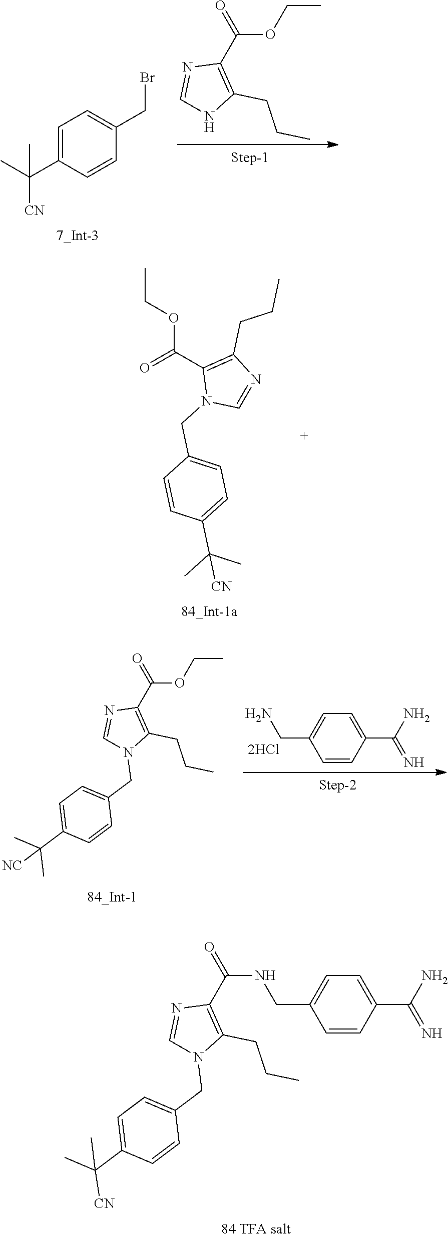

### Step-1. Synthesis of ethyl 1-(4-(2-cyanopropan-2-yl)benzyl)-5-propyl-1H-imidazole-4-carboxylate (84_Int-1)

A stirred solution of ethyl 5-propyl-1H-imidazole-4-carboxylate (0.45 g, 2.47 mmol, 1.0 eq) in DMF (4.5 mL, 10V) was prepared and NaHMDS (1M in THF, 2.5 mL, 2.47 mmol, 1.0 eq) was added at 0° C. and stirred for 30 min. 2-(4-(bromomethyl)phenyl)-2-methylpropanenitrile (7_Int-3)(0.5 g, 3.56 mmol, 1.0 eq) was added to the RM at 0° C., brought to RT, and stirred for 16 h. After completion of the reaction, the RM was quenched in water and extracted by ethyl acetate. The combined organic fractions were dried over Na₂SO₄; concentrated and then purified by flash column chromatography (20-25% ethyl acetate in hexanes). (84_Int-1). Confirmed by 2D NMR (ROE analysis). MS(ES): 339.9 m/z [M+H]+, LCMS purity: 84.72%, 1H 400 MHz, DMSO-d6: δ 0.807 (t, J=7.2 Hz, 3H), 1.251 (q, J=8.0 Hz, 3H), 1.297 (s, 2H), 1.654 (s, 6H), 2.734 (q, J=6.4 Hz, 2H), 4.186 (q, J=7.2 Hz, 2H), 5.264 (s, 2H), 7.194 (d, J=8.0 Hz, 2H), 7.512 (d, J=8.0 Hz, 2H), 7.788 (s, 1H).

### Step-2. Synthesis of N-(4-carbamimidoylbenzyl)-1-(4-(2-cyanopropan-2-yl)benzyl)-5-propyl-1H-imidazole-4-carboxamide (84)

The final compound was prepared from ethyl 1-(4-(2-cyanopropan-2-yl)benzyl)-5-propyl-1H-imidazole-4-carboxylate (84_Int-1) and 4-(aminomethyl)benzimidamide dihydrochloride in a similar fashion to that described in (Example-24)(84). MS(ES): 443.35 m/z [M+H]+, LCMS purity: 100%, HPLC purity: 99.63% 1H 400 MHz, DMSO-d6: δ 0.782 (t, J=7.2 Hz, 3H), 1.262-1302 (m, 2H), 1.656 (d, J=6.00 Hz, 2H), 2.773 (t, J=8 Hz, 2H), 4.473 (d, J=6.4 Hz, 2H), 5.260 (s, 2H), 7.226 (d, J=8.4 Hz, 2H), 7.487-7.526 (m, 4H), 7.745 (d, J=8.4 Hz, 2H), 7.872 (s, 1H), 8.634 (t, J=6.0 Hz, 1H), 9.069 (s, 2H), 9.248 (s, 2H).

### Example 28: Preparation of N-(4-carbamimidoylbenzyl)-1-(4-(2-cyanopropan-2-yl)benzyl)-5-methyl-1H-pyrazole-4-carboxamide (81)

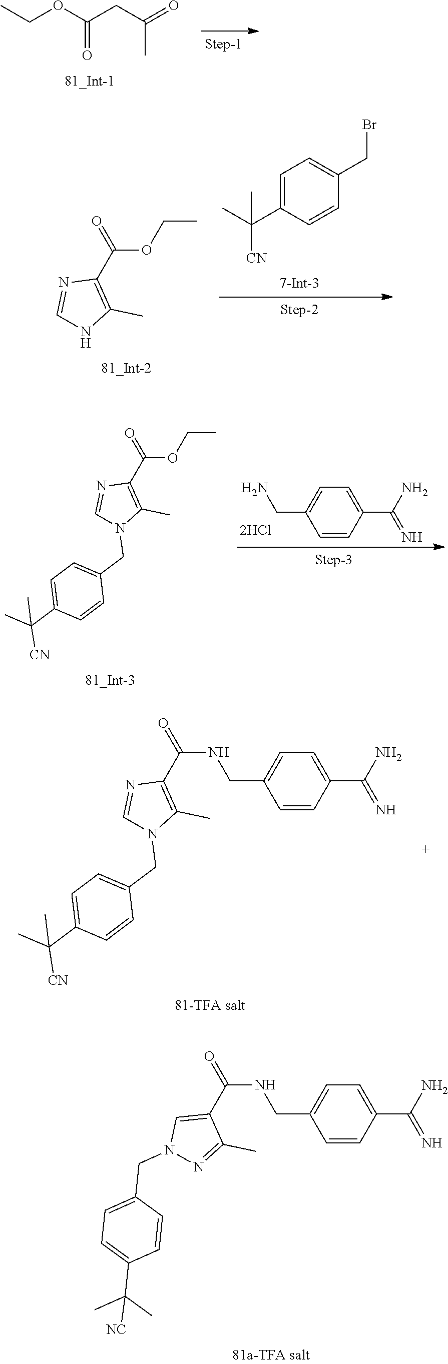

### Step-1. Synthesis of ethyl 5-methyl-1H-pyrazole-4-carboxylate (81_Int-2)

A solution of ethyl 3-oxobutanoate (81_Int-1)(0.5, 3.84 mmol, 1.0 eq) and dimethylformamide dimethyl acetal (0.46 g, 3.84 mmol, 1.0 eq) was prepared and stirred at 110° C. for 1 h. The RM was cooled to RT followed by addition of ethanol (5.0 mL, 10V) and hydrazine hydrate (99%)(0.2 g, 3.84 mmol, 1.0 eq). The RM was heated to 70° C. and stirred for 2 h. After completion of the reaction, the reaction mixture was evaporated to give compound (81_Int-2). MS(ES): 154.96 m/z [M+H]+, LCMS purity: 100%, ¹H NMR (400 MHz, DMSO-d6) δ 1.241 (t, J=8.00 Hz, 3H), 2.446 (s, 3H), 4.178 (q, J=4.2 Hz, 2H), 7.794 (s, 1H), 13.102 (s, 1H).

### Step-2. Synthesis of ethyl 1-(4-(2-cyanopropan-2-yl)benzyl)-5-methyl-1H-pyrazole-4-carboxylate (81_Int-3)

A stirred solution of ethyl 5-methyl-1H-pyrazole-4-carboxylate (81_Int-2)(0.25 g, 1.62 mmol, 1.0 eq) in DMF (2.5 mL, 10V) was prepared and NaHMDS (1M in THF)(1.62 mL, 1.62 mmol, 1.0 eq) was added at 0° C. and stirred for 30 min . . . 2-(4-(bromomethyl)phenyl)-2-methylpropanenitrile (7_Int-3) (0.4 g, 1.62 mmol, 1.0 eq) was added to the RM at 0° C. The RM was the brought to RT and stirred for 16 h. After completion of the reaction, the RM was quenched in water and extracted by ethyl acetate. The combined organic fractions were dried over Na₂SO₄; concentrated and then purified by flash column chromatography (20-25% ethyl acetate in hexanes) to give compound (81_Int-3). MS(ES): 312.2 m/z [M+H]+, LCMS purity: 100%, 1H 400 MHz, DMSO-d6: δ 1.239 (t, J=8.00 Hz, 3H), 1.656 (s, 6H), 2.299 (s, 3H), 4.171 (t, J=8.00 Hz, 2H), 5.273 (s, 2H), 7.190 (d, J=8.00 Hz, 2H), 7.322 (d, J=8.00 Hz, 2H), 7.829 (s, 1H).

### Step-3. Synthesis of N-(4-carbamimidoylbenzyl)-1-(4-(2-cyanopropan-2-yl)benzyl)-5-propyl-1H-imidazole-4-carboxamide (81)

The final compound was prepared from ethyl 1-(4-(2-cyanopropan-2-yl)benzyl)-5-propyl-1H-imidazole-4-carboxylate (81_Int-3) and 4-(aminomethyl)benzimidamide dihydrochloride in a similar fashion to that described in (Example 25)(81). MS(ES): 415.35 m/z [M+H]+, LCMS purity: 100%, HPLC purity: 99.47% 1H 400 MHz, DMSO-d6: δ 1.655 (s, 6H), 2.457 (s, 3H), 4.777 (d, J=6.0 Hz, 2H), 5.341 (s, 2H), 7.191 (d, J=8 Hz, 2H), 7.515-7.477 (m, 4H), 7.750 (d, J=8 Hz, 2H), 7.985 (s, 1H), 8.676 (t, J=6 Hz, 1H), 8.886 (s, 2H), 9.247 (s, 2H).

### Example 29: N-(4-carbamimidoyl-3-fluoro-5-methoxybenzyl)-1-(4-(2-cyanopropan-2-yl)benzyl)-1H-pyrazole-4-carboxamide (48)

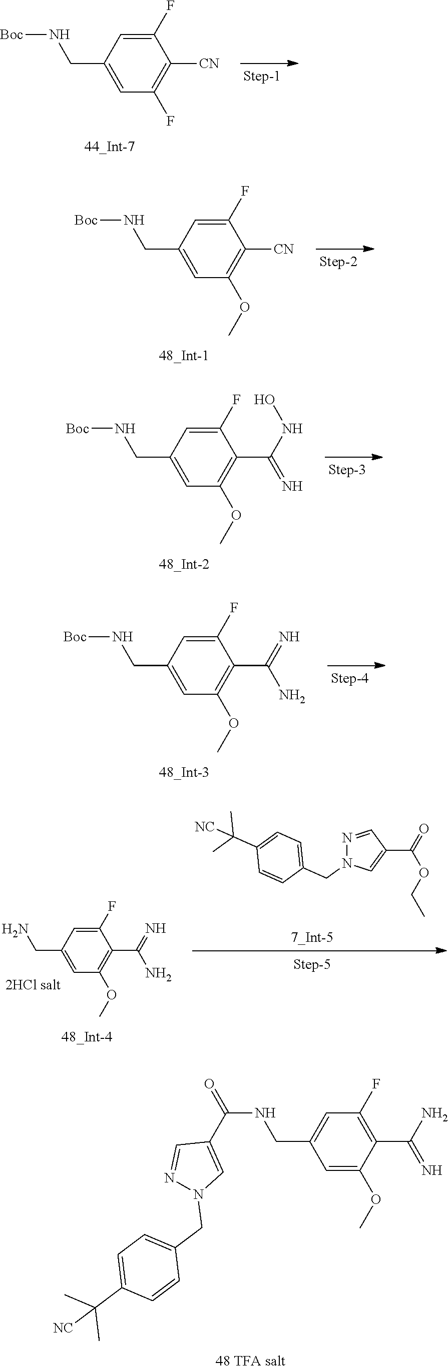

### Step-1. Synthesis of tert-butyl (4-cyano-3-fluoro-5-methoxybenzyl) carbamate (48_Int-1)

A stirred solution of tert-butyl (4-cyano-3,5-difluorobenzyl) carbamate (44_Int-7)(5 g, 18.36 mmol, 1.0 eq) was prepared in THF (50 mL, 10V) and MeOH (50 mL, 10V). The RM was cooled to 0° C. and sodium methoxide (4.02 g, 74.6 mmol, 4.0 eq) was slowly added. The RM was brought to RT and stirred for 5 h. After completion of the reaction, the RM was quenched in cold water and extracted by ethyl acetate. The combined organic fractions were dried over Na₂SO₄; concentrated to give compound (48_Int-1) 1H 400 MHz, DMSO-d6: δ 1.631 (s, 9H), 3.924 (s, 3H), 4.169 (d, J=6.0 Hz, 2H), 6.871 (d, J=10 Hz, 1H), 6.982 (s, 1H), 7.544 (t, J=6.0 Hz, 1H).

### Step-2. Synthesis of tert-butyl (3-fluoro-4-(N-hydroxycarbamimidoyl)-5-methoxybenzyl) carbamate (48_Int-2)

A stirred solution of tert-butyl (4-cyano-3-fluoro-5-methoxybenzyl) carbamate (48_Int-1)(4.7 g, 16.78 mmol, 1.0 eq) was prepared in MeOH (50 mL, 10V). Hydroxylamine hydrochloride (1.96 μm, 28.536 mmol, 1.7 eq) and DIPEA (5.0 ml, 28.535 mmol, 1.7 eq) were then added at RT. The RM was heated to 70° C. and stirred for 16 h. After completion of the reaction, the RM was cooled to RT, quenched in water, and extracted by DCM. The combined organic fractions were dried over Na₂SO₄; concentrated to give compound (48_Int-2). MS(ES): 314.29 m/z [M+1]+, LCMS purity: 80%.

### Step-3. Synthesis of tert-butyl (4-carbamimidoyl-3-fluoro-5-methoxybenzyl) carbamate (48_Int-3)

A stirred solution of tert-butyl (3-fluoro-4-(N-hydroxycarbamimidoyl)-5-methoxybenzyl) carbamate (48_Int-2)(2 g, 6.3397 mmol, 1.0 eq) was prepared in MeOH. (20 mL, 10V) Ammonium chloride (1.7 g, 31.9485 mmol, 5.0 eq) and iron (1.7 g, 31.9485 mmol, 5.0 eq) were then added at RT. The RM was cooled to 0° C. followed by dropwise addition of acetic acid (20 mL, 5V). The RM was heated to 70° C. and stirred for 16 h. After completion of the reaction, the RM was cooled to RT. The RM was concentrated, quenched in cold water, and basified by slow addition of saturated NaOH solution to a target pH of 10. The solids were filtered and the filtrate was extracted by DCM. The combined organic fractions were dried over Na₂SO₄; concentrated to give compound (48_Int-3). MS(ES): 298.23 m/z [M+1]+, LCMS purity: 73.2%.

### Step-4. Synthesis of 4-(aminomethyl)-2-fluoro-6-methoxybenzimidamide (48_Int-4)

A stirred solution of tert-butyl (4-carbamimidoyl-3-fluoro-5-methoxybenzyl) carbamate (48_Int-3)(0.8 g, 25.473 mmol, 1.0 eq) was prepared in water (8 mL, 10V) and concentrated HCl (2.6 mL, 3.3V) was added at rt and stirred for 3 h. After completion of the reaction, the RM was concentrated and triturated in methanol to give compound (43_Int-4). MS(ES): 198.2 m/z [M+1]+, LCMS purity: 36%.

### Step-5. Synthesis of N-(4-carbamimidoyl-3-fluoro-5-methoxybenzyl)-1-(4-(2-cyanopropan-2-yl)benzyl)-1H-pyrazole-4-carboxamide (48)

The final compound was prepared from ethyl 1-(4-(2-cyanopropan-2-yl)benzyl)-1H-pyrazole-4-carboxylate (7_Int-5) and 4-(aminomethyl)-2-fluoro-6-methoxybenzimidamide dihydrochloride (48_Int-4) in a similar fashion to that described in (Example-25)(48). MS(ES): 449.22 m/z [M+1]+, LCMS purity: 93.36%, HPLC purity: 93.46% 1H 400 MHz, DMSO-d6: δ 1.660 (s, 6H), 3.843 (s, 3H), 4.436 (d, J=5.60 Hz, 2H), 5.364 (s, 2H), 6.878 (d, J=10 Hz, 2H), 6.966 (s, 1H), 7.324 (d, J=8 Hz, 2H), 7.507 (d, J=8.0 Hz, 2H), 7.919 (s, 1H), 8.299 (s, 1H), 8.778 (t, J=6.4 Hz, 1H), 9.139 (s, 2H), 9.383 (s, 2H).

### Example 30: N-(4-carbamimidoyl-3-fluorobenzyl)-1-(4-(cyanomethyl)benzyl)-1H-pyrazole-4-carboxamide (72)

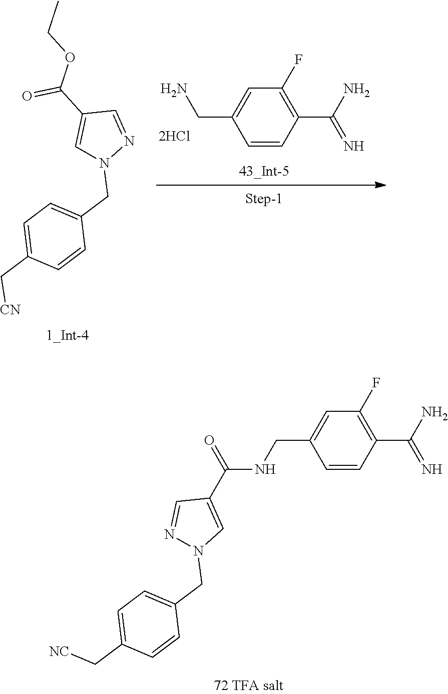 [SMILES: CCOC(=O)c1cnn(Cc2ccc(CC#N)cc2)c1 ; N=C(N)c1ccc(CN)cc1F ; N#CCc1ccc(Cn2cc(C(=O)NCc3ccc(C(=N)N)c(F)c3)cn2)cc1]

### Step-1. Synthesis of N-(4-carbamimidoyl-3-fluorobenzyl)-1-(4-(cyanomethyl)benzyl)-1H-pyrazole-4-carboxamide (72)

The final compound was prepared from ethyl 1-(4-(cyanomethyl)benzyl)-1H-pyrazole-4-carboxylate (1_Int-4) and 4-(aminomethyl)-2-fluorobenzimidamide dihydrochloride (43_Int-5) in a similar fashion to that described in (Example-25)(72). MS(ES): 391.22 m/z [M+1]+, LCMS purity: 100%, HPLC purity: 100% 1H 400 MHz, DMSO-d6: δ 4.024 (s, 2H), 4.460 (s, 2H), 5.357 (s, 2H), 7.304-3.349 (m, 6H), 7.622 (s, 1H), 7.915 (s, 1H), 8.280 (s, 1H), 8.800 (t, J=6.4 Hz, 1H), 9.132 (s, 2H), 9.377 (s, 2H).

### Example 31: N-(4-carbamimidoyl-3-fluorobenzyl)-1-(4-(2-methoxyethyl)benzyl)-1H-pyrazole-4-carboxamide (73)

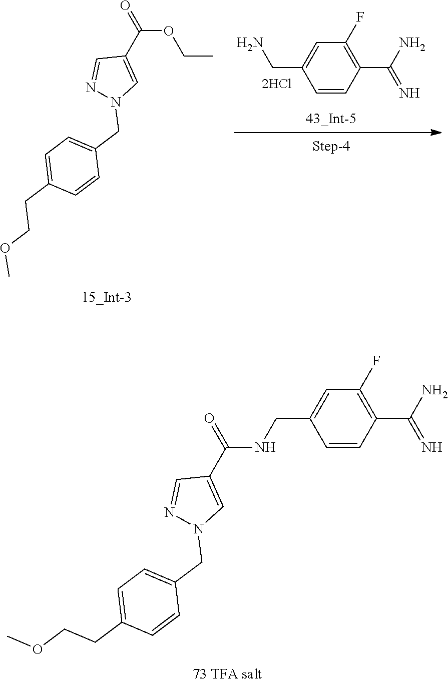 [SMILES: CCOC(=O)c1cnn(Cc2ccc(CCOC)cc2)c1 ; N=C(N)c1ccc(CN)cc1F ; COCCc1ccc(Cn2cc(C(=O)NCc3ccc(C(=N)N)c(F)c3)cn2)cc1]

### Step-1. Synthesis of N-(4-carbamimidoyl-3-fluorobenzyl)-1-(4-(2-methoxyethyl)benzyl)-1H-pyrazole-4-carboxamide (73)

The final compound was prepared from ethyl 1-(4-(2-methoxyethyl)benzyl)-1H-pyrazole-4-carboxylate (15_Int-3) and 4-(aminomethyl)-2-fluorobenzimidamide dihydrochloride (43_Int-5) in a similar fashion to that described in (Example-25)(73). MS(ES): 410.4 m/z [M+1]+, LCMS purity: 100%, HPLC purity: 99.67% 1H 400 MHz, DMSO-d6: δ 2.774 (t, J=6.80 Hz, 2H), 3.212 (s, 3H), 3.500 (t, J=6.80 Hz, 2H), 7.460 (d, J=6.0 Hz, 2H), 5.303 (s, 2H), 7.175-7.224 (m, 4H), 7.319 (t, J=7.2 Hz, 2H), 7.899 (s, 1H), 8.260 (s, 1H), 8.797 (t, J=6.0 Hz, 1H), 9.182 (s, 2H), 9.377 (s, 2H).

### Example 32: N-(4-carbamimidoyl-3-fluorobenzyl)-1-(4-(cyanomethyl)benzyl)-5-(methoxymethyl)-1H-pyrazole-4-carboxamide (75)

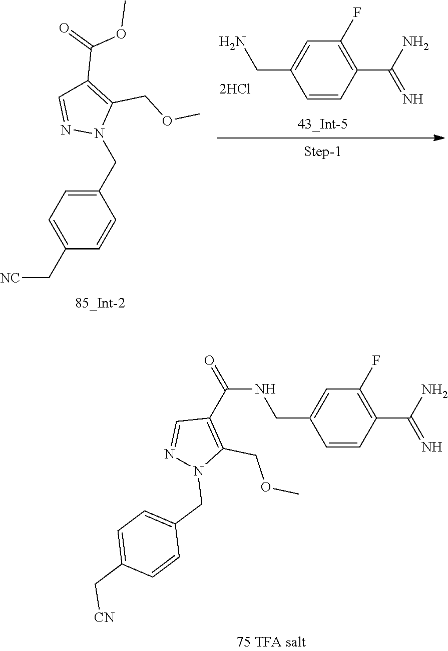 [SMILES: COCc1c(C(=O)OC)cnn1Cc1ccc(CC#N)cc1 ; N=C(N)c1ccc(CN)cc1F ; COCc1c(C(=O)NCc2ccc(C(=N)N)c(F)c2)cnn1Cc1ccc(CC#N)cc1]

### Step-1. Synthesis of N-(4-carbamimidoyl-3-fluorobenzyl)-1-(4-(cyanomethyl)benzyl)-5-(methoxymethyl)-1H-pyrazole-4-carboxamide (75)

The final compound was prepared from methyl 1-(4-(cyanomethyl)benzyl)-5-(methoxymethyl)-1H-pyrazole-4-carboxylate (85_Int-2) and 4-(aminomethyl)-2-fluorobenzimidamide dihydrochloride (43_Int-5) in a similar fashion to that described in (Example-24)(75). MS(ES): 435.4 m/z [M+1]+, LCMS purity: 100%, HPLC purity: 100% 1H 400 MHz, DMSO-d6: δ 3.232 (s, 3H), 4.019 (s, 2H), 4.85 (d, J=6.0 Hz, 2H), 4.812 (s, 2H), 5.365 (s, 2H), 7.242 (d, J=7.6 Hz, 2H), 7.307-7.374 (M, 4H), 7.640 (t, =Hz, 1H), 8.039 (s, 1H), 8.871 (t, J=6.0 Hz, 1H), 9.197 (s, 2H), 9.401 (s, 2H).

### Example 33: N-(4-carbamimidoylbenzyl)-1-(4-(methoxymethyl)benzyl)-1H-pyrazole-4-carboxamide (71)

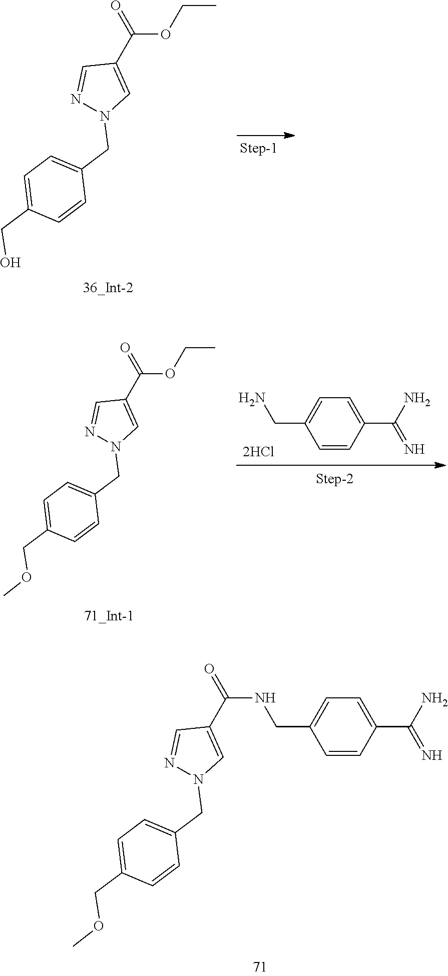 [SMILES: CCOC(=O)c1cnn(Cc2ccc(CO)cc2)c1 ; CCOC(=O)c1cnn(Cc2ccc(COC)cc2)c1 ; N=C(N)c1ccc(CN)cc1 ; COCc1ccc(Cn2cc(C(=O)NCc3ccc(C(=N)N)cc3)cn2)cc1]

### Step-1. Synthesis of ethyl 1-(4-(methoxymethyl)benzyl)-1H-pyrazole-4-carboxylate (71_Int-1)

A stirred solution ethyl 1-(4-(hydroxymethyl)benzyl)-1H-pyrazole-4-carboxylate (36_Int-2)(2.5 g, 9.6153 mmol, 1.0 eq) was prepared in in DMF (25 ml, 10V) and NaH (60% in mineral oil, 0.6 g, 12.4998 mmol, 2 eq) was added at 0° C. and stirred for 15 min. Mel (1.18 mL, 19.2306 mmol, 1.3 eq) was added dropwise to the RM at 0° C. The RM was brought to RT and stirred for 5 h. After completion of the reaction, the RM was quenched in cold water and extracted by ethyl acetate. The combined organic fractions were dried over Na₂SO₄, concentrated, and then purified by flash column chromatography (15% ethyl acetate in hexanes) to give compound (71_Int-1). MS(ES): 274 m/z [M+1]+, 1H NMR (400 MHz, DMSO-d6) δ 1.267 (t, J=7.2 Hz, 3H), 3.268 (s, 3H), 4.235-4.182 (m, 2H), 4.382 (s, 2H), 5.536 (s, 2H), 7.248 (t, J=8 Hz 4H), 7.877 (s, 1H), 8.468 (s, 1H).

### Step-2. Synthesis of N-(4-carbamimidoylbenzyl)-1-(4-(methoxymethyl)benzyl)-1H-pyrazole-4-carboxamide (71)

The final compound was prepared from ethyl 1-(4-(methoxymethyl)benzyl)-1H-pyrazole-4-carboxylate (71_Int-1) and 4-(aminomethyl)benzimidamide dihydrochloride in a similar fashion to that described in (Example-24)(71). MS(ES): 378.28 m/z [M+1]+, LCMS purity: 100%, HPLC purity: 98.18% 1H 400 MHz, DMSO-d6: δ 3.275 (s, 3H), 4.390 (s, 2H), 4.487 (d, J=6.0 Hz, 2H), 5.353 (s, 2H), 7.248-7.318 (m, 4H), 7.50 (d, J=8.0 Hz, 2H), 7.758 (d, J=8.0 Hz, 2H), 7.924 (s, 1H), 8.281 (s, 1H), 8.791 (t, J=6.0 Hz, 1H), 9.074 (s, 2H), 9.264 (s, 2H).

### Example 34: N-(4-carbamimidoylbenzyl)-1-(4-(cyanomethyl)benzyl)-5-cyclopropyl-1H-pyrazole-4-carboxamide (86)

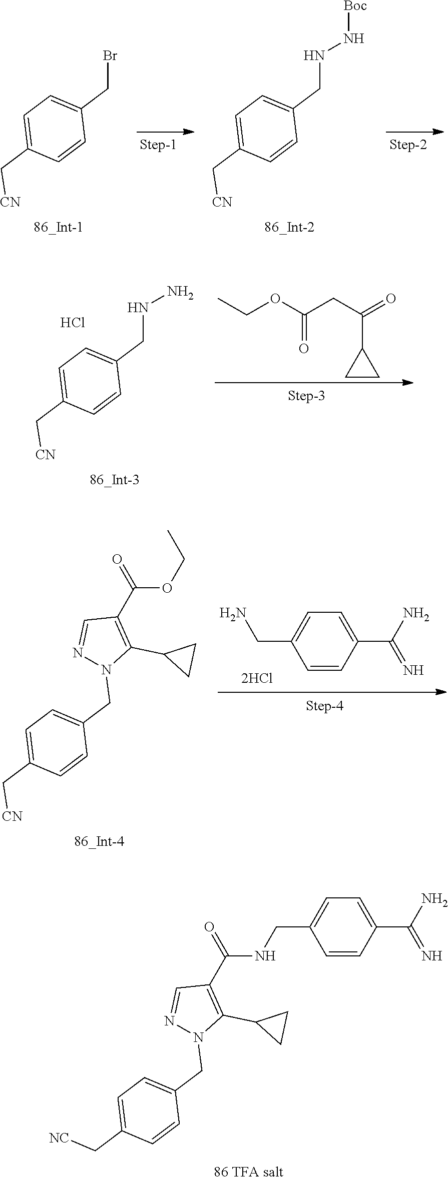

### Step-1. Synthesis of tert-butyl 2-(4-(cyanomethyl)benzyl) hydrazine-1-carboxylate (86_Int-2)

A stirred solution of 2-(4-(bromomethyl)phenyl) acetonitrile (86_Int-1)(3 g, 14.2857 mmol, 1.0 eq) in dimethylacetamide (30 mL, 10V) was prepared at RT. DIPEA (3.68 g, 28.5714 mmol, 2.0 eq) and t-butyl carbazate (3.77 g, 28.5714 mmol, 2.0 eq) were then added at RT. The RM was then heated to 70° C. and stirred for 16 h. After completion of the reaction, the RM was quenched in DM water and extracted by ethyl acetate. The combined organic fractions were washed by brine solution, dried over Na₂SO₄; concentrated and then purified by flash column chromatography (10-14% ethyl acetate in hexanes) to give compound (86_Int-2) MS(ES): 261 m/z [M+1]+, LCMS purity: 70%.

### Step-2. Synthesis of 2-(4-(hydrazinylmethyl)phenyl) acetonitrile (86_Int-3)

A stirred solution of tert-butyl 2-(4-(cyanomethyl)benzyl) hydrazine-1-carboxylate (86_Int-2)(1.0 g, 7.6628 mmol, 1.0 eq) was prepared in methanol (10 mL, 5V) and water (22 mL, 11V). Concentrated HCl (6.6 mL, 3.3V) was then added to the RM at RT. The RM was heated to 80° C. and stirred for 16 h. After completion of the reaction, the RM was evaporated and triturated in ethyl acetate to give compound (86_Int-3) MS(ES): 162.3 m/z [M+1]+, LCMS purity: 64%.

### Step-3. Synthesis of ethyl 1-(4-(cyanomethyl)benzyl)-5-cyclopropyl-1H-pyrazole-4-carboxylate (86_Int-4)

A solution of ethyl 3-cyclopropyl-3-oxopropanoate (1.0 g, 4.61531 mmol, 1.0 eq) and dimethylformamide dimethyl acetal (0.6 g, 4.2307 mmol, 1.1 eq) was prepared and stirred at 75° C. for 90 min. The RM was cooled to RT followed by addition of ethanol (12 mL, 20V), TEA (2.0 mL, 15.3844 mmol, 4.0 eq) and 2-(4-(hydrazinylmethyl)phenyl) acetonitrile (86_Int-3) (0.600 g, 3.8461 mmol, 1.0 eq). The RM was heated to 80° C. and stirred for 16 h. After completion of the reaction, the RM was quenched in water and extracted by ethyl acetate. The combined organic fractions were washed with a brine solution, dried over Na₂SO₄, concentrated, and then purified by flash column chromatography (12-15% ethyl acetate in hexanes) to give compound (86_Int-4). MS(ES): 310 m/z [M+1]+, LCMS purity: 64%, 1H 400 MHz, DMSO-d6: δ 0.754 (d, J=6.40 Hz, 2H), 0.963 (q, J=6.4 Hz, 2H), 1.790-1.756 (m, 1H), 4.106 (s, 2H), 4.205-4.152 (m, 2H), 5.459 (s, 2H), 7.166 (d, J=8 Hz, 2H), 7.240 (d, J=8 Hz 2H), 7.814 (s, 1H).

### Step-4. Synthesis of N-(4-carbamimidoylbenzyl)-1-(4-(cyanomethyl)benzyl)-5-cyclopropyl-1H-pyrazole-4-carboxamide (86)

The final compound was prepared from ethyl 1-(4-(cyanomethyl)benzyl)-5-cyclopropyl-1H-pyrazole-4-carboxylate (86_Int-4) and 4-(aminomethyl)benzimidamide dihydrochloride in a similar fashion to that described in (Example-24)(86). MS(ES): 412.5 m/z [M+1]+, LCMS purity: 100%, HPLC purity: 100% 1H 400 MHz, DMSO-d6: δ 0.754 (d, J=6.40 Hz, 2H), 0.963 (q, J=6.4 Hz, 2H), 1.790-1.756 (m, 1H), 4.025 (s, 2H), 4.487 (d, J=6.4 Hz, 2H), 5.440 (s, 2H), 7.196 (d, J=8 Hz, 2H), 7.33 (d, J=8 Hz, 2H), 7.522 (d, J=8 Hz 2H), 8.538 (d, J=6.4 Hz, 2H), 8.964 (s, 2H), 9.266 (s, 2 h).

### Example 35: N-(4-carbamimidoylbenzyl)-1-(4-(2-methoxyethyl)benzyl)-5-(methoxymethyl)-1H-pyrazole-4-carboxamide (87)

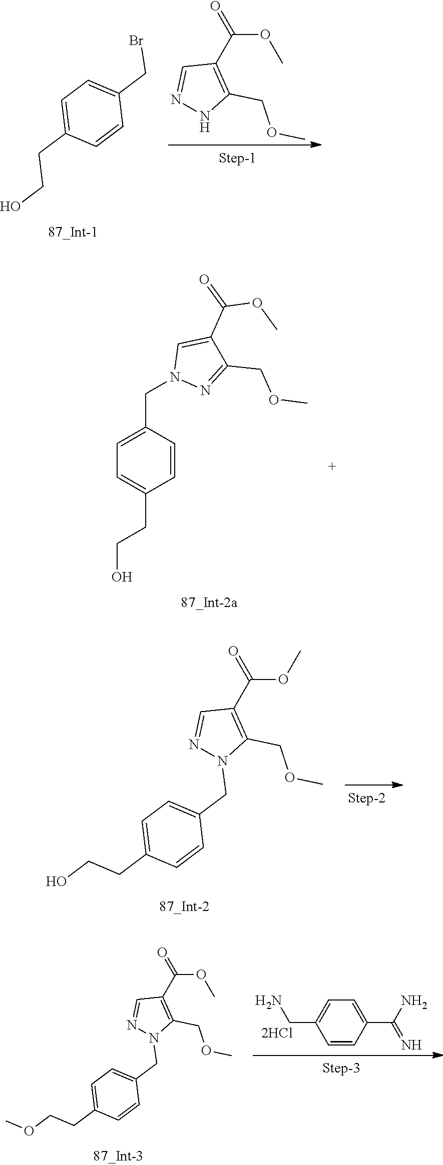

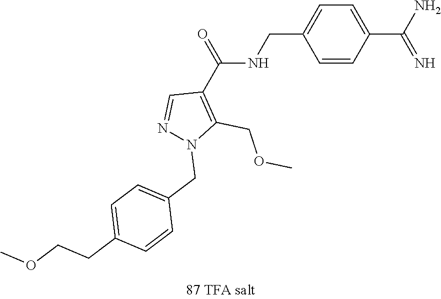 [SMILES: COCCc1ccc(Cn2ncc(C(=O)NCc3ccc(C(=N)N)cc3)c2COC)cc1]

### Step-1. Synthesis of methyl 1-(4-(2-hydroxyethyl)benzyl)-3-(methoxymethyl)-1H-pyrazole-4-carboxylate (87_Int-2)

A stirred solution of methyl 5-(methoxymethyl)-1H-pyrazole-4-carboxylate (3.2 g, 1.88235 mmol, 1.0 eq) in DMF (32 mL, 10V) was prepared and NaHMDS (1M in THF)(18.88 mL, 1.88235 mmol, 1.0 eq) was added at 0° C. and stirred for 30 min. 2-(4-(bromomethyl)phenyl) ethan-1-ol (87_Int-1)(4.0 g, 1.88235 mmol, 1.0 eq) was added to the RM at 0° C. The RM was brought to RT and stirred for 16 h. After completion of the reaction, the RM was quenched in water and extracted by ethyl acetate. The combined organic fractions were dried over Na₂SO₄, concentrated, and then purified by flash column chromatography (5% ethyl acetate in hexanes)(87_Int-2). Confirmed by 2D NMR (ROE analysis). MS(ES): 304.9 m/z [M+1]+, LCMS purity: 99.72%, 1H 400 MHz, DMSO-d6: δ 2.679 (t, J=4 Hz, 3H), 3.242 (s, 3H), 3.555 (t, J=4 Hz, 2H), 3.724 (s, 3H), 4.490 (s, 2H), 5.276 (s, 2H), 7.203-7.184 (m, 4H), 8.416 (s, 1H).

### Step-2. Synthesis of methyl 1-(4-(2-methoxyethyl)benzyl)-5-(methoxymethyl)-1H-pyrazole-4-carboxylate (87_Int-3)

A stirred solution of methyl 1-(4-(2-hydroxyethyl)benzyl)-5-(methoxymethyl)-1H-pyrazole-4-carboxylate (87_Int-2)(0.4 g, 1.3157 mmol, 1.0 eq) in DMF (4 ml, 10V) was prepared. NaH (60% in mineral oil)(0.126 g, 2.6314 mmol, 2 eq) was added slowly and the RM was stirred 15 min at 0° C., followed by the dropwise addition of. MeI (0.106 mL, 1.7106 mmol, 1.3 eq). The RM was brought to RT and stirred for 5 h. After completion of the reaction, the RM was quenched in cold water and extracted by ethyl acetate. The combined organic fractions were dried over Na₂SO₄, concentrated, and then purified by flash column chromatography (15% ethyl acetate in hexanes) to give compound (87_Int-3). MS(ES): 319 m/z [M+1]+, LCMS purity: 79%, ¹H NMR (400 MHz, DMSO-d6) δ 400 MHz, DMSO-d6: δ 2.489 (t, J=6.40 Hz, 2H), 3.211 (s, 3H), 3.225 (s, 3H), 3.518 (t, J=6.4 Hz, 2H), 4.794 (s, 2H), 5.358 (s, 2H), 7.111 (d, J=8 Hz, 2H), 7.200 (d, J=8 Hz, 2H), 7.905 (s, 2H).

### Step-3. N-(4-carbamimidoylbenzyl)-1-(4-(2-methoxyethyl)benzyl)-5-(methoxymethyl)-1H-pyrazole-4-carboxamide (87)

The final compound was prepared from methyl 1-(4-(2-methoxyethyl)benzyl)-5-(methoxymethyl)-1H-pyrazole-4-carboxylate (87_Int-3) and 4-(aminomethyl)benzimidamide dihydrochloride in a similar fashion to that described in (Example-24)(87). MS(ES): 435.53 m/z [M+1]+, LCMS purity: 100%, HPLC purity: 100% 1H 400 MHz, DMSO-d6: δ 2.489 (t, J=6.40 Hz, 2H), 3.211 (s, 3H), 3.225 (s, 3H), 3.503 (t, J=6.4 Hz, 2H), 4.492 (d, J=6 Hz, 2H), 4.802 (d, 2H), 5.310 (d, 2H), 7.111 (d, J=8 Hz, 2H), 7.183 (d, J=8 Hz, 2H), 7.508 (d, J=8 Hz, 2H), 7.752 (d, J=8 Hz, 2H). 8.012 (s, 1H), 8.817 (t, J=6 Hz, 1H), 8.911 (s, 2H), 9.252 (s, 2H).

### Example 36: Preparation of N-(4-carbamimidoylbenzyl)-1-(4-(2-(dimethylamino)-2-oxoethyl)benzyl)-1H-pyrazole-4-carboxamide (33)

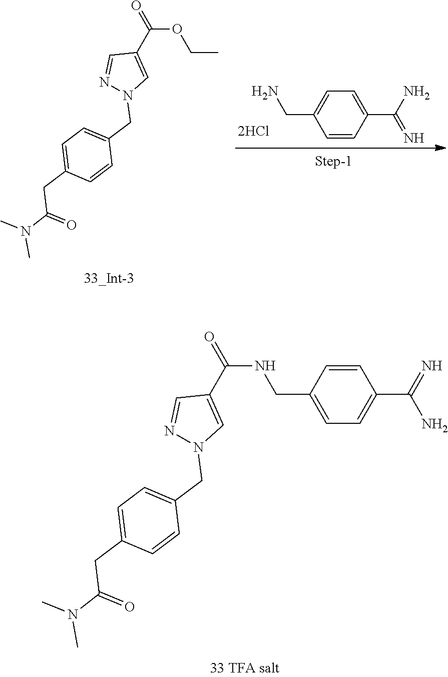 [SMILES: CCOC(=O)c1cnn(Cc2ccc(CC(=O)N(C)C)cc2)c1 ; N=C(N)c1ccc(CN)cc1 ; CN(C)C(=O)Cc1ccc(Cn2cc(C(=O)NCc3ccc(C(=N)N)cc3)cn2)cc1]

### Step-1. Synthesis of N-(4-carbamimidoylbenzyl)-1-(4-(2-(dimethylamino)-2-oxoethyl)benzyl)-1H-pyrazole-4-carboxamide (33)

The final compound was prepared from ethyl 1-(4-(2-(dimethylamino)-2-oxoethyl)benzyl)-1H-pyrazole-4-carboxylate (33_Int-3) and 4-(aminomethyl)benzimidamide dihydrochloride in a similar fashion to that described in (Example-24)(33). MS(ES): 419.4 m/z [M+H]+, LCMS purity: 100%, HPLC purity: 95.56% ¹H NMR (400 MHz, DMSO-d6) § 2.807 (s, 3H), 2.978 (s, 3H), 3.656 (s, 2H), 4.778 (d, J=6.0 Hz, 2H), 7.183-7.197 (m, 5H), 7.483-7.504 (m, 2H), 7.734-7.755 (m, 1H), 7.837 (s, 1H), 7.907 (s, 1H), 8.269 (s, 1H), 8.767 (t, J=6.0 Hz, 1H), 8.946 (s, 2H), 9.245 (s, 2H).

### Example 37: Preparation of N-(4-carbamimidoylbenzyl)-1-(2-(cyanomethyl)benzyl)-1H-pyrazole-4-carboxamide (70)

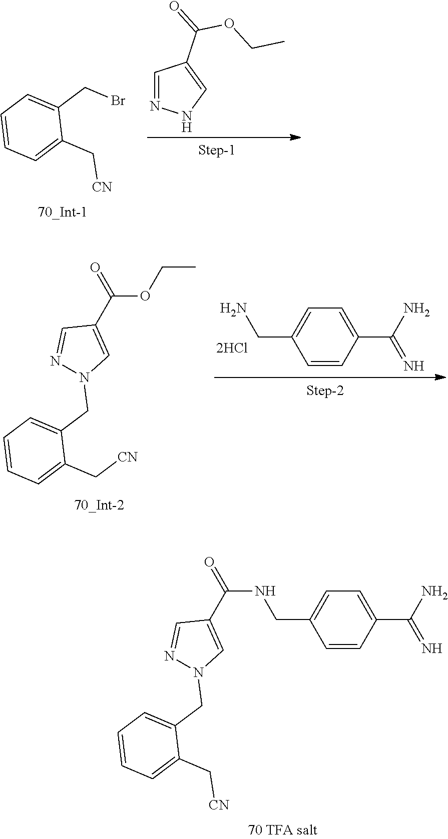

### Step-1. Synthesis of ethyl 1-(2-(cyanomethyl)benzyl)-1H-pyrazole-4-carboxylate (70_Int-2)

A stirred solution of ethyl 1H-pyrazole-4-carboxylate (0.1 g, 0.71 mmol, 1.0 eq) was prepared in DMF (2 mL, 20V) and 2-(2-(bromomethyl)phenyl) acetonitrile (70_Int-1)(0.149 g, 0.71 mmol, 1.0 eq) was added at RT. The RM was then heated to 80° C. and stirred for 16 h. After completion of the reaction, the RM was quenched in water and extracted by ethyl acetate. The combined organic fractions were dried over Na₂SO₄, concentrated, and purified by flash column chromatography (25% ethyl acetate in hexanes) to give compound MS(ES): 270.m/z [M+1]+, LCMS purity: 98.57%, 1H 400 MHz, DMSO-d6: δ 1.244 (t, J=6.4 Hz, 3H), 4.225-4.172 (m, 4H), 5.448 (s, 2H), 7.119 (d, J=7.20 Hz, 1H), 7.454-7.332 (m, 3H), 7.892 (s, 1H), 8.453 (s, 1H).

### Step-2. Synthesis of N-(4-carbamimidoylbenzyl)-1-(2-(cyanomethyl)benzyl)-1H-pyrazole-4-carboxamide (70)

The final compound was prepared from ethyl 1-(2-(cyanomethyl)benzyl)-1H-pyrazole-4-carboxylate (70_Int-2) and 4-(aminomethyl)benzimidamide dihydrochloride in a similar fashion to that described in (Example-24)(70). MS(ES): 373.45 m/z [M+H]+, LCMS purity: 100%, HPLC purity: 98.64. ¹H 400 MHz, DMSO-d6: δ 4.185 (s, 2H), 4.483 (d, J=6.0 Hz, 2H), 5.444 (s, 2H), 7.119 (s, 1H), 7.345-7.396 (m, 2H), 7.448-7.504 (m, 3H), 7.746 (d, J=8.0 Hz, 2H), 7.943 (s, 1H), 8.259 (s, 1H), 8.784 (t, J=6.0 Hz, 1H), 8.952 (s, 2H), 9.247 (s, 2H).

### Example 38: Preparation of N-(4-carbamimidoyl-3-fluorobenzyl)-1-(4-(2-(methylamino)-2-oxoethyl)benzyl)-1H-pyrazole-4-carboxamide (74)

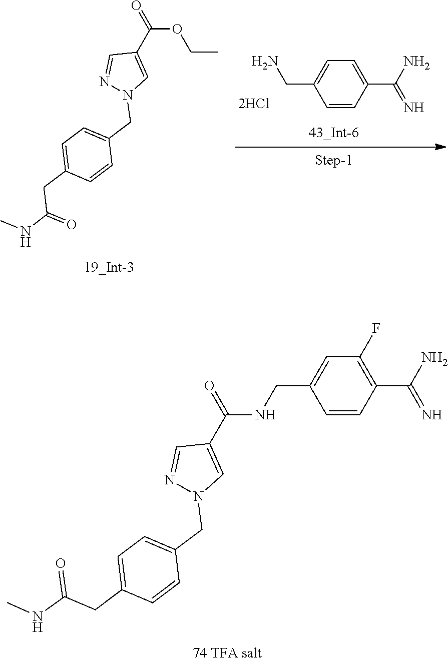 [SMILES: CCOC(=O)c1cnn(Cc2ccc(CC(=O)NC)cc2)c1 ; N=C(N)c1ccc(CN)cc1 ; CNC(=O)Cc1ccc(Cn2cc(C(=O)NCc3ccc(C(=N)N)c(F)c3)cn2)cc1]

### Step-1. Synthesis of N-(4-carbamimidoyl-3-fluorobenzyl)-1-(4-(2-(methylamino)-2-oxoethyl)benzyl)-1H-pyrazole-4-carboxamide (74)

The final compound was prepared from ethyl 1-(4-(2-(methylamino)-2-oxoethyl)benzyl)-1H-pyrazole-4-carboxylate (19_Int-3) and 4-(aminomethyl)-2-fluorobenzimidamide dihydrochloride (43_Int-6) in a similar fashion to that described in (Example-24)(74). MS(ES): 423.4 m/z [M+H]+, LCMS purity: 100%, HPLC purity: 98.51% ¹H NMR (400 MHz, DMSO-d6) δ 2.678 (s, 3H), 3.367 (s, 2H), 4.467 (d, J=6 Hz, 2H), 5.317 (s, 2H), 7.196-7.243 (m, 4H), 7.328 (t, J=8.4 Hz, 2H), 7.262 (t, J=8.4 Hz, 1H), 7.906 (s, 1H), 7.961 (s, 1H), 8.268 (s, 1H), 8.807 (t, J=6.0 Hz, 1H), 9.186 (s, 2H), 9.387 (s, 2H).

### Example 39: Preparation N-(4-carbamimidoylbenzyl)-1-(4-(2-cyanopropan-2-yl)benzyl)-5-(methoxymethyl)-1H-pyrazole-4-carboxamide (82)

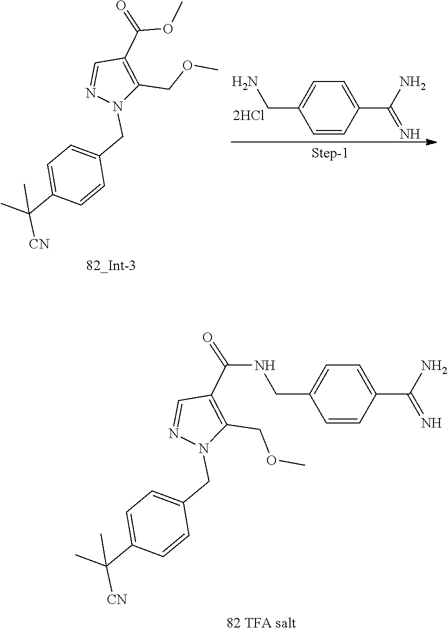

### Step-1. Synthesis of N-(4-carbamimidoylbenzyl)-1-(4-(2-cyanopropan-2-yl)benzyl)-5-(methoxymethyl)-1H-pyrazole-4-carboxamide (82)

The final compound was prepared from methyl 1-(4-(2-cyanopropan-2-yl)benzyl)-5-(methoxymethyl)-1H-pyrazole-4-carboxylate (82_Int-3) and 4-(aminomethyl)benzimidamide dihydrochloride in a similar fashion to that described in (Example-24)(70). MS(ES): 445.4 m/z [M+H]+, LCMS purity: 100%, HPLC purity: 100% ¹H NMR (400 MHz, DMSO-d6) δ 1.675 (s, 6H), 3.246 (s, 3H), 4.514 (d, J=6 Hz, 2H), 4.480 (s, 2H), 5.380 (s, 2H), 7.274 (d, J=8.4 Hz, 2H), 7.486-7.537 (m, 4H), 7.777 (d, J=8.4 Hz, 2H), 8.046 (s, 1H), 8.843 (t, J=6 Hz, 1H), 9.022 (s, 2H), 9.274 (s, 2H).

### Example 40: Preparation N-(4-carbamimidoylbenzyl)-1-(4-(2-cyanopropan-2-yl)benzyl)-5-(methoxymethyl)-1H-pyrazole-4-carboxamide (85)

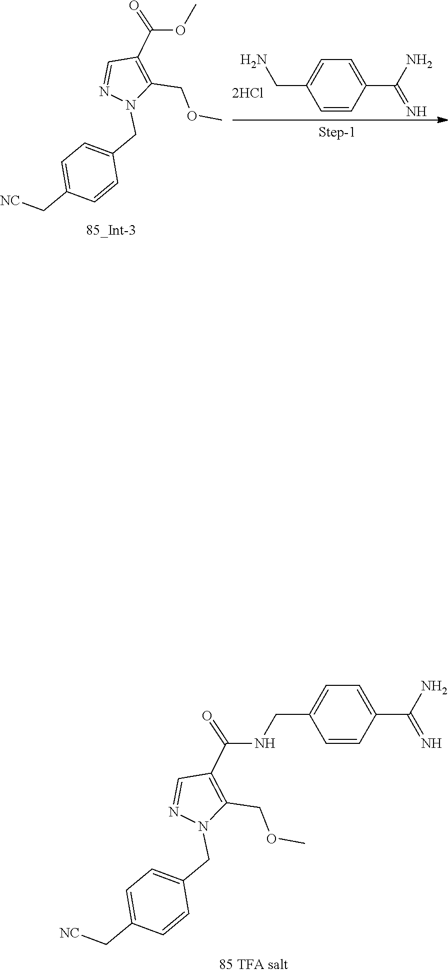 [SMILES: COCc1c(C(=O)OC)cnn1Cc1ccc(CC#N)cc1 ; N=C(N)c1ccc(CN)cc1 ; COCc1c(C(=O)NCc2ccc(C(=N)N)cc2)cnn1Cc1ccc(CC#N)cc1]

### Step-1. Synthesis of N-(4-carbamimidoylbenzyl)-1-(4-(cyanomethyl)benzyl)-5-(methoxymethyl)-1H-pyrazole-4-carboxamide (85)

The final compound was prepared from methyl 1-(4-(cyanomethyl)benzyl)-5-(methoxymethyl)-1H-pyrazole-4-carboxylate (85_Int-3) and 4-(aminomethyl)benzimidamide dihydrochloride in a similar fashion to that described in (Example-24)(70). MS(ES): 417.4 m/z [M+H]+, LCMS purity: 100%, HPLC purity: 100% ¹H NMR (400 MHz, DMSO-d6) δ 3.233 (s, 3H), 4.029 (s, 2H), 4.514 (d, J=6 Hz, 2H), 4.829 (s, 2H), 5.375 (s, 2H), 7.232 (d, J=8 Hz, 2H), 7.327 (d, J=8 Hz, 2H), 7.529 (d, J=8.0 Hz, 2H), 7.774 (d, J=8.0 Hz, 2H), 8.046 (s, 1H), 8.839 (t, J=6 Hz, 1H), 8.963 (s, 2H), 9.271 (s, 2H).

### Example 41: Preparation of N-(4-carbamimidoyl-3-fluorobenzyl)-1-(4-(2-(dimethylamino)-2-oxoethyl)benzyl)-1H-pyrazole-4-carboxamide (78)

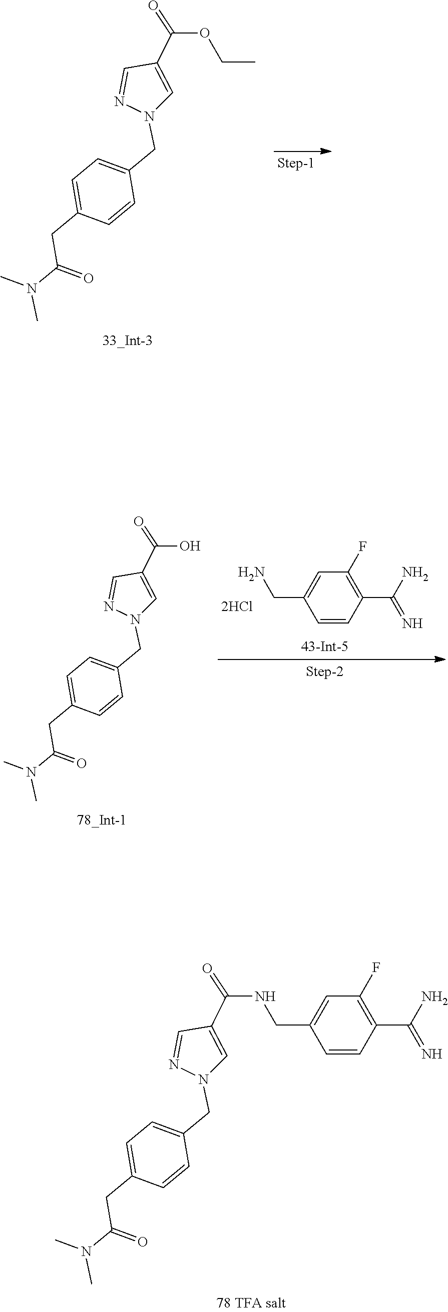 [SMILES: CCOC(=O)c1cnn(Cc2ccc(CC(=O)N(C)C)cc2)c1 ; CN(C)C(=O)Cc1ccc(Cn2cc(C(=O)O)cn2)cc1 ; N=C(N)c1ccc(CN)cc1F ; CN(C)C(=O)Cc1ccc(Cn2cc(C(=O)NCc3ccc(C(=N)N)c(F)c3)cn2)cc1]

### Step-1. Synthesis of 1-(4-(2-(dimethylamino)-2-oxoethyl)benzyl)-1H-pyrazole-4-carboxylic acid (78_Int-1)

A stirred solution of ethyl 1-(4-(2-(dimethylamino)-2-oxoethyl)benzyl)-1H-pyrazole-4-carboxylate (33_Int-3)(0.5 g, 0.158 mmol, 1.0 eq) in water:methanol:THF (1:1:1)(5 mL, 10V) was prepared and LiOH (0.3 g, 0.793 mmol, 5 eq) was added at RT and stirred for 16 h. After completion of the reaction, the RM was quenched in 2N HCl (approximately pH 4) and extracted with 10% methanol in DCM. The combined organic fractions were dried over Na₂SO₄; concentrated to give compound (78_Int-1) MS(ES): 288.4.m/z [M+H]+, LCMS purity: 97.47%, ¹H NMR (400 MHz, DMSO-d6) δ 2.804 (s, 3H), 2.975 (s, 3H), 5.315 (s, 2H), 7.191-7.219 (m, 4H), 7.795 (s, 1H), 8.357 (s, 1H), 12.301 (s, 1H).

### Step-2. Synthesis of N-(4-carbamimidoyl-3-fluorobenzyl)-1-(4-(2-(dimethylamino)-2-oxoethyl)benzyl)-1H-pyrazole-4-carboxamide (78)

A stirred solution of 1-(4-(2-(dimethylamino)-2-oxoethyl)benzyl)-1H-pyrazole-4-carboxylic acid (78_Int-1)(0.2 g, 0.069 mmol, 1.0 eq) in pyridine (2 mL, 10V) was prepared. -(aminomethyl)-2-fluorobenzimidamide dihydrochloride (43_Int-5)(0.2 g, 0.083 mmol, 1.2 eq) and EDC HCl (0.67 g, 0.348 mmol, 5.0 eq) were added to the RM at RT and stirred for 16 h. After completion of the reaction, the RM was concentrated and purified by PREP HPLC ((A)_{0.1}% TFA in water (B)₁₀₀% MeCN). Pure fractions were lyophilized to give compound (78) MS(ES): 437.36.m/z [M+H]+, LCMS purity: 98.37%, HPLC purity: 99.70% ¹H NMR (400 MHz, DMSO-d6) δ 2.804 (s, 3H), 2.981 (s, 3H), 3.653 (s, 2H), 4.459 (d, J=6.4 Hz, 2H), 5.314 (s, 2H), 7.194 (s, 4H), 7.318 (t, J=11.6 Hz, 2H), 7.615 (t, J=11.6 Hz, 1H), 7.900 (s, 1H), 8.266 (s, 1H), 8.790 (t, J=6 Hz, 1H), 9.170 (s, 2H), 9.370 (s, 2H).

### Biological Examples

Plasma kallikrein protease inhibition assay. Two methods for determining the IC₅₀ of a test compound against Plasma Kallikrein are provided.

In the first method was used a reaction buffer composed of 25 mM Tris-HCl (pH 8.0), 100 mM NaCl (pH 8.5), 0.01% Brij35, and 1% DMSO (final). The enzyme used was Plasma Kallikrein (R&D Systems Cat #2497-SE; Recombinant Human Plasma Kallikrein, expressed in Mouse myeloma cell line, NS0-derived Gly20-Ala638, with a C-terminal 60-His tag. MW=70 kDa).

The enzyme was activated by dilution to 200 μg/mL in activation buffer (100 mM Tris, 10 mM CaCl2, 150 mM NaCl, pH 7.5 (TCN), and then combined with an equal volume of 20 μg/mL thermolysin to form a reaction buffer. Each test compound was then dissolved in DMSO and delivered into the reaction buffer. The reaction was initiated by delivering a substrate solution containing 10 μM Z-FR-AMC (Enzo Cat #P-139; AMC: 7-Amino-4-methylcoumarin) into the reaction well after a 20-minute pre-incubation period.

Measurement was conducted with En Vision (PE) with excitation and emission wavelengths of 355 nm and 460 nm, respectively. The reaction was stopped with EDTA. The enzyme activities were monitored every 5 minutes as a time-course measurement of the increase in signal from fluorescently labeled peptide substrate for 120 minutes at room temperature.

Data was analyzed by taking slope*(signal/time) of the linear portion of measurements. Slope was calculated using Excel, and curve fits were performed using Prism software.

Second, Plasma Kallikrein activity was also measured in pooled human plasma. First, a 10% Actin FS solution was prepared in assay buffer. Each test compound was dissolved in DMSO and delivered to the reaction mixture along with Z-FR-AMC substrate and pooled human plasma. The multi-well reaction plate was incubated for five minutes at room temperature. To initiate the reaction, the 10% Actin FS solution was added to each well, and kinetic measurements were taken at Ex/Em at 355/460 nm. The fluorescence signal was recorded every 30 seconds for a total of 10 minutes.

Permeability Assay. A permeability study was conducted using Caco-2 cells (ECACC Cat. no. 09042001) seeded on cell inserts plate (Millicell, Cat #PSHT010R⁵) at a density of 80,000 cells per well, and the cells were maintained for 18-21 days in culture medium (1×DMEM with 10% FBS, 0.1 mg/mL Penicillin/Streptomycin). The culture medium was changed every alternate day. Prior to the experiments, the integrity of the cellular monolayer was evaluated by measuring the TEER value using a volt ohm meter and STX100C₉₆ electrode. Only monolayers with TEER values greater than 800 ohm·cm² in buffer after the first wash were used.

An assay buffer (HBSS with Ca+2 and Mg+2 buffered with 10 mM HEPES and 25 mM D-Glucose, pH-7.4) was used on apical side as well as on basolateral side.

An intermediate stock solution of a test compound was prepared in DMSO at a concentration of 1 mM in DMSO. This stock solution was spiked in the assay buffer to achieve a target test compound concentration of 10 μM. The organic content of final drug preparation was 1.0% v/v. The bidirectional permeability experiment was done in singlet and the sample analysis was done in duplicate.

The cultured cell monolayer was washed twice with assay buffer (0.4 mL and 0.8 mL was added to the apical and basolateral sides, respectively, of the culture plate), and then buffers from both compartments were discarded.

For the apical to basal (AP>BL) experiment, aliquots of 0.4 mL donor solution (assay buffer, pH 7.4 containing test compound) and 0.8 mL of receiver solutions (assay buffer, pH 7.4, only) were added to the apical and basolateral compartments, respectively. For the basal to apical (BL>AP) experiment, aliquots of 0.8 mL donor solution (assay buffer pH-7.4 containing test compound) and 0.4 mL of receiver solutions (assay buffer, pH-7.4) were added to the basolateral and apical compartments, respectively. The plate was then kept in an incubator at 37° C. for 120 minutes.

Control experiments with Propranolol (High Permeability), Atenolol (Low Permeability), Digoxin (High Efflux-Pgp substrate), and Digoxin+Verapamil (Pgp Inhibitor) in both directions (AP>BL and BL>AP) were performed in separate wells on the same experiment day.

After the completion of the transport experiment, the integrity of the cell monolayer was evaluated by measuring the Lucifer Yellow (LY) rejection. To do this, 400 μL of 10 μM LY was added to each well of the filter plate and incubated for 1 hour at 37° C. Subsequently, the samples were collected from the basolateral compartments, and the LY fluorescence was measured using an excitation wavelength of 485 nm and an emission wavelength of 530 nm. The percent LY rejection across the cell monolayer was calculated by measuring fluorescence in the receiver plate (basolateral compartment) compared to theoretical equilibrium standard.

The study samples (collected from apical and basolateral compartments after 120 min incubation) were analyzed by LCMS/MS, following which the Papp of the compound(s) was calculated in both apical to basal and basal to apical directions. Papp=([DBL]×VBL)/(A×t×[DAP]) where: [DBL]=final drug concentration on the basolateral side, VBL=volume of the basolateral compartment, A=surface area of the cell culture, t=total incubation time, and [DAP]=initial drug concentration on the apical side.

Results from the assays described above for representative compounds of the present disclosure are presented in Table 1 below. Scores for selected compounds in each assay are presented as follows:

| Purified Human | Pooled Human | Caco-2 Permeation |
|---|---|---|
| Plasma Assay (nM) | Plasma Assay (nM) | Assay (10⁻⁶ cm/s) |
| A < 1 | A < 100 | A < 2 |
| 1 ≤ B < 10 | 100 ≤ B < 1000 | 2 ≤ B < 10 |
| 10 ≤ C < 100 | 1000 < C | 10 < C |
| 100 < D | | |

TABLE 1
Plasma Kallikrein Inhibition and Cell Permeation of
Representative Compounds

| | | | Pooled | |
|---|---|---|---|---|
| | | | Human | Caco- |
| | | Purified | Plasma | 2 |
| | | PKal | PKal | Perm |
| | | IC50 | IC50 | (10^{31 6} |
| Example | Structure | (nM) | (nM) | cm/s) |
| 1 | 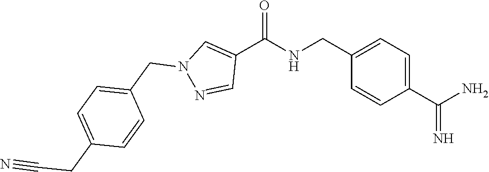 [SMILES: N#CCc1ccc(Cn2cc(C(=O)NCc3ccc(C(=N)N)cc3)cn2)cc1] | A | A | A |
| 2 | 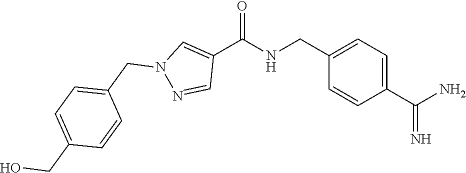 [SMILES: N=C(N)c1ccc(CNC(=O)c2cnn(Cc3ccc(CO)cc3)c2)cc1] | B | A | |
| 3 | 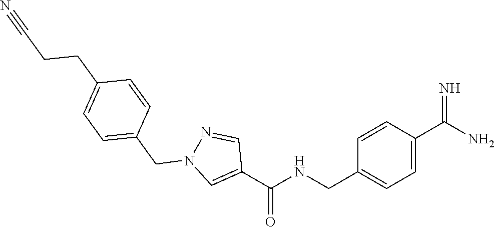 [SMILES: N#CCCc1ccc(Cn2cc(C(=O)NCc3ccc(C(=N)N)cc3)cn2)cc1] | A | A | A |
| 4 | 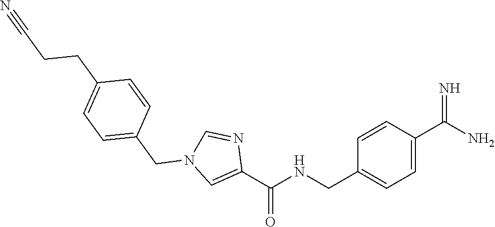 [SMILES: N#CCCc1ccc(Cn2cnc(C(=O)NCc3ccc(C(=N)N)cc3)c2)cc1] | A | | A |
| 5 | 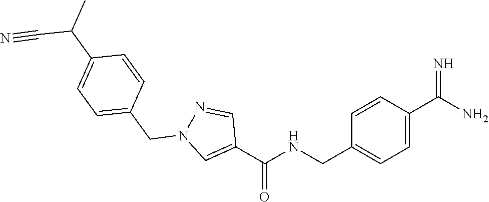 [SMILES: CC(C#N)c1ccc(Cn2cc(C(=O)NCc3ccc(C(=N)N)cc3)cn2)cc1] | B | | |
| 6 | 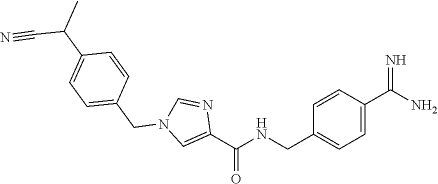 [SMILES: CC(C#N)c1ccc(Cn2cnc(C(=O)NCc3ccc(C(=N)N)cc3)c2)cc1] | | | |
| 7 | 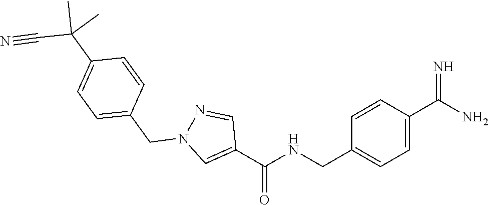 [SMILES: CC(C)(C#N)c1ccc(Cn2cc(C(=O)NCc3ccc(C(=N)N)cc3)cn2)cc1] | A | A | A |
| 8 | 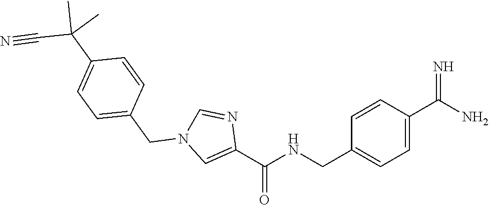 [SMILES: CC(C)(C#N)c1ccc(Cn2cnc(C(=O)NCc3ccc(C(=N)N)cc3)c2)cc1] | B | | A |
| 9 | 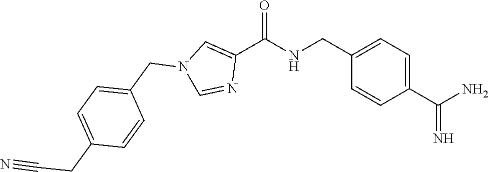 [SMILES: N#CCc1ccc(Cn2cnc(C(=O)NCc3ccc(C(=N)N)cc3)c2)cc1] | B | | B |
| 10 | 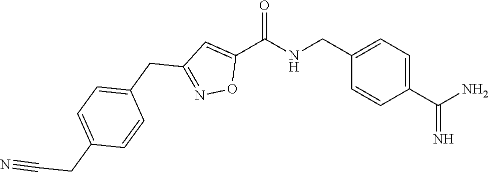 [SMILES: N#CCc1ccc(Cc2cc(C(=O)NCc3ccc(C(=N)N)cc3)on2)cc1] | B | | |
| 11 | 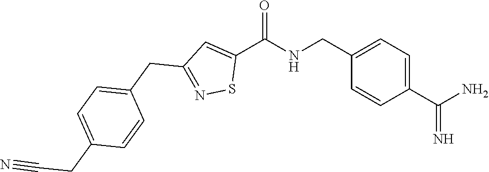 [SMILES: N#CCc1ccc(Cc2cc(C(=O)NCc3ccc(C(=N)N)cc3)sn2)cc1] | B | | |
| 12 | 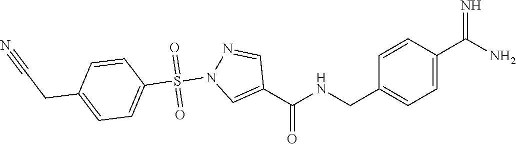 [SMILES: N#CCc1ccc(S(=O)(=O)n2cc(C(=O)NCc3ccc(C(=N)N)cc3)cn2)cc1] | C | B | |
| 13 | 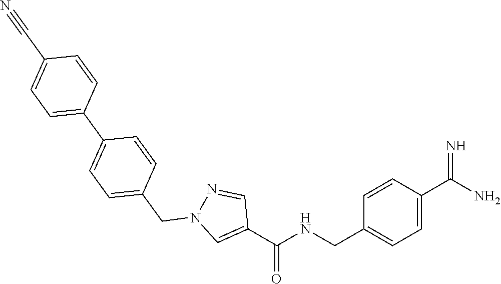 [SMILES: N#Cc1ccc(-c2ccc(Cn3cc(C(=O)NCc4ccc(C(=N)N)cc4)cn3)cc2)cc1] | A | A | |
| 14 | 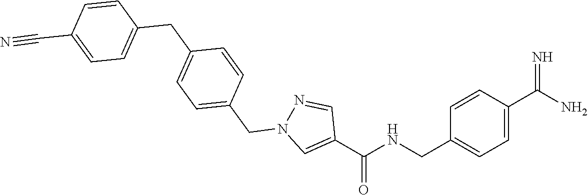 [SMILES: N#Cc1ccc(Cc2ccc(Cn3cc(C(=O)NCc4ccc(C(=N)N)cc4)cn3)cc2)cc1] | A | B | |
| 15 | 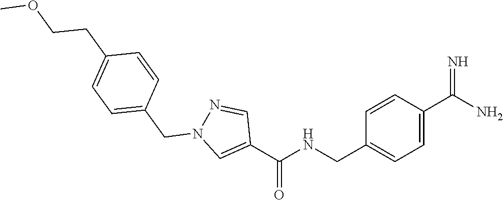 [SMILES: COCCc1ccc(Cn2cc(C(=O)NCc3ccc(C(=N)N)cc3)cn2)cc1] | A | A | B |
| 16 | 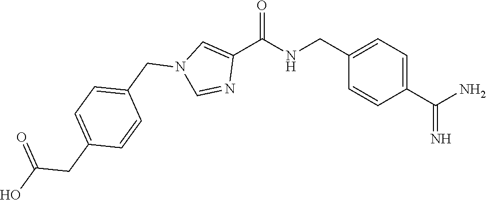 [SMILES: N=C(N)c1ccc(CNC(=O)c2cn(Cc3ccc(CC(=O)O)cc3)cn2)cc1] | D | | |
| 17 | 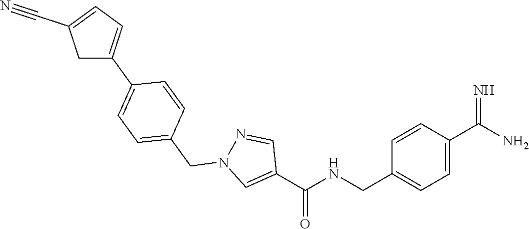 [SMILES: N#CC1=CC=C(c2ccc(Cn3cc(C(=O)NCc4ccc(C(=N)N)cc4)cn3)cc2)C1] | | | |
| 18 | 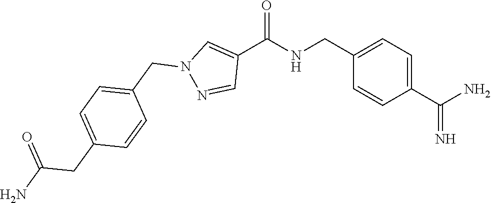 [SMILES: N=C(N)c1ccc(CNC(=O)c2cnn(Cc3ccc(CC(N)=O)cc3)c2)cc1] | B | | A |
| 19 | 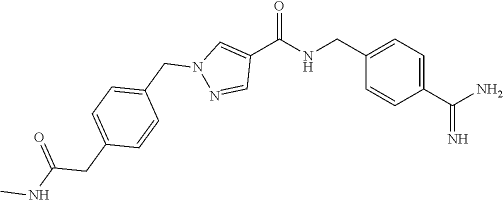 [SMILES: CNC(=O)Cc1ccc(Cn2cc(C(=O)NCc3ccc(C(=N)N)cc3)cn2)cc1] | A | A | A |
| 20 | 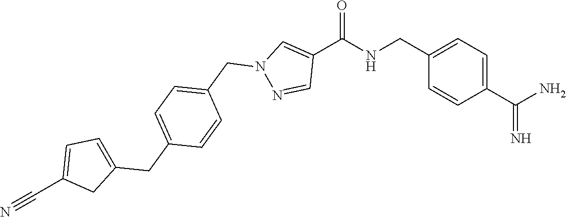 [SMILES: N#CC1=CC=C(Cc2ccc(Cn3cc(C(=O)NCc4ccc(C(=N)N)cc4)cn3)cc2)C1] | | | |
| 21 | 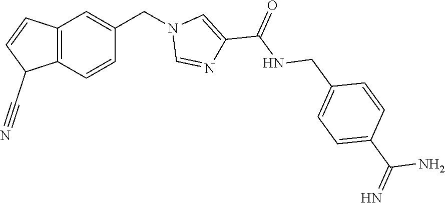 [SMILES: N#CC1C=Cc2cc(Cn3cnc(C(=O)NCc4ccc(C(=N)N)cc4)c3)ccc21] | | | |
| 22 | 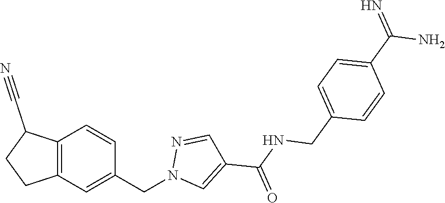 [SMILES: N#CC1CCc2cc(Cn3cc(C(=O)NCc4ccc(C(=N)N)cc4)cn3)ccc21] | | | |
| 23 | 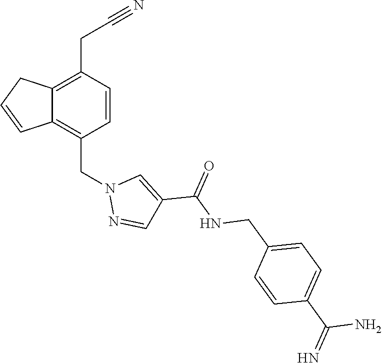 [SMILES: N#CCc1ccc(Cn2cc(C(=O)NCc3ccc(C(=N)N)cc3)cn2)c2c1CC=C2] | | | |
| 24 | 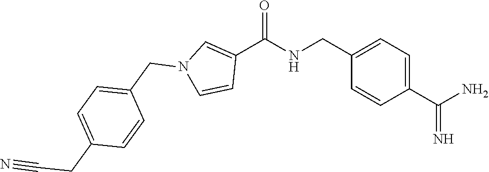 [SMILES: N#CCc1ccc(Cn2ccc(C(=O)NCc3ccc(C(=N)N)cc3)c2)cc1] | | | |
| 25 | 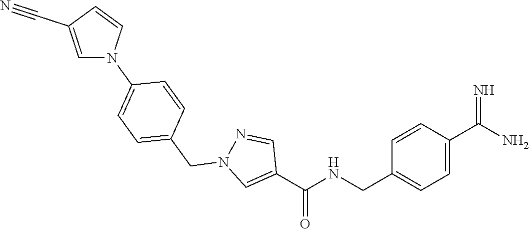 [SMILES: N#Cc1ccn(-c2ccc(Cn3cc(C(=O)NCc4ccc(C(=N)N)cc4)cn3)cc2)c1] | A | A | |
| 26 | 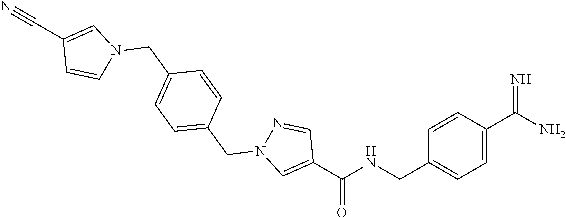 [SMILES: N#Cc1ccn(Cc2ccc(Cn3cc(C(=O)NCc4ccc(C(=N)N)cc4)cn3)cc2)c1] | A | A | A |
| 27 | 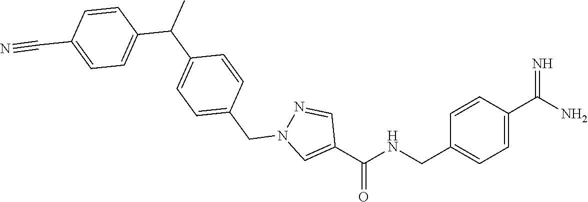 [SMILES: CC(c1ccc(C#N)cc1)c1ccc(Cn2cc(C(=O)NCc3ccc(C(=N)N)cc3)cn2)cc1] | | | |
| 28 | 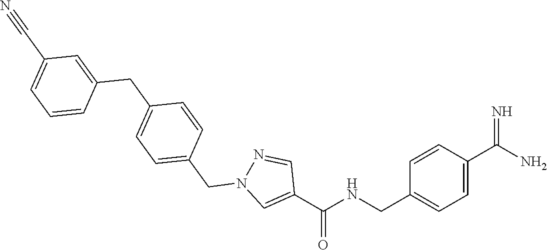 [SMILES: N#Cc1cccc(Cc2ccc(Cn3cc(C(=O)NCc4ccc(C(=N)N)cc4)cn3)cc2)c1] | A | A | A |
| 29 | 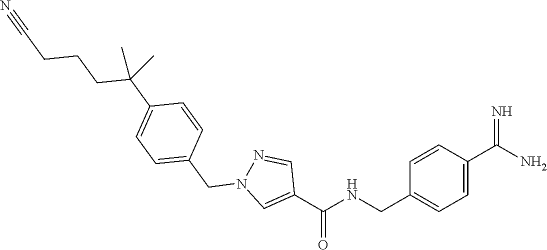 [SMILES: CC(C)(CCCC#N)c1ccc(Cn2cc(C(=O)NCc3ccc(C(=N)N)cc3)cn2)cc1] | | | |
| 30 | 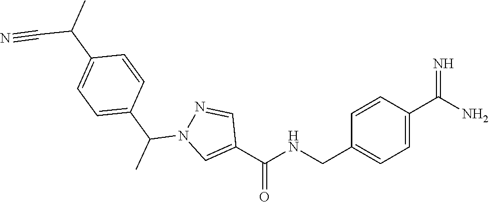 [SMILES: CC(C#N)c1ccc(C(C)n2cc(C(=O)NCc3ccc(C(=N)N)cc3)cn2)cc1] | | | |
| 31 | 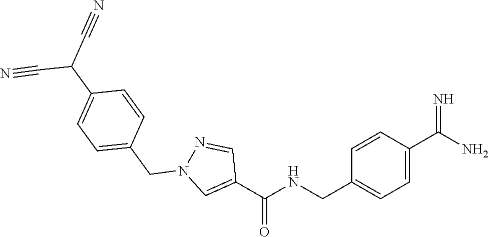 [SMILES: N#CC(C#N)c1ccc(Cn2cc(C(=O)NCc3ccc(C(=N)N)cc3)cn2)cc1] | | | |
| 32 | 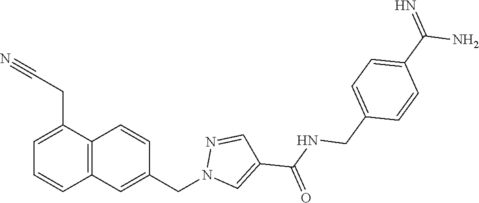 [SMILES: N#CCc1cccc2cc(Cn3cc(C(=O)NCc4ccc(C(=N)N)cc4)cn3)ccc12] | | | |
| 33 | 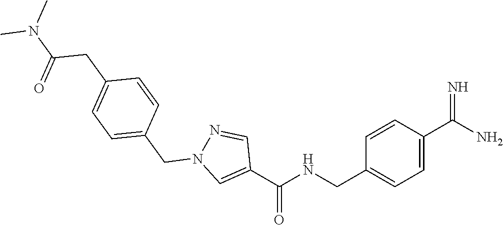 [SMILES: CN(C)C(=O)Cc1ccc(Cn2cc(C(=O)NCc3ccc(C(=N)N)cc3)cn2)cc1] | A | A | A |
| 34 | 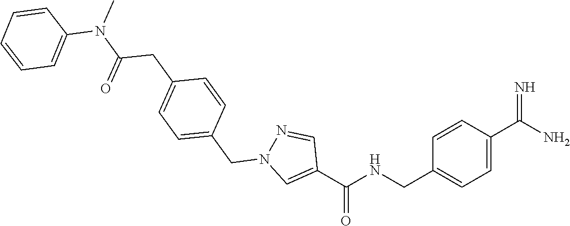 [SMILES: CN(C(=O)Cc1ccc(Cn2cc(C(=O)NCc3ccc(C(=N)N)cc3)cn2)cc1)c1ccccc1] | | | |
| 35 | 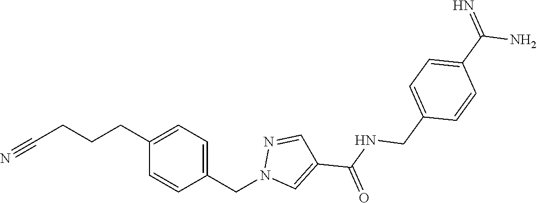 [SMILES: N#CCCCc1ccc(Cn2cc(C(=O)NCc3ccc(C(=N)N)cc3)cn2)cc1] | | | |
| 36 | 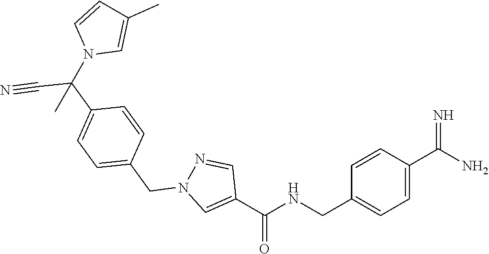 [SMILES: Cc1ccn(C(C)(C#N)c2ccc(Cn3cc(C(=O)NCc4ccc(C(=N)N)cc4)cn3)cc2)c1] | | | |
| 37 | 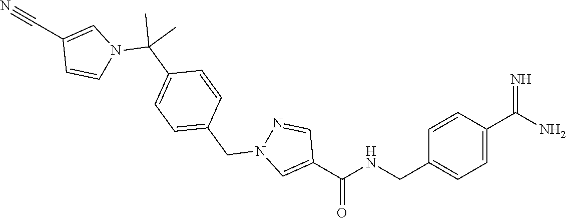 [SMILES: CC(C)(c1ccc(Cn2cc(C(=O)NCc3ccc(C(=N)N)cc3)cn2)cc1)n1ccc(C#N)c1] | | | |
| 38 | 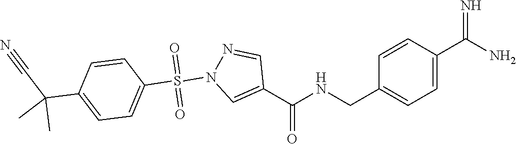 [SMILES: CC(C)(C#N)c1ccc(S(=O)(=O)n2cc(C(=O)NCc3ccc(C(=N)N)cc3)cn2)cc1] | | | |
| 39 | 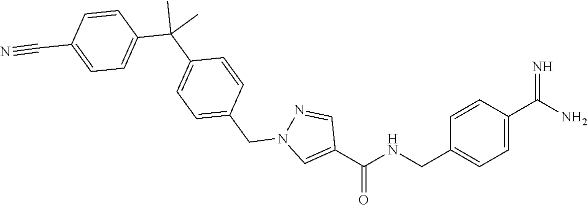 [SMILES: CC(C)(c1ccc(C#N)cc1)c1ccc(Cn2cc(C(=O)NCc3ccc(C(=N)N)cc3)cn2)cc1] | | | |
| 40 | 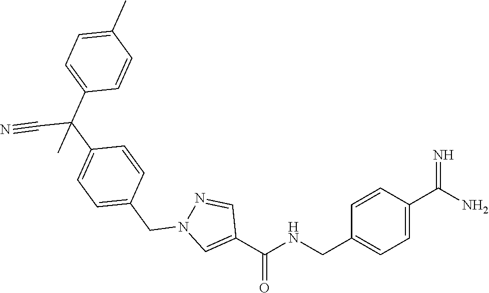 [SMILES: Cc1ccc(C(C)(C#N)c2ccc(Cn3cc(C(=O)NCc4ccc(C(=N)N)cc4)cn3)cc2)cc1] | | | |
| 41 | 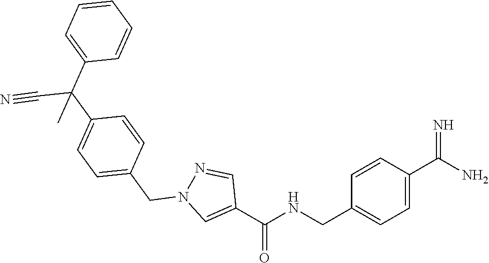 [SMILES: CC(C#N)(c1ccccc1)c1ccc(Cn2cc(C(=O)NCc3ccc(C(=N)N)cc3)cn2)cc1] | | | |
| 42 | 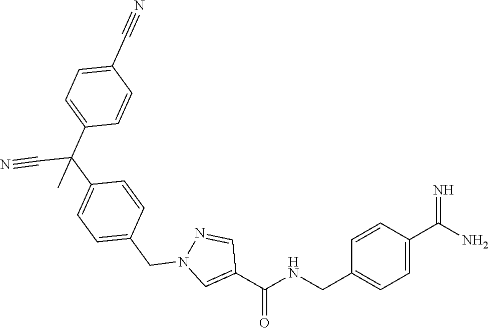 [SMILES: CC(C#N)(c1ccc(C#N)cc1)c1ccc(Cn2cc(C(=O)NCc3ccc(C(=N)N)cc3)cn2)cc1] | | | |
| 43 | 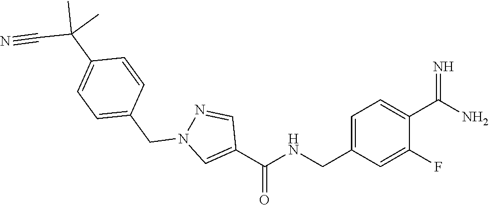 [SMILES: CC(C)(C#N)c1ccc(Cn2cc(C(=O)NCc3ccc(C(=N)N)c(F)c3)cn2)cc1] | B | A | A |
| 44 | 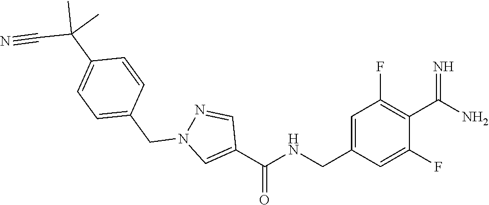 [SMILES: CC(C)(C#N)c1ccc(Cn2cc(C(=O)NCc3cc(F)c(C(=N)N)c(F)c3)cn2)cc1] | D | C | |
| 45 | 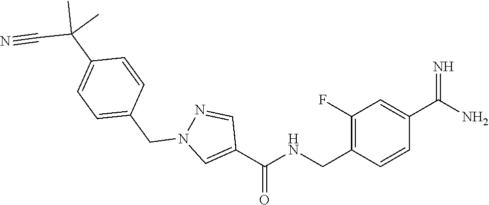 [SMILES: CC(C)(C#N)c1ccc(Cn2cc(C(=O)NCc3ccc(C(=N)N)cc3F)cn2)cc1] | A | A | B |
| 46 | 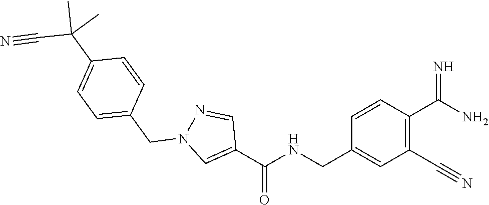 [SMILES: CC(C)(C#N)c1ccc(Cn2cc(C(=O)NCc3ccc(C(=N)N)c(C#N)c3)cn2)cc1] | | | |
| 47 | 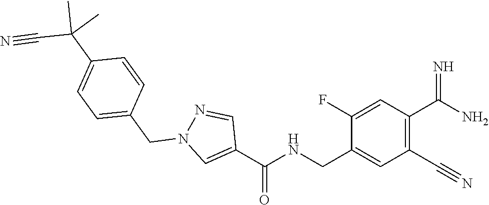 [SMILES: CC(C)(C#N)c1ccc(Cn2cc(C(=O)NCc3cc(C#N)c(C(=N)N)cc3F)cn2)cc1] | | | |
| 48 | 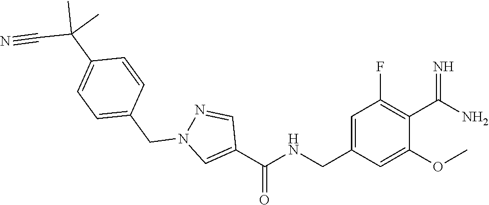 [SMILES: COc1cc(CNC(=O)c2cnn(Cc3ccc(C(C)(C)C#N)cc3)c2)cc(F)c1C(=N)N] | C | B | A |
| 49 | 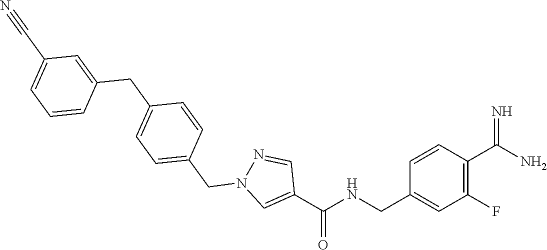 [SMILES: N#Cc1cccc(Cc2ccc(Cn3cc(C(=O)NCc4ccc(C(=N)N)c(F)c4)cn3)cc2)c1] | A | B | A |
| 50 | 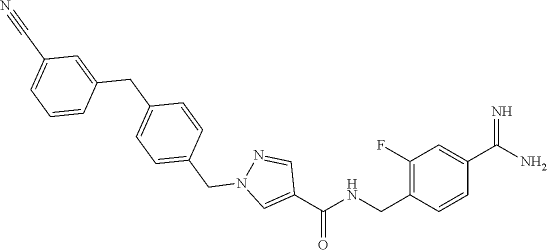 [SMILES: N#Cc1cccc(Cc2ccc(Cn3cc(C(=O)NCc4ccc(C(=N)N)cc4F)cn3)cc2)c1] | A | A | A |
| 52 | 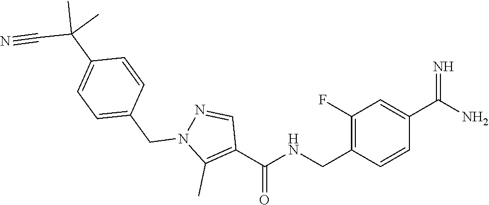 [SMILES: Cc1c(C(=O)NCc2ccc(C(=N)N)cc2F)cnn1Cc1ccc(C(C)(C)C#N)cc1] | | | |
| 53 | 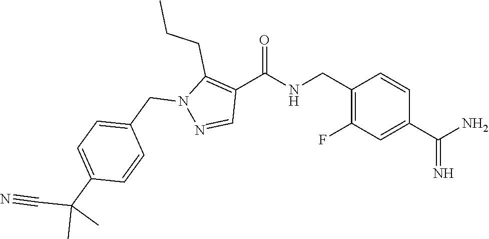 [SMILES: CCCc1c(C(=O)NCc2ccc(C(=N)N)cc2F)cnn1Cc1ccc(C(C)(C)C#N)cc1] | | | |
| 54 | 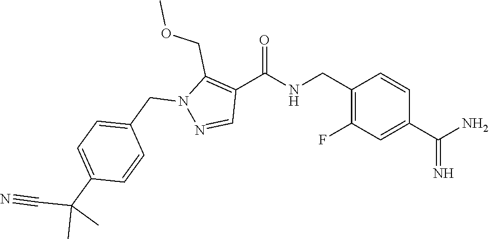 [SMILES: COCc1c(C(=O)NCc2ccc(C(=N)N)cc2F)cnn1Cc1ccc(C(C)(C)C#N)cc1] | | | |
| 55 | 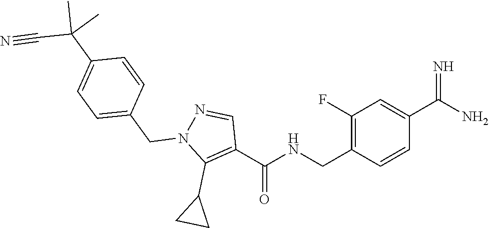 [SMILES: CC(C)(C#N)c1ccc(Cn2ncc(C(=O)NCc3ccc(C(=N)N)cc3F)c2C2CC2)cc1] | | | |
| 56 | 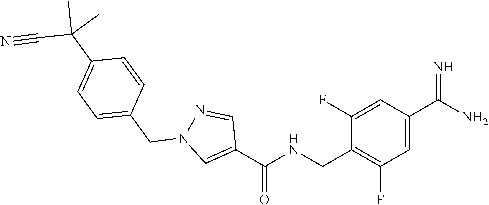 [SMILES: CC(C)(C#N)c1ccc(Cn2cc(C(=O)NCc3c(F)cc(C(=N)N)cc3F)cn2)cc1] | | | |
| 57 | 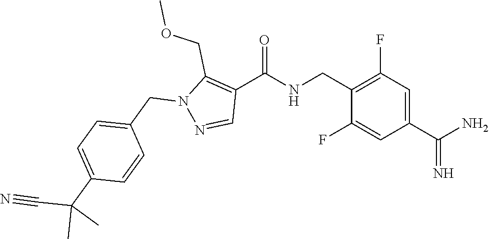 [SMILES: COCc1c(C(=O)NCc2c(F)cc(C(=N)N)cc2F)cnn1Cc1ccc(C(C)(C)C#N)cc1] | | | |
| 58 | 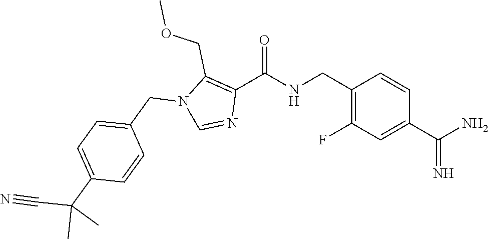 [SMILES: COCc1c(C(=O)NCc2ccc(C(=N)N)cc2F)ncn1Cc1ccc(C(C)(C)C#N)cc1] | | | |
| 59 | 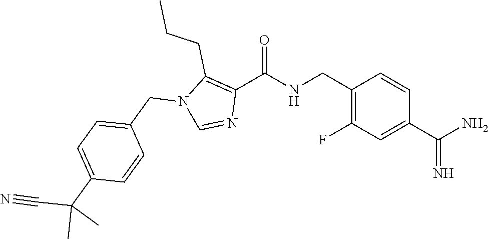 [SMILES: CCCc1c(C(=O)NCc2ccc(C(=N)N)cc2F)ncn1Cc1ccc(C(C)(C)C#N)cc1] | | | |
| 60 | 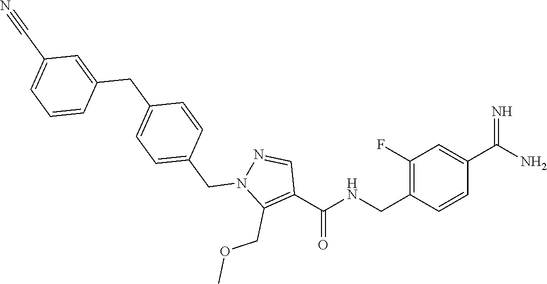 [SMILES: COCc1c(C(=O)NCc2ccc(C(=N)N)cc2F)cnn1Cc1ccc(Cc2cccc(C#N)c2)cc1] | | | |
| 61 | 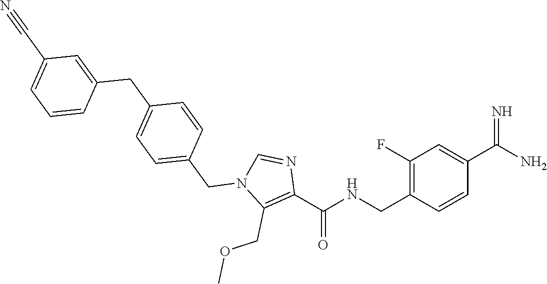 [SMILES: COCc1c(C(=O)NCc2ccc(C(=N)N)cc2F)ncn1Cc1ccc(Cc2cccc(C#N)c2)cc1] | | | |
| 62 | 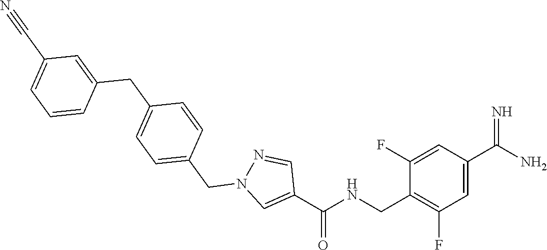 [SMILES: N#Cc1cccc(Cc2ccc(Cn3cc(C(=O)NCc4c(F)cc(C(=N)N)cc4F)cn3)cc2)c1] | | | |
| 63 | 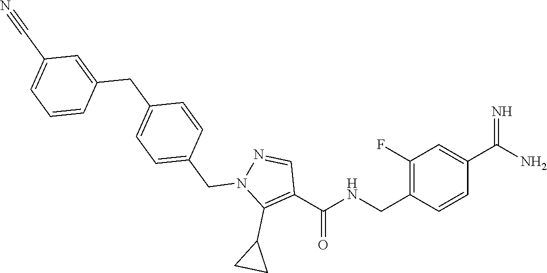 [SMILES: N#Cc1cccc(Cc2ccc(Cn3ncc(C(=O)NCc4ccc(C(=N)N)cc4F)c3C3CC3)cc2)c1] | | | |
| 64 | 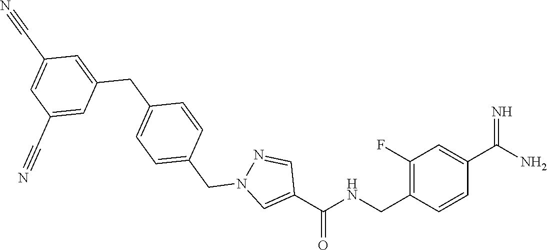 [SMILES: N#Cc1cc(C#N)cc(Cc2ccc(Cn3cc(C(=O)NCc4ccc(C(=N)N)cc4F)cn3)cc2)c1] | | | |
| 65 | 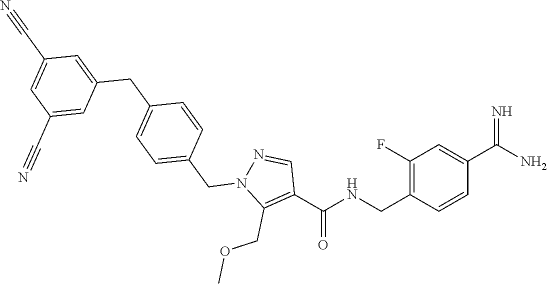 [SMILES: COCc1c(C(=O)NCc2ccc(C(=N)N)cc2F)cnn1Cc1ccc(Cc2cc(C#N)cc(C#N)c2)cc1] | | | |
| 66 | 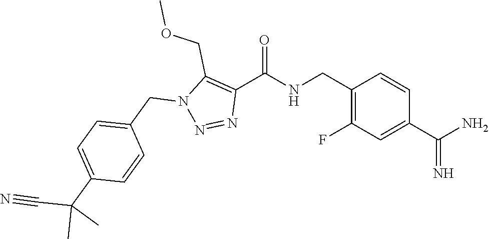 [SMILES: COCc1c(C(=O)NCc2ccc(C(=N)N)cc2F)nnn1Cc1ccc(C(C)(C)C#N)cc1] | | | |
| 67 | 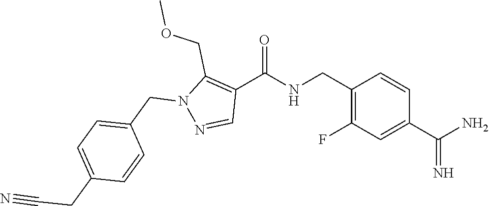 [SMILES: COCc1c(C(=O)NCc2ccc(C(=N)N)cc2F)cnn1Cc1ccc(CC#N)cc1] | | | |
| 68 | 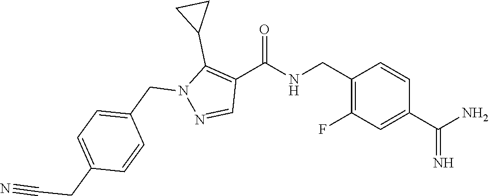 [SMILES: N#CCc1ccc(Cn2ncc(C(=O)NCc3ccc(C(=N)N)cc3F)c2C2CC2)cc1] | | | |
| 69 | 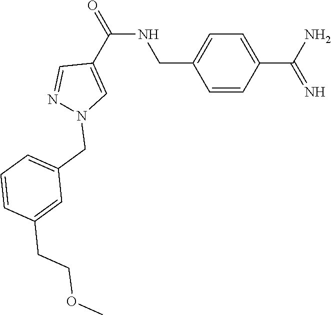 [SMILES: COCCc1cccc(Cn2cc(C(=O)NCc3ccc(C(=N)N)cc3)cn2)c1] | B | A | |
| 70 | 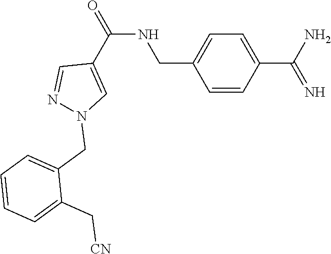 [SMILES: N#CCc1ccccc1Cn1cc(C(=O)NCc2ccc(C(=N)N)cc2)cn1] | C | B | |
| 71 | 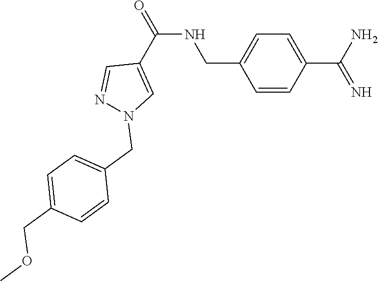 [SMILES: COCc1ccc(Cn2cc(C(=O)NCc3ccc(C(=N)N)cc3)cn2)cc1] | A | A | |
| 72 | 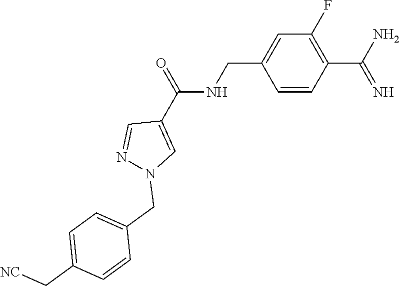 [SMILES: N#CCc1ccc(Cn2cc(C(=O)NCc3ccc(C(=N)N)c(F)c3)cn2)cc1] | C | B | |
| 73 | 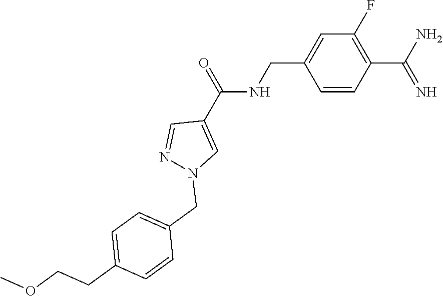 [SMILES: COCCc1ccc(Cn2cc(C(=O)NCc3ccc(C(=N)N)c(F)c3)cn2)cc1] | B | A | |
| 74 | 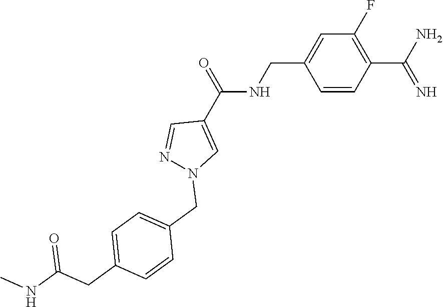 [SMILES: CNC(=O)Cc1ccc(Cn2cc(C(=O)NCc3ccc(C(=N)N)c(F)c3)cn2)cc1] | B | B | |
| 75 | 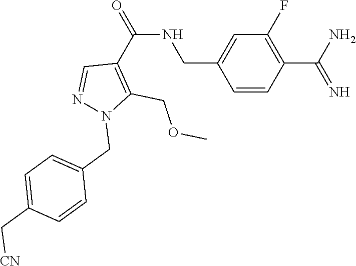 [SMILES: COCc1c(C(=O)NCc2ccc(C(=N)N)c(F)c2)cnn1Cc1ccc(CC#N)cc1] | D | C | |
| 78 | 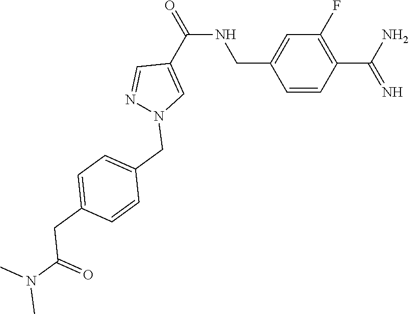 [SMILES: CN(C)C(=O)Cc1ccc(Cn2cc(C(=O)NCc3ccc(C(=N)N)c(F)c3)cn2)cc1] | | | |
| 81 | 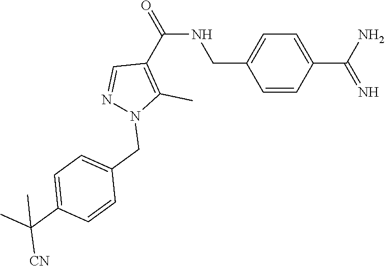 [SMILES: Cc1c(C(=O)NCc2ccc(C(=N)N)cc2)cnn1Cc1ccc(C(C)(C)C#N)cc1] | A | A | |
| 82 | 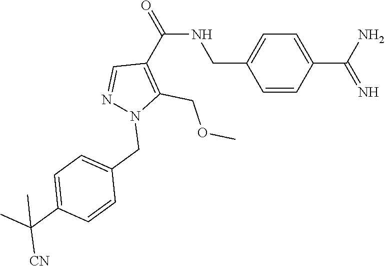 [SMILES: COCc1c(C(=O)NCc2ccc(C(=N)N)cc2)cnn1Cc1ccc(C(C)(C)C#N)cc1] | C | A | |
| 83 | 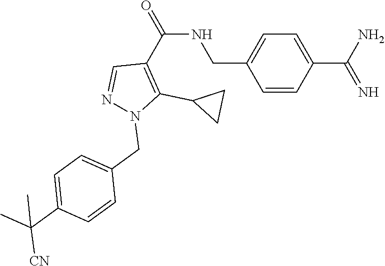 [SMILES: CC(C)(C#N)c1ccc(Cn2ncc(C(=O)NCc3ccc(C(=N)N)cc3)c2C2CC2)cc1] | C | B | |
| 84 | 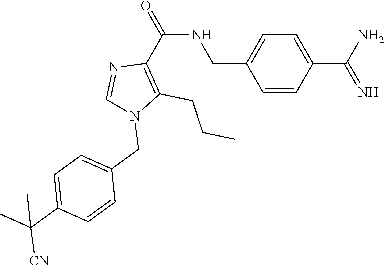 [SMILES: CCCc1c(C(=O)NCc2ccc(C(=N)N)cc2)ncn1Cc1ccc(C(C)(C)C#N)cc1] | D | D | |
| 85 | 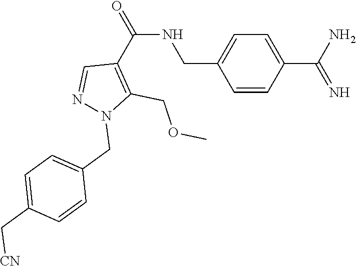 [SMILES: COCc1c(C(=O)NCc2ccc(C(=N)N)cc2)cnn1Cc1ccc(CC#N)cc1] | C | B | |
| 86 | 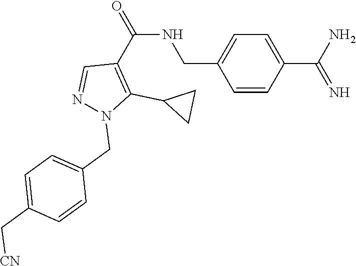 [SMILES: N#CCc1ccc(Cn2ncc(C(=O)NCc3ccc(C(=N)N)cc3)c2C2CC2)cc1] | C | B | |
| 87 | 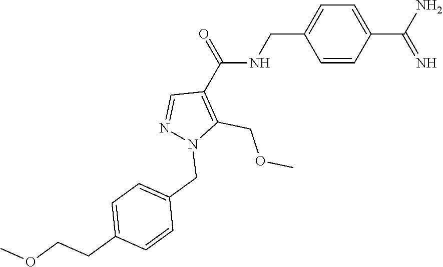 [SMILES: COCCc1ccc(Cn2ncc(C(=O)NCc3ccc(C(=N)N)cc3)c2COC)cc1] | C | A | A |

It is understood that the examples and embodiments described herein are for illustrative purposes only and that various modifications or changes in light thereof will be suggested to persons skilled in the art and are to be included within the spirit and purview of this application and scope of the appended claims. All publications, patents, and patent applications cited herein are hereby incorporated by reference in their entirety for all purposes.

1. A compound of formula (IA) or a pharmaceutically acceptable salt thereof: 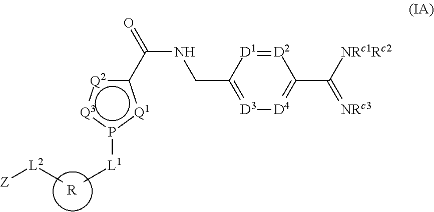
wherein
D¹ is N or CR¹, D² is N or CR², D³ is N or CR³, and D⁴ is N or CR⁴, wherein no more than three of D¹, D², D³, and D⁴ are simultaneously N;
R¹, R², R³, and R⁴ are independently selected from the group consisting of H, C₂-C₆-alkenyl, C₁-C₆-haloalkyl, halo, NR^{a}R^{b}, OR^{a}, —R^{a} C(O)R^{b}, —C(O)R^{a}, —C(O)halo, —OC(O)R^{a}, —OC(O)OR^{a}, —C(O)OR^{a}, C₆-C₁₀-aryl, CN, —S(O)₀₋₂R^{a}, —S(O)₂OR^{a}, and NO₂;
each R^{a} and R^{b} is independently selected from the group consisting of H, C₁-C₆-alkyl, and C₁-C₆-haloalkyl;
R^{c1}, R^{c2}, and R^{c3} are independently selected from group consisting of H, C₁-C₆-alkyl, and C₁-C₆-haloalkyl;
Q₁, Q₂, and Q₃ are independently selected from the group consisting of CR⁵, N, O and S;
R⁵ is selected from the group consisting of H, C₁-C₆-alkyl, OR^{a}, —(C₁-C₆-alkyl) OR^{a}, and C₃-C₁₀-cycloalkyl;
P is C or N;
L¹ is —SO₂— or —C₁-C₈-alkylene-; 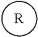
is a bivalent monocyclic or bicyclic moiety selected from the group consisting of C₃-C₁₀-cycloalkyl, C₆-C₁₀-aryl, 3- to 10-membered heterocycloalkyl (wherein 1-4 ring members are independently selected from N, O, and S), 5- to 10-membered heteroaryl (wherein 1-4 heteroaryl members are independently selected from N, O, and S), and fused combinations thereof,
L₂ is selected from the group consisting of —C₁-C₆-alkylene, —C₂-C₈-alkenylene, —C₂-C₈-alkynylene, and a bivalent moiety selected from the group consisting of C₃-C₁₀-cycloalkyl, C₃-C₁₀-cycloalkenyl, C₆-C₁₀-aryl, 5- to 10-membered heteroaryl (wherein 1-4 heteroaryl members are independently selected from N, O, and S), (C₁-C₈-alkyl) C₃-C₁₀-cycloalkyl, (C₁-C₆-alkyl) C₃-C₁₀-cycloalkenyl, (C₁-C₆-alkyl) C₆-C₁₀-aryl, and (C₁-C₈-alkyl) 5- to 10-membered heteroaryl (wherein 1-4 heteroaryl members are independently selected from N, O, and S), wherein cycloalkyl, cycloalkenyl, aryl, and heteroaryl are monocyclic or bicyclic;
wherein L₂ is optionally substituted by 1 to 3 substituents selected from the group consisting of C₁-C₆-alkyl, C₁-C₆-haloalkyl, and CN;
Z is selected from the group consisting of —OR^{c}, —OC(O)R^{c}, —OC(O)NR^{c}R^{d}, —S(O)₀₋₂R^{c}, —CN, —C(O)R^{c}, —CN, —C(O)OR^{c}, —C(O)NR^{c}R^{d}, —C(S)R^{c}, —R^{c}R^{d}, =NR^{c}, —R^{c}C(O)NR^{c}R^{d}, —R^{c}CO₂R^{d}, —NR^{c}—NO, —NO₂, —R^{c}—OR_{d}, —N═C═O, —N═C═S, and —NR^{c}—NR^{c}R^{d}; and
each instance of R^{c} and R^{d} is independently selected from H, C₁-C₆-alkyl, C₁-C₆-haloalkyl, and C₆-C₁₀-aryl.

2. The compound or pharmaceutically acceptable salt thereof according to claim 1,
wherein no more than one of D¹, D², D³, and D⁴ is N.

3. The compound or pharmaceutically acceptable salt thereof according to claim 1, wherein the ring containing D¹, D², D³, and D⁴ is selected from the group consisting of:

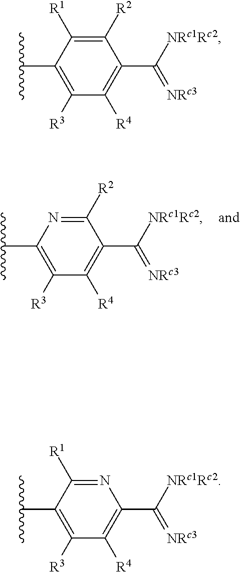

4. The compound or pharmaceutically acceptable salt thereof according to claim 3, wherein the ring containing D¹, D², D³, and D⁴ is

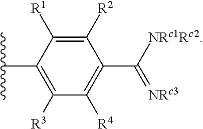

5. The compound or pharmaceutically acceptable salt thereof according to claim 4, wherein R¹, R², R³, and R⁴ are independently selected from the group consisting of H, OR^{a}, halo, and CN.

6. The compound or pharmaceutically acceptable salt thereof according to claim 5, wherein R¹, R², R³, and R⁴ are independently selected from the group consisting of H and halo.

7. The compound or pharmaceutically acceptable salt thereof according to claim 6, wherein at least one of R¹, R², R³, and R⁴ is halo.

8. The compound or pharmaceutically acceptable salt thereof according to claim 6, wherein each of R¹, R², R³, and R⁴ is H.

9. The compound or pharmaceutically acceptable salt thereof according to claim 1, wherein each of R^{c1}, R^{c2}, and R^{c3} is H.

10. The compound or pharmaceutically acceptable salt thereof according to claim 1, wherein
Q₁ is C;
each of Q₂ and Q₃ is independently selected from the group consisting of CR⁵, N, O and S;
and P is selected from the group consisting of C and N.

11. The compound or pharmaceutically acceptable salt thereof according to claim 10, wherein P is N.

12. The compound or pharmaceutically acceptable salt thereof according to claim 11, wherein Q² is CR⁵ and Q₃ is N.

13. The compound or pharmaceutically acceptable salt thereof according to claim 11, wherein Q² is N and Q₃ is CR⁵.

14. The compound or pharmaceutically acceptable salt thereof according to claim 1, wherein L¹ is —C₁-C₈-alkylene-.

15. The compound or pharmaceutically acceptable salt thereof according to claim 14, wherein L¹ is —C₁-C₃-alkylene-.

16. The compound or pharmaceutically acceptable salt thereof according to claim 1, wherein 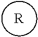
is bivalent monocyclic C₆-C₁₀-aryl.

17. The compound or pharmaceutically acceptable salt thereof according to claim 16, wherein 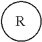
is

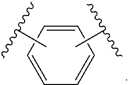

18. The compound or pharmaceutically acceptable salt thereof according to claim 17, wherein L₂ is selected from —C₁-C₈-alkylene, C₆-C₁₀-aryl and (C₁-C₆-alkyl) C₆-C₁₀-aryl.

19. The compound or pharmaceutically acceptable salt thereof according to claim 18, wherein L₂ is —C₁-C₈-alkylene.

20. The compound or pharmaceutically acceptable salt thereof according to claim 19 wherein L₂ is C₁-C₃-alkylene.

21. The compound or pharmaceutically acceptable salt thereof according to claim 1, wherein L₂ is C₆-C₁₀-aryl.

22. The compound or pharmaceutically acceptable salt thereof according to claim 21, wherein L₂ is phenyl.

23. The compound or pharmaceutically acceptable salt thereof according to claim 1, wherein Z is selected from the group consisting of —OR^{c}, CN, —C(O)OR^{c}, and —C(O)NR^{c}R⁴.

24. The compound or pharmaceutically acceptable salt thereof according to claim 23, wherein Z is CN.

25. The compound or pharmaceutically acceptable salt thereof according to claim 23, wherein each R^{c} and R^{d} is independently H or C₁-C₆-alkyl.

26. The compound or pharmaceutically acceptable salt thereof according to claim 23, wherein Z is selected from the group consisting of OH, OCH₃, —COOH, —C(O)NH₂, and —C(O)NHCH₃.

27. The compound or pharmaceutically acceptable salt thereof according to claim 1, wherein the compound is of formula (I): 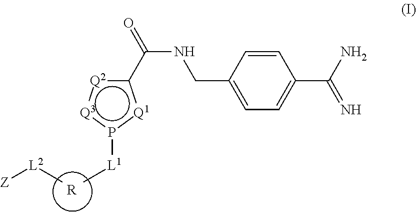
wherein
Q₁, Q₂, and Q₃ are selected from the group consisting of C, N, O, and S;
P is selected from the group consisting of C and N;
L¹ is a linking group selected from the group consisting of a C₁-C₈ alkyl linker and SO₂; 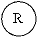
is
i) a cyclic hydrocarbon, bicyclic hydrocarbon, or heterocycle containing up to 10 atoms consisting of C or N; or
ii) selected from the group consisting of cyclopentyl, cyclohexyl, phenyl, naphthyl, pyridine, pyrimidine, indene, 2,3-dihydro-1H-indene or any saturated and unsaturated cyclic hydrocarbon or heterocycle thereof;
L₂ is
i) a hydrocarbon that does not contain a double bond to O or S,
ii) a hydrocarbon and does not contain a heteroatom such as O, N, or S; or
iii) a linking group selected from the group consisting of a C₁-C₈ alkyl linker comprising a saturated hydrocarbon, an unsaturated hydrocarbon, a branched hydrocarbon, a cyclic hydrocarbon and combinations thereof; a cyclic hydrocarbon selected from the group consisting of cyclopentyl, cyclohexyl, phenyl, naphthyl, indene, and 2,3-dihydro-1H-indene or any saturated or unsaturated cyclic hydrocarbon thereof;
Z is
i) selected from the group consisting of —OH, —OR′, —OC(O) H, —OC(O)R′, —OC(O)NH₂, —OC(O)NHR′, —OC(O)NH(R′)₂, —SH, —SR′, —S(O)R′, —S(O)₂R′, —CN, —C(O) H, —C(O)R′, —C(O)OH, —C(O)OR′, —C(O)NH₂, —C(O)NHR′, —C(O)NH(R′)₂, —C(S)R′, —NH₂, —NH₂R′, —R′2, —NH, —R′, —NHC(O)NH₂, —R′C(O)NH₂, —NR′C(O)NHR′, —R′C(O)N(R′)₂, —NHCO₂H, —NHCO₂R′, —R′CO₂R′, —NH—NO, —NR′—NO, —NO₂, —NH—OH, —OH, —R′—OR′, —N═C═O, —N═C═S, —NH—NH₂, and —NH—NHR′, wherein each R′ is independently alkyl or alkyl halide, or
ii) is not a halogen or hydrogen.

28. The compound or pharmaceutically acceptable salt thereof according to claim 1, wherein the compound is selected from the following table:

| | |
|---|---|
| 1 | 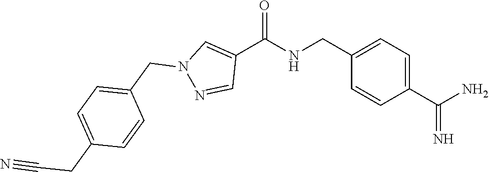 [SMILES: N#CCc1ccc(Cn2cc(C(=O)NCc3ccc(C(=N)N)cc3)cn2)cc1] |
| 2 | 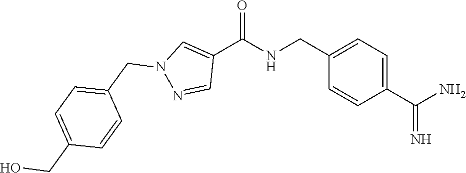 [SMILES: N=C(N)c1ccc(CNC(=O)c2cnn(Cc3ccc(CO)cc3)c2)cc1] |
| 3 | 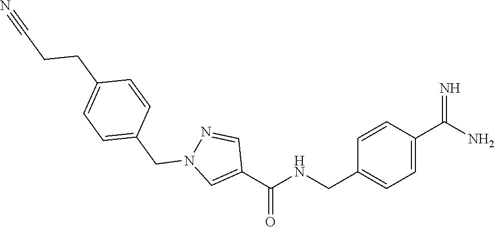 [SMILES: N#CCCc1ccc(Cn2cc(C(=O)NCc3ccc(C(=N)N)cc3)cn2)cc1] |
| 4 | 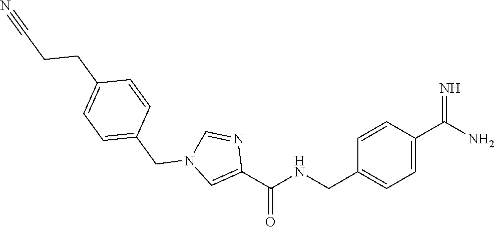 [SMILES: N#CCCc1ccc(Cn2cnc(C(=O)NCc3ccc(C(=N)N)cc3)c2)cc1] |
| 5 | 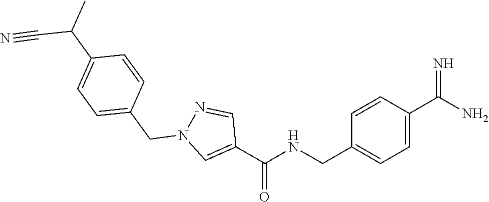 [SMILES: CC(C#N)c1ccc(Cn2cc(C(=O)NCc3ccc(C(=N)N)cc3)cn2)cc1] |
| 6 | 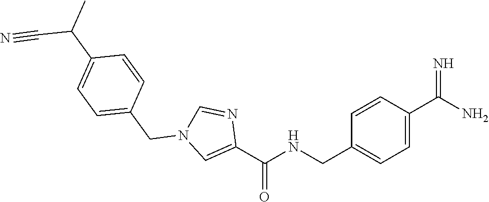 [SMILES: CC(C#N)c1ccc(Cn2cnc(C(=O)NCc3ccc(C(=N)N)cc3)c2)cc1] |
| 7 | 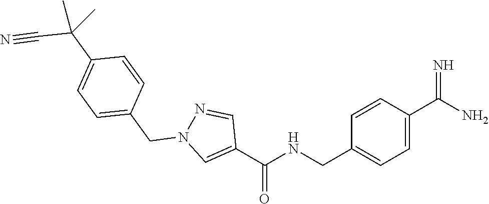 [SMILES: CC(C)(C#N)c1ccc(Cn2cc(C(=O)NCc3ccc(C(=N)N)cc3)cn2)cc1] |
| 8 | 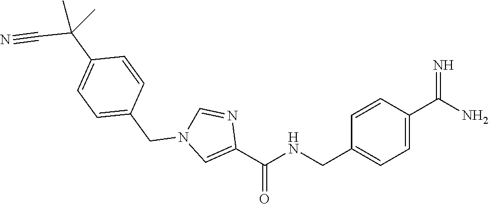 [SMILES: CC(C)(C#N)c1ccc(Cn2cnc(C(=O)NCc3ccc(C(=N)N)cc3)c2)cc1] |
| 9 | 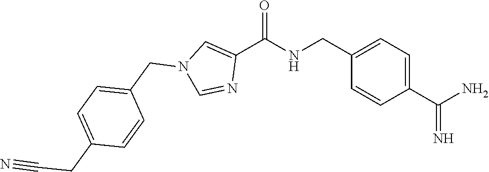 [SMILES: N#CCc1ccc(Cn2cnc(C(=O)NCc3ccc(C(=N)N)cc3)c2)cc1] |
| 10 | 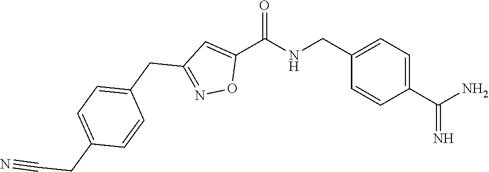 [SMILES: N#CCc1ccc(Cc2cc(C(=O)NCc3ccc(C(=N)N)cc3)on2)cc1] |
| 11 | 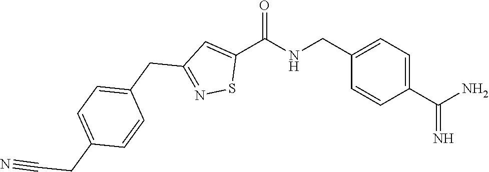 [SMILES: N#CCc1ccc(Cc2cc(C(=O)NCc3ccc(C(=N)N)cc3)sn2)cc1] |
| 12 | 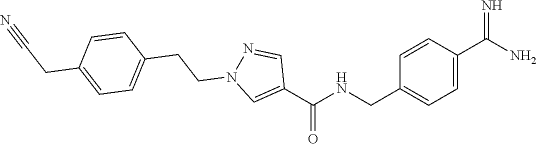 [SMILES: N#CCc1ccc(CCn2cc(C(=O)NCc3ccc(C(=N)N)cc3)cn2)cc1] |
| 13 | 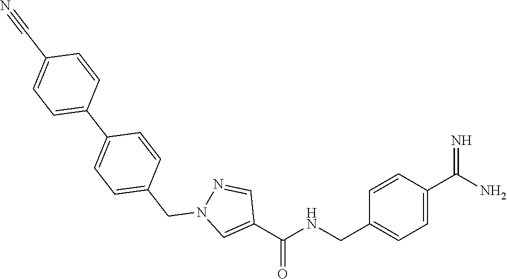 [SMILES: N#Cc1ccc(-c2ccc(Cn3cc(C(=O)NCc4ccc(C(=N)N)cc4)cn3)cc2)cc1] |
| 14 | 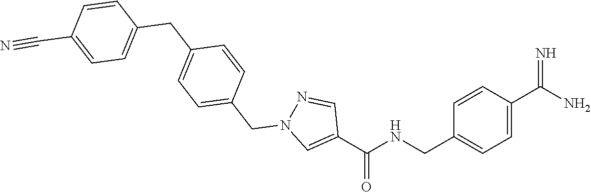 [SMILES: N#Cc1ccc(Cc2ccc(Cn3cc(C(=O)NCc4ccc(C(=N)N)cc4)cn3)cc2)cc1] |
| 15 | 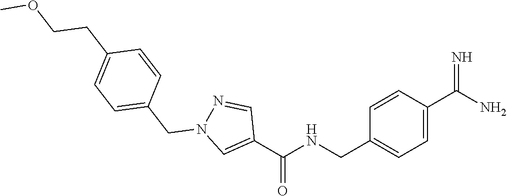 [SMILES: COCCc1ccc(Cn2cc(C(=O)NCc3ccc(C(=N)N)cc3)cn2)cc1] |
| 16 | 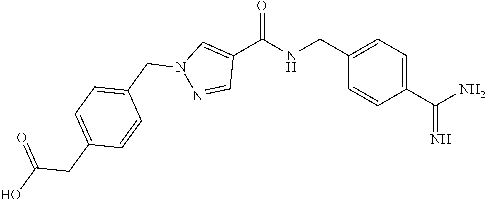 [SMILES: N=C(N)c1ccc(CNC(=O)c2cnn(Cc3ccc(CC(=O)O)cc3)c2)cc1] |
| 17 | 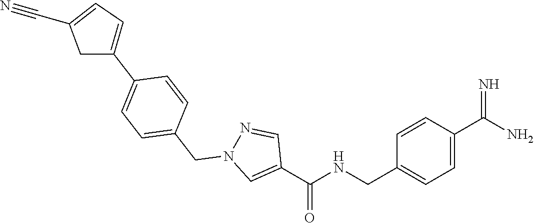 [SMILES: N#CC1=CC=C(c2ccc(Cn3cc(C(=O)NCc4ccc(C(=N)N)cc4)cn3)cc2)C1] |
| 18 | 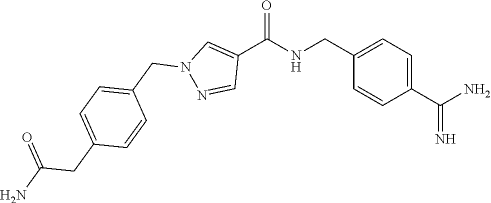 [SMILES: N=C(N)c1ccc(CNC(=O)c2cnn(Cc3ccc(CC(N)=O)cc3)c2)cc1] |
| 19 | 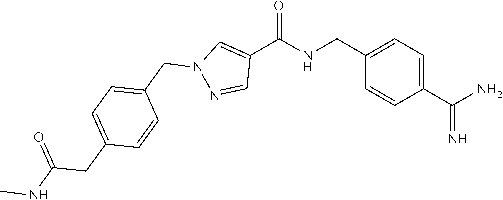 [SMILES: CNC(=O)Cc1ccc(Cn2cc(C(=O)NCc3ccc(C(=N)N)cc3)cn2)cc1] |
| 20 | 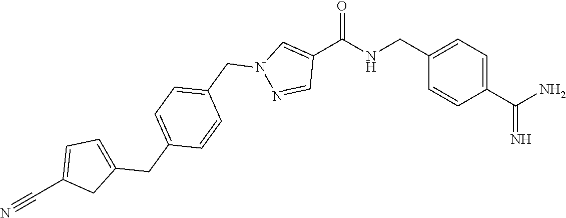 [SMILES: N#CC1=CC=C(Cc2ccc(Cn3cc(C(=O)NCc4ccc(C(=N)N)cc4)cn3)cc2)C1] |
| 21 | 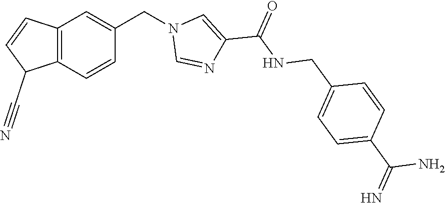 [SMILES: N#CC1C=Cc2cc(Cn3cnc(C(=O)NCc4ccc(C(=N)N)cc4)c3)ccc21] |
| 22 | 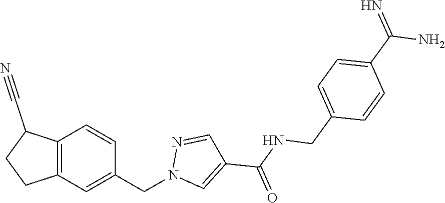 [SMILES: N#CC1CCc2cc(Cn3cc(C(=O)NCc4ccc(C(=N)N)cc4)cn3)ccc21] |
| 23 | 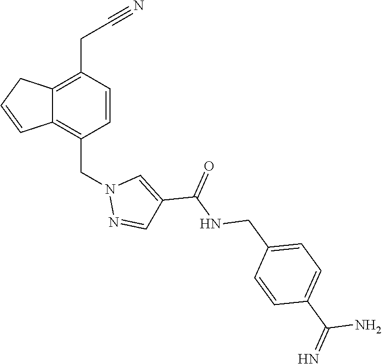 [SMILES: N#CCc1ccc(Cn2cc(C(=O)NCc3ccc(C(=N)N)cc3)cn2)c2c1CC=C2] |
| 24 | 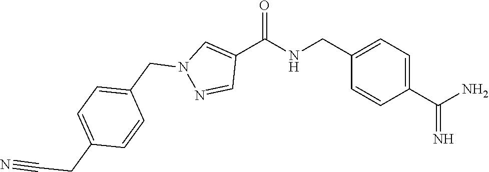 [SMILES: N#CCc1ccc(Cn2cc(C(=O)NCc3ccc(C(=N)N)cc3)cn2)cc1] |
| 25 | 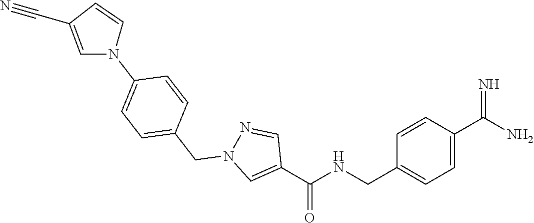 [SMILES: N#Cc1ccn(-c2ccc(Cn3cc(C(=O)NCc4ccc(C(=N)N)cc4)cn3)cc2)c1] |
| 26 | 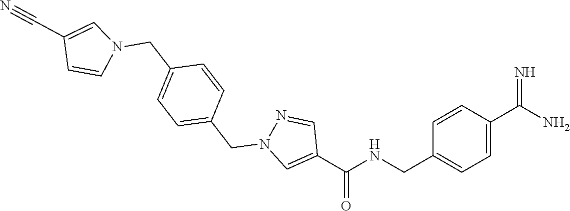 [SMILES: N#Cc1ccn(Cc2ccc(Cn3cc(C(=O)NCc4ccc(C(=N)N)cc4)cn3)cc2)c1] |
| 27 | 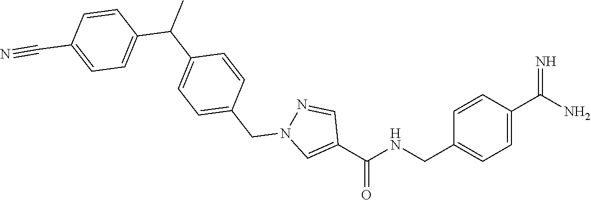 [SMILES: CC(c1ccc(C#N)cc1)c1ccc(Cn2cc(C(=O)NCc3ccc(C(=N)N)cc3)cn2)cc1] |
| 28 | 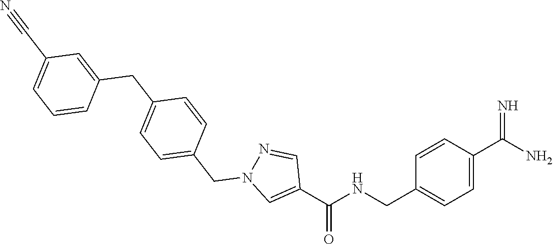 [SMILES: N#Cc1cccc(Cc2ccc(Cn3cc(C(=O)NCc4ccc(C(=N)N)cc4)cn3)cc2)c1] |
| 29 | 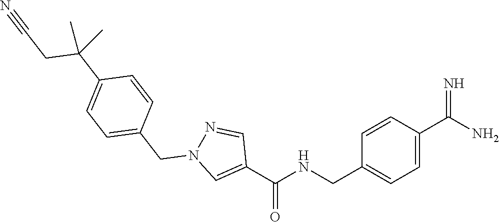 [SMILES: CC(C)(CC#N)c1ccc(Cn2cc(C(=O)NCc3ccc(C(=N)N)cc3)cn2)cc1] |
| 30 | 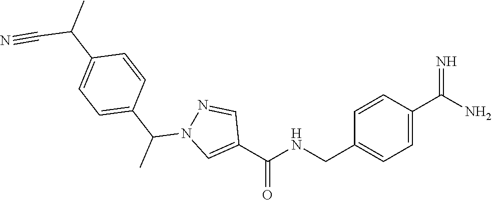 [SMILES: CC(C#N)c1ccc(C(C)n2cc(C(=O)NCc3ccc(C(=N)N)cc3)cn2)cc1] |
| 31 | 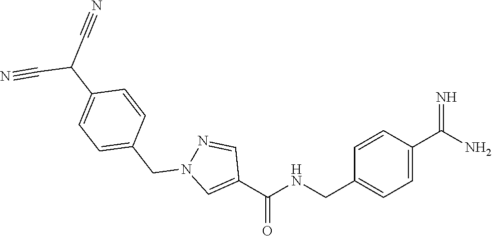 [SMILES: N#CC(C#N)c1ccc(Cn2cc(C(=O)NCc3ccc(C(=N)N)cc3)cn2)cc1] |
| 32 | 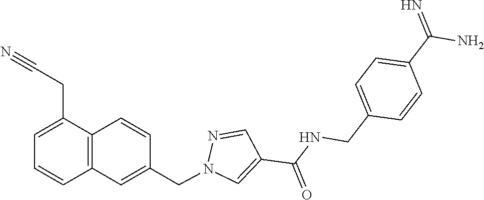 [SMILES: N#CCc1cccc2cc(Cn3cc(C(=O)NCc4ccc(C(=N)N)cc4)cn3)ccc12] |
| 33 | 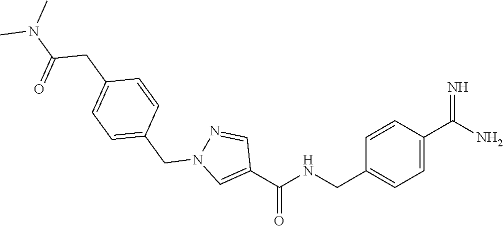 [SMILES: CN(C)C(=O)Cc1ccc(Cn2cc(C(=O)NCc3ccc(C(=N)N)cc3)cn2)cc1] |
| 34 | 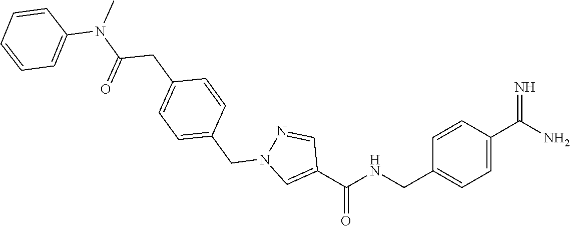 [SMILES: CN(C(=O)Cc1ccc(Cn2cc(C(=O)NCc3ccc(C(=N)N)cc3)cn2)cc1)c1ccccc1] |
| 35 | 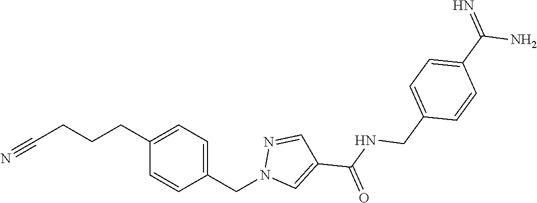 [SMILES: N#CCCCc1ccc(Cn2cc(C(=O)NCc3ccc(C(=N)N)cc3)cn2)cc1] |
| 36 | 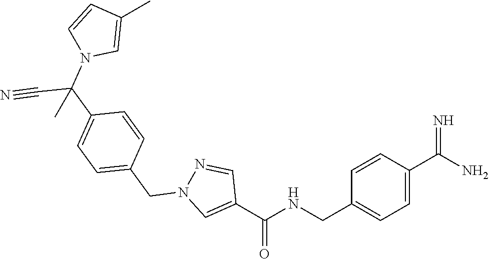 [SMILES: Cc1ccn(C(C)(C#N)c2ccc(Cn3cc(C(=O)NCc4ccc(C(=N)N)cc4)cn3)cc2)c1] |
| 37 | 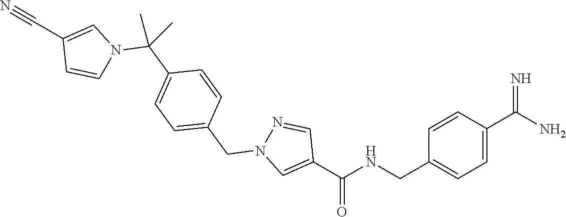 [SMILES: CC(C)(c1ccc(Cn2cc(C(=O)NCc3ccc(C(=N)N)cc3)cn2)cc1)n1ccc(C#N)c1] |
| 38 | 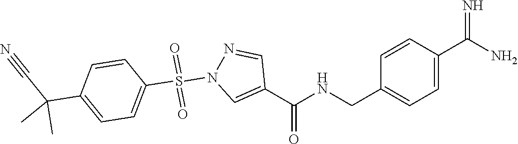 [SMILES: CC(C)(C#N)c1ccc(S(=O)(=O)n2cc(C(=O)NCc3ccc(C(=N)N)cc3)cn2)cc1] |
| 39 | 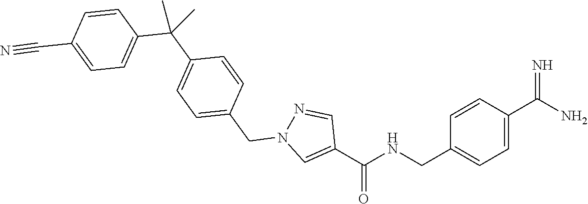 [SMILES: CC(C)(c1ccc(C#N)cc1)c1ccc(Cn2cc(C(=O)NCc3ccc(C(=N)N)cc3)cn2)cc1] |
| 40 | 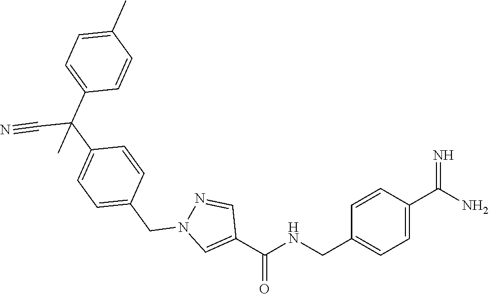 [SMILES: Cc1ccc(C(C)(C#N)c2ccc(Cn3cc(C(=O)NCc4ccc(C(=N)N)cc4)cn3)cc2)cc1] |
| 41 | 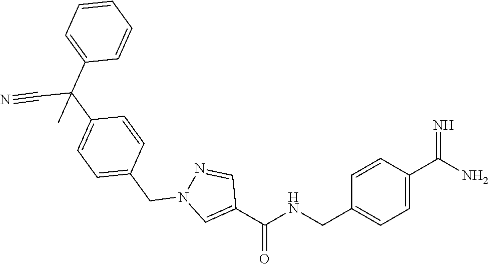 [SMILES: CC(C#N)(c1ccccc1)c1ccc(Cn2cc(C(=O)NCc3ccc(C(=N)N)cc3)cn2)cc1] |
| 42 | 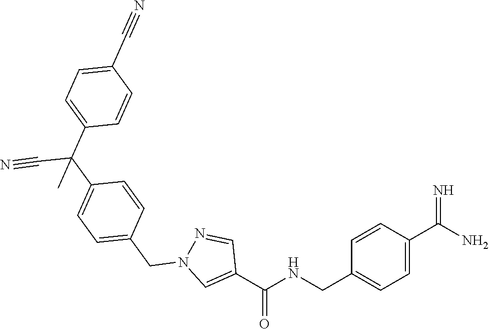 [SMILES: CC(C#N)(c1ccc(C#N)cc1)c1ccc(Cn2cc(C(=O)NCc3ccc(C(=N)N)cc3)cn2)cc1] |
| 43 | 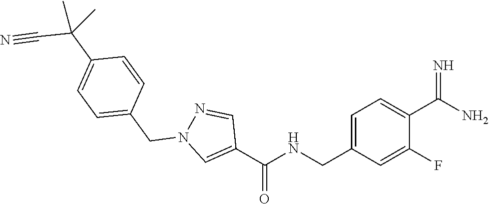 [SMILES: CC(C)(C#N)c1ccc(Cn2cc(C(=O)NCc3ccc(C(=N)N)c(F)c3)cn2)cc1] |
| 44 | 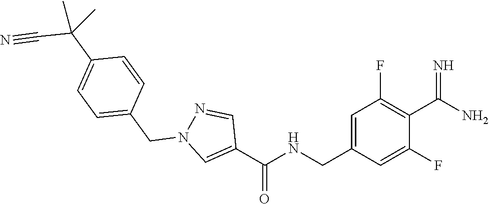 [SMILES: CC(C)(C#N)c1ccc(Cn2cc(C(=O)NCc3cc(F)c(C(=N)N)c(F)c3)cn2)cc1] |
| 45 | 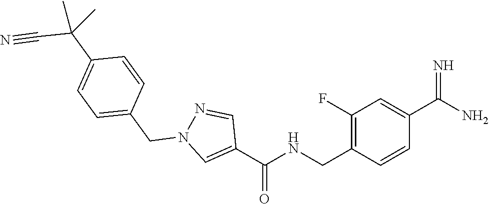 [SMILES: CC(C)(C#N)c1ccc(Cn2cc(C(=O)NCc3ccc(C(=N)N)cc3F)cn2)cc1] |
| 46 | 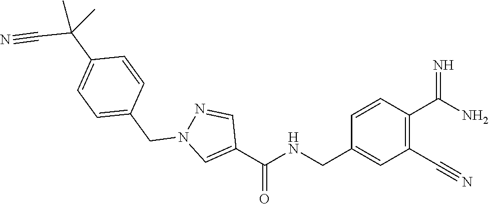 [SMILES: CC(C)(C#N)c1ccc(Cn2cc(C(=O)NCc3ccc(C(=N)N)c(C#N)c3)cn2)cc1] |
| 47 | 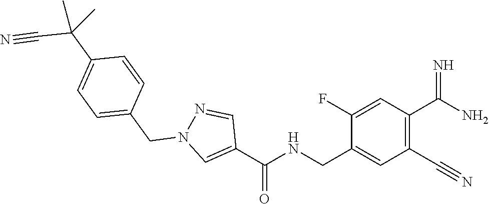 [SMILES: CC(C)(C#N)c1ccc(Cn2cc(C(=O)NCc3cc(C#N)c(C(=N)N)cc3F)cn2)cc1] |
| 48 | 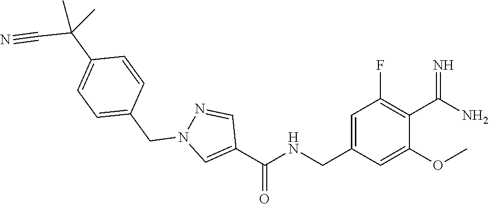 [SMILES: COc1cc(CNC(=O)c2cnn(Cc3ccc(C(C)(C)C#N)cc3)c2)cc(F)c1C(=N)N] |
| 49 | 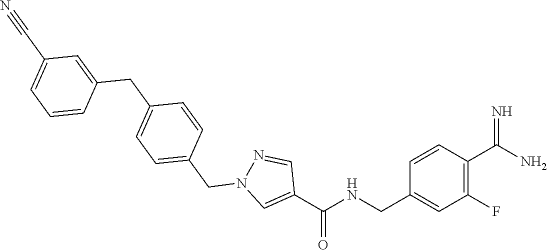 [SMILES: N#Cc1cccc(Cc2ccc(Cn3cc(C(=O)NCc4ccc(C(=N)N)c(F)c4)cn3)cc2)c1] |
| 50 | 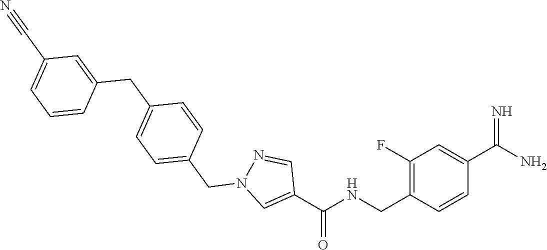 [SMILES: N#Cc1cccc(Cc2ccc(Cn3cc(C(=O)NCc4ccc(C(=N)N)cc4F)cn3)cc2)c1] |
| 52 | 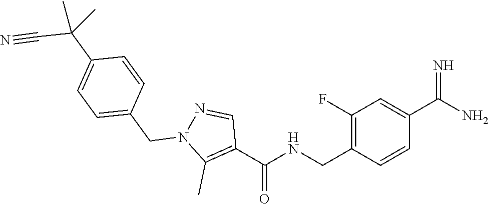 [SMILES: Cc1c(C(=O)NCc2ccc(C(=N)N)cc2F)cnn1Cc1ccc(C(C)(C)C#N)cc1] |
| 53 | 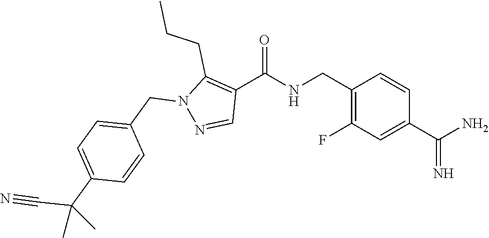 [SMILES: CCCc1c(C(=O)NCc2ccc(C(=N)N)cc2F)cnn1Cc1ccc(C(C)(C)C#N)cc1] |
| 54 | 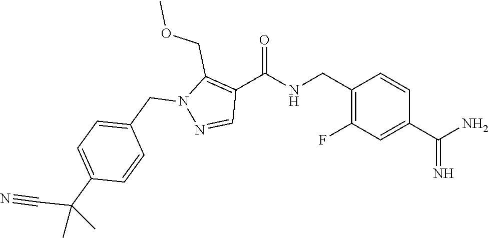 [SMILES: COCc1c(C(=O)NCc2ccc(C(=N)N)cc2F)cnn1Cc1ccc(C(C)(C)C#N)cc1] |
| 55 | 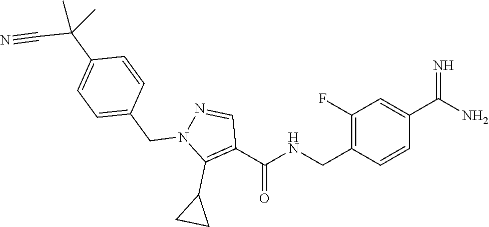 [SMILES: CC(C)(C#N)c1ccc(Cn2ncc(C(=O)NCc3ccc(C(=N)N)cc3F)c2C2CC2)cc1] |
| 56 | 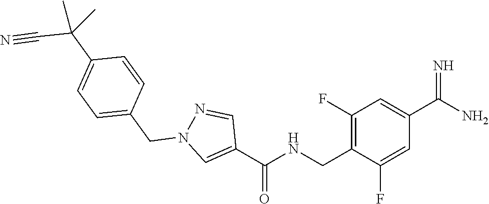 [SMILES: CC(C)(C#N)c1ccc(Cn2cc(C(=O)NCc3c(F)cc(C(=N)N)cc3F)cn2)cc1] |
| 57 | 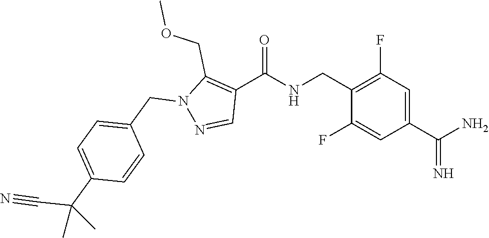 [SMILES: COCc1c(C(=O)NCc2c(F)cc(C(=N)N)cc2F)cnn1Cc1ccc(C(C)(C)C#N)cc1] |
| 58 | 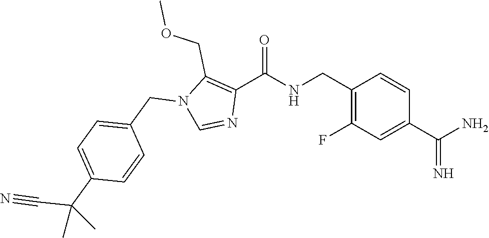 [SMILES: COCc1c(C(=O)NCc2ccc(C(=N)N)cc2F)ncn1Cc1ccc(C(C)(C)C#N)cc1] |
| 59 | 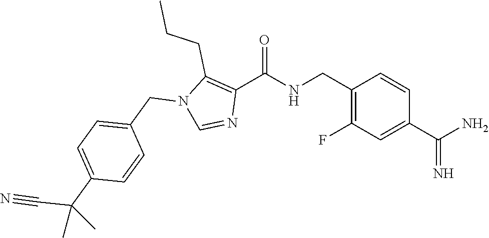 [SMILES: CCCc1c(C(=O)NCc2ccc(C(=N)N)cc2F)ncn1Cc1ccc(C(C)(C)C#N)cc1] |
| 60 | 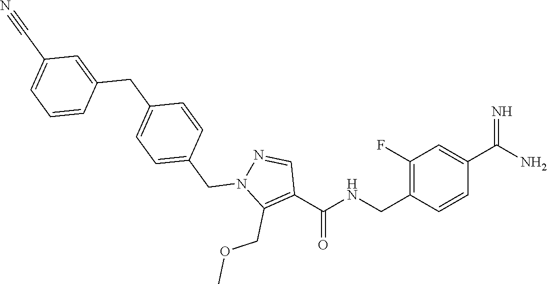 [SMILES: COCc1c(C(=O)NCc2ccc(C(=N)N)cc2F)cnn1Cc1ccc(Cc2cccc(C#N)c2)cc1] |
| 61 | 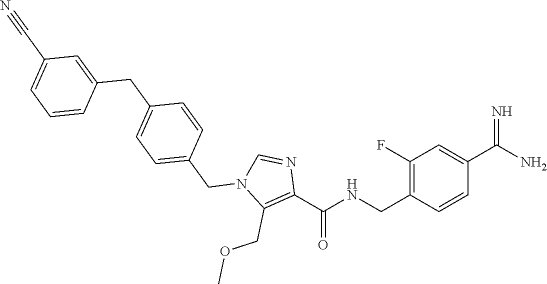 [SMILES: COCc1c(C(=O)NCc2ccc(C(=N)N)cc2F)ncn1Cc1ccc(Cc2cccc(C#N)c2)cc1] |
| 62 | 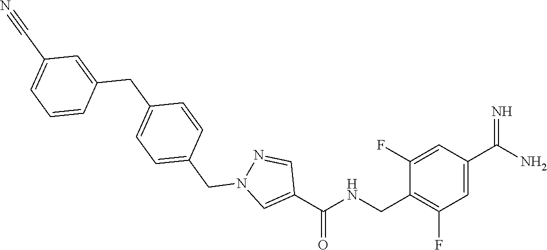 [SMILES: N#Cc1cccc(Cc2ccc(Cn3cc(C(=O)NCc4c(F)cc(C(=N)N)cc4F)cn3)cc2)c1] |
| 63 | 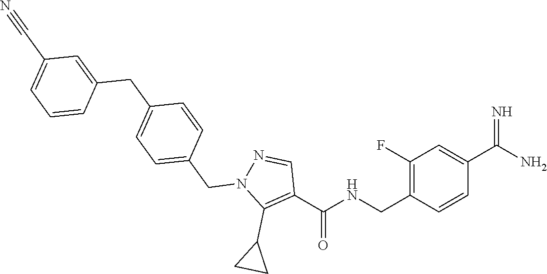 [SMILES: N#Cc1cccc(Cc2ccc(Cn3ncc(C(=O)NCc4ccc(C(=N)N)cc4F)c3C3CC3)cc2)c1] |
| 64 | 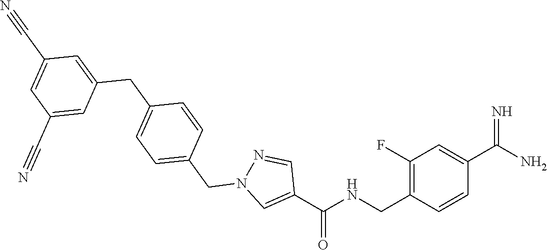 [SMILES: N#Cc1cc(C#N)cc(Cc2ccc(Cn3cc(C(=O)NCc4ccc(C(=N)N)cc4F)cn3)cc2)c1] |
| 65 | 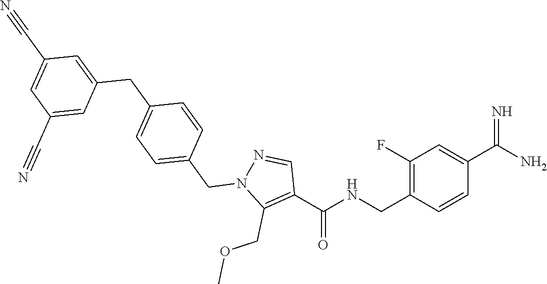 [SMILES: COCc1c(C(=O)NCc2ccc(C(=N)N)cc2F)cnn1Cc1ccc(Cc2cc(C#N)cc(C#N)c2)cc1] |
| 66 | 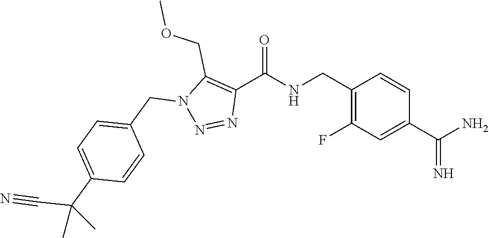 [SMILES: COCc1c(C(=O)NCc2ccc(C(=N)N)cc2F)nnn1Cc1ccc(C(C)(C)C#N)cc1] |
| 67 | 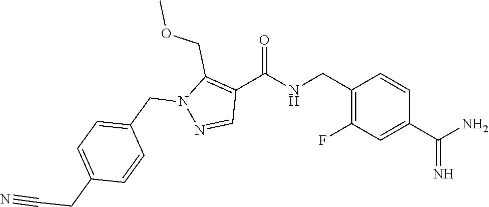 [SMILES: COCc1c(C(=O)NCc2ccc(C(=N)N)cc2F)cnn1Cc1ccc(CC#N)cc1] |
| 68 | 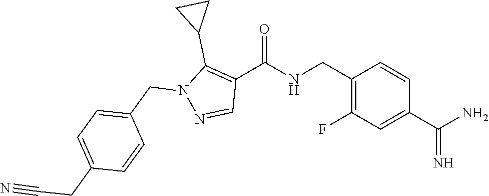 [SMILES: N#CCc1ccc(Cn2ncc(C(=O)NCc3ccc(C(=N)N)cc3F)c2C2CC2)cc1] |
| 69 | 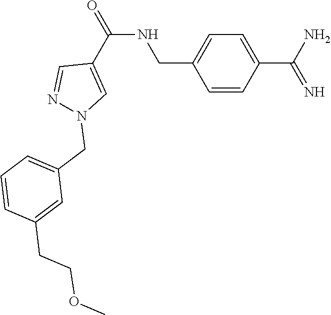 [SMILES: COCCc1cccc(Cn2cc(C(=O)NCc3ccc(C(=N)N)cc3)cn2)c1] |
| 70 | 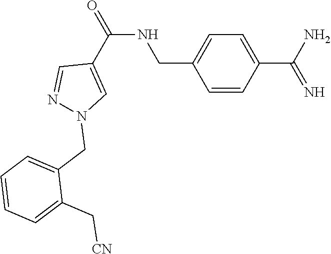 [SMILES: N#CCc1ccccc1Cn1cc(C(=O)NCc2ccc(C(=N)N)cc2)cn1] |
| 71 | 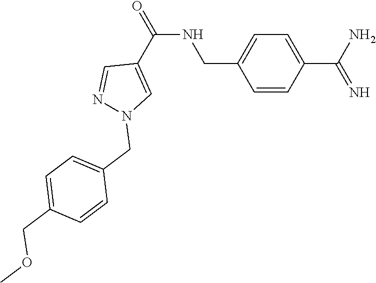 [SMILES: COCc1ccc(Cn2cc(C(=O)NCc3ccc(C(=N)N)cc3)cn2)cc1] |
| 72 | 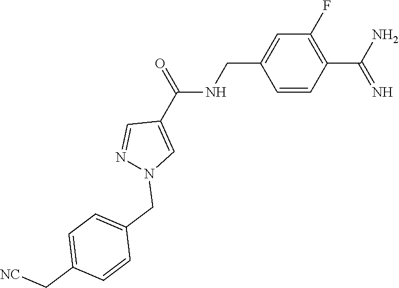 [SMILES: N#CCc1ccc(Cn2cc(C(=O)NCc3ccc(C(=N)N)c(F)c3)cn2)cc1] |
| 73 | 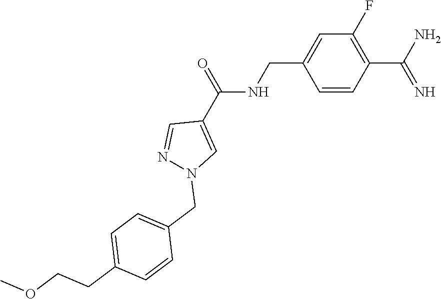 [SMILES: COCCc1ccc(Cn2cc(C(=O)NCc3ccc(C(=N)N)c(F)c3)cn2)cc1] |
| 74 | 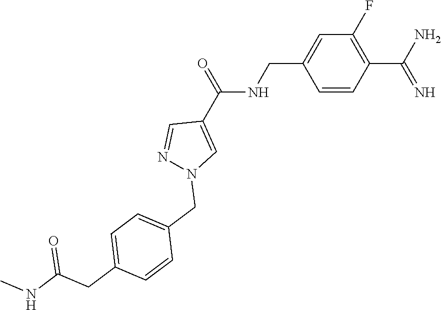 [SMILES: CNC(=O)Cc1ccc(Cn2cc(C(=O)NCc3ccc(C(=N)N)c(F)c3)cn2)cc1] |
| 75 | 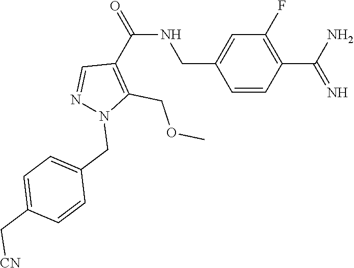 [SMILES: COCc1c(C(=O)NCc2ccc(C(=N)N)c(F)c2)cnn1Cc1ccc(CC#N)cc1] |
| 78 | 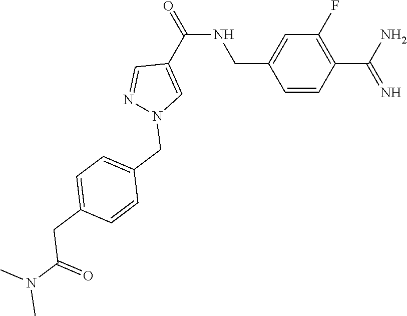 [SMILES: CN(C)C(=O)Cc1ccc(Cn2cc(C(=O)NCc3ccc(C(=N)N)c(F)c3)cn2)cc1] |
| 81 | 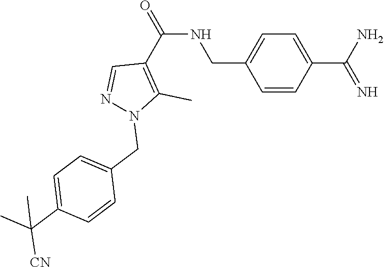 [SMILES: Cc1c(C(=O)NCc2ccc(C(=N)N)cc2)cnn1Cc1ccc(C(C)(C)C#N)cc1] |
| 82 | 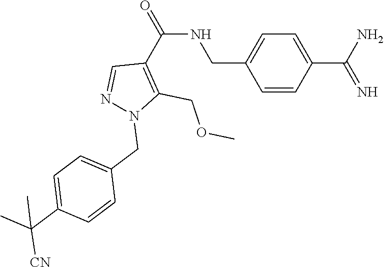 [SMILES: COCc1c(C(=O)NCc2ccc(C(=N)N)cc2)cnn1Cc1ccc(C(C)(C)C#N)cc1] |
| 83 | 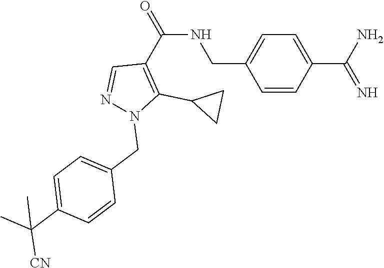 [SMILES: CC(C)(C#N)c1ccc(Cn2ncc(C(=O)NCc3ccc(C(=N)N)cc3)c2C2CC2)cc1] |
| 84 | 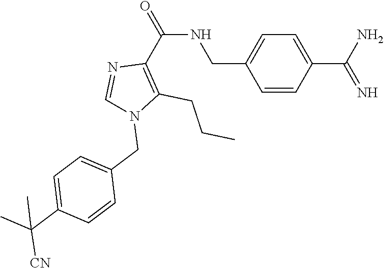 [SMILES: CCCc1c(C(=O)NCc2ccc(C(=N)N)cc2)ncn1Cc1ccc(C(C)(C)C#N)cc1] |
| 85 | 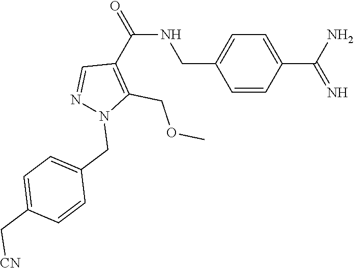 [SMILES: COCc1c(C(=O)NCc2ccc(C(=N)N)cc2)cnn1Cc1ccc(CC#N)cc1] |
| 86 | 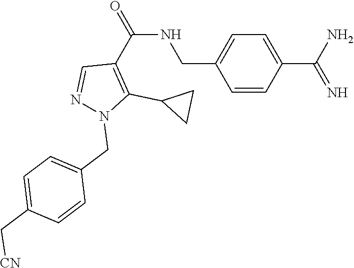 [SMILES: N#CCc1ccc(Cn2ncc(C(=O)NCc3ccc(C(=N)N)cc3)c2C2CC2)cc1] |
| 87 | 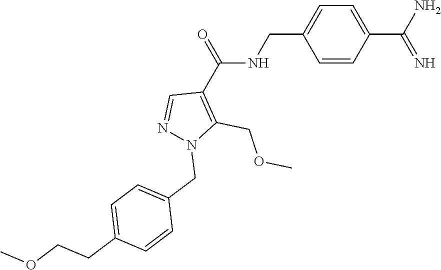 [SMILES: COCCc1ccc(Cn2ncc(C(=O)NCc3ccc(C(=N)N)cc3)c2COC)cc1] |

29. A pharmaceutical composition comprising a compound or pharmaceutically acceptable salt thereof according to claim 1 and a pharmaceutically acceptable carrier.

30. A method for treating a subject suffering from a disease or condition, comprising administering to the subject a compound or pharmaceutically acceptable salt thereof according to claim 1, wherein the disease or condition is selected from the group consisting of ischemic stroke, hemorrhagic stroke, hypertension, retinopathy, diabetic retinopathy, nephropathy, cerebral edema, pulmonary hypertension, inflammation, acute myocardial infarction, deep vein thrombosis, complications from fibrinolytic treatment, stroke, angina, angioedema, sepsis, arthritis, complications of cardiopulmonary bypass, capillary leak syndrome, inflammatory bowel disease, vascular complications from diabetes, diabetic macular edema, macular degeneration, neuropathy, age related macular degeneration, retinal vein occlusions, brain edema, ischemia reperfusion injury, angiogenesis, asthma, anaphylaxis, Alzheimer's disease, Parkinson's disease, multiple sclerosis, glioblastoma multiforme, complications of fibrinolysis treatment, increased albumin excretion, macroalbuminuria, pain, amyotrophic lateral sclerosis, Creutzfeld-Jakob disease, epilepsy, brain trauma, high altitude cerebral edema, cancer, disseminated intravascular coagulation, pancreatitis, inflammation, shock, hereditary angioedema (HAE), uveitis, polyangiitis, acute respiratory distress syndrome (ARDS), thrombosis, vasculitis, Crohn's disease, ulcerative colitis, enterocolitis, arteritis, glomerulonephritis, psoriasis, endometriosis, preeclampsia, malaria, arthritis, periodic and recurrent fever, Chagas disease, Reynaud's disease, systemic sclerosis, granulomatosis with polyangiitis, small vessel vasculitis, medium vessel vasculitis, large vessel vasculitis, pan-vasculitis, systemic autoinflammatory diseases, renal insufficiency, cerebral malaria, Clarkson's disease (systemic vascular leakage syndrome), Hantavirus infection, Hantavirus renal syndrome, Hantavirus pulmonary syndrome, viral associated inflammatory disorders, retinal vasculitis, uveitis, Eales' disease, Behcet's disease, sarcoidosis, whooping cough, coronavirus infection, and non-infectious posterior uveitis.

31. The method according to claim 30, wherein the disease or condition is selected from the group consisting of diabetic macular edema, diabetic retinopathy, and uveitis.
